(19) United States
(12) Patent Application Publication
(10) Pub. No.: US 2024/0127967 A1
(43) Pub. Date: Apr. 18, 2024

(54) METHODS OF MODELING IN VIVO EFFICACY OF DRUG COMBINATIONS FOR TREATMENT OF VIRAL INFECTIONS

(71) Applicants: University of Virginia Patent Foundation, Charlottesville, VA (US); Fred Hutchinson Cancer Research Center, Seatlle, WA (US); Stephen , J. , Polyak, Seattle, WA (US); Lisa , Johansen, Belmont, MA (US)

(72) Inventors: Judith M. White, Charlottesville, VA (US); Shuang Xu, Redmond, WA (US); Joshua T. Schiffer, Seattle, WA (US); Gene G. Olinger, Frederick, MD (US); Courtney L. Finch, Orrtanna, PA (US); Julie Dyall, Washington, DC (US); Stephen J. Polyak, Seattle, WA (US); Lisa Johansen, Belmont, MA (US)

(73) Assignees: Stephen J. Polyak, Seattle, WA (US); Lisa Johansen, Belmont, MA (US); University of Virginia Patent Foundation, Charlottesville, VA (US); Fred Hutchinson Cancer Research Center, Seatlle, WA (US)

(21) Appl. No.: 18/276,599

(22) PCT Filed: Feb. 9, 2022
(86) PCT No.: PCT/US2022/015835

§ 371 Date: Aug. 9, 2023

Related U.S. Application Data
(60) Provisional application No. 63/147,587, filed on Feb. 9, 2021.

(51) Int. Cl. G16H 70/40; A61K 31/138; A61K 31/40; A61K 45/06; A61P 31/14; G16H 50/50

(52) U.S. Cl. CPC G16H 70/40; A61K 31/138; A61K 31/40; A61K 45/06; A61P 31/14; G16H 50/50

(57) ABSTRACT

Methods of modeling the in vivo efficacy of drug combinations for the treatment or prevention of viral infection are described. The described methods combine data for single drugs and drug combinations from pharmacokinetic, pharmacodynamic, and viral dynamics models under a series of estimated in vivo drug potencies to provide predictions of the in vivo effects of the drug combinations. These predictions can be used to more accurately select drugs and drug treatment regimens that can be successful in controlling viral infection in animal studies, clinical trials and in medical or veterinary interventions. Also described is a method of treating or preventing filovirus infections using combinations of orally available drugs based on predictions from the modeling methods.

### CROSS-REFERENCE TO RELATED APPLICATIONS

This application claims benefit of U.S. Provisional Patent Application Ser. No. 63/147,587, filed Feb. 9, 2021, which is herein incorporated by reference in its entirety.

### STATEMENT OF GOVERNMENT INTEREST

This invention was made with government support under Grant No. AI114776 and Grant No. AI121129 awarded by the National Institutes of Health. The government has certain rights in the invention.

### TECHNICAL FIELD

The presently disclosed subject matter relates to methods of modeling in vivo antiviral potency of combinations of drugs, e.g., in order to provide enhanced prediction of drug combinations and drug combination dosing regimens that are effective in treating or controlling viral infections in vivo. The presently disclosed subject matter further relates to methods of treating viral infections, such as Ebolavirus, Marburgvirus, and other filovirus infections, using combinations of orally available drugs.

### BACKGROUND

Viruses continue to be important human and animal pathogens, causing a variety of diseases. The family Filoviridae contains human pathogens of high consequence including Ebola virus (EBOV), Sudan virus (SUDV) and Marburg virus (MARV) [1-3]. The largest outbreak of EBOV occurred in Western Africa between 2013 and 2016 leading to the deaths of over 11,000 individuals [2,4]. The second largest outbreak occurred in the northeastern region of the Democratic Republic of the Congo (DRC) between August 2018 and June 2020, and an eleventh outbreak recently occurred in the DRC (June to November 2020). There is now a licensed vaccine (sold under the tradename ERVEBO® (Merck Sharp & Dohme Corp., Whitehouse Station, New Jersey, United States of America)), a licensed therapeutic monoclonal antibody (mAb) (sold under the brandname Ebanga), and a licensed therapeutic mAb cocktail (sold under the trandename INMAZEB (Regeneron Pharmaceuticals, Tarrytown, New York, United States of America)) against EBOV. These undoubtedly played significant roles in curbing the latest outbreaks. However, no vaccines or therapeutics are currently approved to prevent or treat human diseases caused by other members of the Ebolavirus genus (e.g., SUDV; Bundibugyo virus, Tai Forest Virus), by members of the Marburgvirus genus, nor for diseases that might be caused by emerging filoviruses (e.g., Reference [5]).

Accordingly, there is an ongoing need for additional compositions and methods of treating viral infections, particularly compositions that can be administered orally and/or without the need for medical facilities or personnel. There is also an ongoing need for improved methods of predicting in vivo efficacy of drug combinations, e.g., to expedite or otherwise enhance drug development of antiviral treatments.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

In some embodiments, the presently disclosed subject matter provides a method of modeling an in vivo efficacy of a drug combination in the treatment of a viral infection of a target virus in a target host subject, the method comprising: (a) acquiring or preparing a first data set, wherein the first data set comprises pharmacokinetic (PK) data for a first drug and a second drug; (b) acquiring or preparing a second data set, wherein the second data set comprises pharmacodynamic (PD) data for the first drug, the second drug, and a combination of the first drug and the second drug; (c) acquiring or preparing a third data set, wherein the third data set comprises viral load dynamics data for the target virus in a relevant host subject; (d) combining the first, second, and third data sets using a plurality of projected in vivo drug potencies against the target virus for each of the first drug, the second drug, and the combination of the first drug and the second drug, thereby predicting a plurality of different viral load trajectories in the target host subject; and (e) analyzing the plurality of different viral load trajectories predicted in step (d) to predict in vivo efficacy of the combination of the first drug and the second drug at one or more of the plurality of projected in vivo drug potencies.

In some embodiments, the method further comprises administering the combination of the first drug and the second drug to the target subject or a surrogate animal subject thereof, wherein the administering comprises administering the combination of the first drug and the second drug at dose levels and/or dosing intervals predicted to be effective in vivo by the analyzing of step (e). In some embodiments, step (e) comprises predicting the maximum time interval between initial exposure of a target subject to the target virus and an initial dose administration of the combination of the first drug and the second drug to the subject that provides a predetermined level of control of in vivo viral load; and the method further comprises administering an initial dose of the combination of the first drug and the second drug to the target subject or a surrogate animal subject thereof within the predicted maximum time interval.

In some embodiments, the target virus is a virus selected from the group comprising arenaviruses, bunyaviruses, calciviruses, filoviruses, flaviviruses, orthomyxoviruses, picornaviruses, rhabdoviruses, togaviruses, influenza viruses and coronaviruses.

In some embodiments, step (b) comprises performing two-way concentration-dependent analysis of drug-drug interactions between the first drug and the second drug.

In some embodiments, the drug combination comprises a third drug, and the first data set comprises PK data for the third drug; the second data set comprises PD data for the third drug, a combination of the first drug and the third drug, a combination of the second drug and the third drug, and a combination of the first drug, the second drug and the third drug; step (d) comprises combining the first, second and third data sets using a plurality of projected in vivo drug potencies against the target virus for each of the first drug, the second drug, third drug, the combination of the first drug and the second drug, the combination of the first drug and the third drug, the combination of the second drug and the third drug, and the combination of the first drug, the second drug and the third drug; and step (e) comprises analyzing the plurality of different viral load trajectories predicted in step (d) to predict in vivo efficacy of the combination of the first drug and the second drug, the first drug and the third drug, the second drug and the third drug, and the first drug, the second drug and the third drug at one or more of the plurality of projected in vivo drug potencies.

In some embodiments, the presently disclosed subject matter provides a method of treating or preventing a filovirus infection in an animal subject in need thereof, the method comprising orally administering to said animal subject one of (a) a combination of bepridil or a pharmaceutically acceptable salt thereof and sertraline or a pharmaceutically acceptable salt thereof such that the combination provides a dose of 300 milligrams per day (mg/D) of bepridil and a dose of 200 mg/D of sertraline; or (b) a combination of sertraline or a pharmaceutically acceptable salt thereof and toremifene or a pharmaceutically acceptable salt thereof such that the combination provides a dose of 200 mg/D of sertraline and a dose of 150 mg/D of toremifene.

In some embodiments, the filovirus infection is an infection of an Ebolavirus species. In some embodiments, the Ebolavirus species is Zaire ebolavirus. In some embodiments, the filovirus infection is an infection of a Marburgvirus species. In some embodiments, the Marburgvirus species is Marburg marburgvirus.

In some embodiments, the animal subject is a human. In some embodiments, the orally administering is first performed within about 1 day of exposure or suspected exposure of the subject to a filovirus that causes the filovirus infection. In some embodiments, the administering is performed daily for ten days.

In some embodiments, the method further comprises administering one or more additional therapeutic agents to the subject. In some embodiments, the one or more additional therapeutic agents comprise an antiviral agent.

In some embodiments, the presently disclosed subject matter provides a composition for use in a method of treating or preventing a filovirus infection in an animal subject in need thereof, wherein the composition comprises: (a) a combination of bepridil or a pharmaceutically acceptable salt thereof and sertraline or a pharmaceutically acceptable salt thereof such that the combination provides a dose of 300 milligrams per day (mg/D) of bepridil and a dose of 200 mg/D of sertraline; or (b) a combination of sertraline or a pharmaceutically acceptable salt thereof and toremifene or a pharmaceutically acceptable salt thereof such that the combination provides a dose of 200 mg/D of sertraline and a dose of 150 mg/D of toremifene, and wherein the composition is administered orally to said animal subject. In some embodiments, the composition is provided in a dosage form wherein a combination preparation comprising a mixture of: (a) bepridil or a pharmaceutically acceptable salt thereof and sertraline or a pharmaceutically acceptable salt thereof, or (b) sertraline or a pharmaceutically acceptable salt thereof and toremifene or a pharmaceutically acceptable salt thereof, is provided in a single dosage form. In some embodiments, the composition is provided in a dosage form wherein two separate individual preparations of: (a) bepridil or a pharmaceutically acceptable salt thereof and sertraline or a pharmaceutically acceptable salt thereof, or (b) sertraline or a pharmaceutically acceptable salt thereof and toremifene or a pharmaceutically acceptable salt thereof, are provided in two separate single dosage forms.

Accordingly, it is an object of the presently disclosed subject matter to provide methods and/or compositions to treat or prevent viral infections and the diseases or conditions caused by the viral infections, and methods for modeling in vivo antiviral potency of combinations of drugs.

An object of the presently disclosed subject matter having been stated hereinabove, and which is achieved in whole or in part by the presently disclosed subject matter, other objects will become evident as the description proceeds hereinbelow.

### BRIEF DESCRIPTION OF THE DRAWINGS

FIGS. 1A-1D: Frameworks for studying the advantage of drug combinations against EBOV infection. (FIG. 1A) PK modeling (drug concentration in human plasma or serum over time). (FIG. 1B) PD modeling (drug concentration dependent inhibition of EBOV replication) of single drugs (top) and drug combinations (bottom). (FIG. 1C) EBOV dynamics model (plasma viral load over time in cynomolgus macaques, schematic styled after FIG. 3 in [29]). (FIG. 1D) The PK, PD (single drugs and drug combinations), and EBOV viral load dynamics model are combined under different assumptions of in vivo IC₅₀s (the plasma concentration of drug required to inhibit EBOV replication by 50%, the true in vivo drug potency against EBOV, in humans) to predict EBOV viral load trajectories in humans in the presence of single drugs or drug combinations.

FIGS. 2A-2C: Pharmacokinetic model of bepridil. (FIG. 2A) Schematic PK model of bepridil. Dose, D, of bepridil is orally administered into GI tract (A_{G}), and it is then absorbed into the plasma compartment (C_{P}, with volume V) following a zero-order absorption. Bepridil in plasma compartment (C_{P}) moves to the peripheral compartment (C₂) at rate k₁₂ and reversely at rate k₂₁. Bepridil is eliminated at rate k from the plasma compartment (C_{P}). (FIG. 2B) Differential equations describing the pharmacokinetics of bepridil in each compartment. (FIG. 2C) Projections of plasma bepridil levels and the average concentrations of bepridil measured in the plasma of volunteers following oral administration of 400 mg bepridil-HCl. (See Reference [35]).

FIGS. 3A-3C: Pharmacokinetic model of sertraline. (FIG. 3A) Schematic PK model of sertraline. Dose, D, of sertraline is orally administered into GI tract (A_{G}), and it is then absorbed into the Plasma compartment (C, with volume V) with delay, T_{lag}, at rate kₐ. Sertraline is eliminated from the Plasma compartment (C) following Michaelis Menten reaction with Vₘ and Kₘ. (FIG. 3B) Differential equations describing the PK of sertraline in each compartment. (FIG. 3C) Projections of plasma sertraline levels and the average concentrations of sertraline measured in the plasma of volunteers following oral administration of 100 mg of sertraline-HCl. (See Reference [37]).

FIGS. 4A-4C: Pharmacokinetic model of toremifene. (FIG. 4A) Schematic PK model of toremifene. Dose, D, of toremifene is orally administered into GI tract (A_{G}), and it is then absorbed into the Serum compartment (Cₛ, with volume V) at rate kₐ. Toremifene in serum compartment (Cₛ) moves to the Peripheral compartment (C₂) at rate k₁₂ and reversely at rate k₂₁. Toremifene is eliminated at rate k from the Serum compartment (Cₛ). (FIG. 4B) Differential equations describing the PK of toremifene in each compartment. (FIG. 4C) Projections of serum toremifene levels and the average concentrations of toremifene measured in the serum of volunteers following oral administration of 50 or 100 mg of toremifene. (See Reference [36]).

FIGS. 5A-5C: Pharmacodynamic projections of the efficacies of single drugs ((FIG. 5A) bepridil; (FIG. 5B) sertraline; and (FIG. 5C) toremifene) assuming various in vivo IC₅₀s. Lines indicate the assumptions of an in vivo IC₅₀s as follows (from left to right): 0.1×, 1×, 5×, and 10× of in vitro IC₅₀. Antiviral efficacy of a single drug changes according to its drug concentration.

FIG. 6: Apilimod does not protect mice challenged with mouse adapted EBOV (ma-EBOV). Female C56BL/6 mice (10/group) were treated with the indicated dose of apilimod (in 0.9% saline) or vehicle control. Approximately 4 hours later they were infected with 246 PFU of ma-EBOV. They were then treated daily with the same dose of apilimod, prepared freshly from a pre-weighed and stored packet (or vehicle), for an additional 9 days. The graph shows daily survival rates. A PK study in mice revealed that a single dose of 10 mg/kg apilimod administered IP resulted in a Cₘₐₓ of 2.53 μM, which is well above the in vitro IC₅₀ against EBOV in Huh7 cells (see Table 9).

FIGS. 7A and 7B: Activity of bepridil, sertraline and toremifene in Vehicle 8 (V8) against VSV-EBOV-GPΔ-Luc over time. Bepridil-HCl (50 mg/mL), sertraline-HCl (6 mg/mL), and toremifene citrate (8.5 mg/mL) were prepared in V8 on day 1 (d1) as described in the Examples, and stored at 4° C. for (FIG. 7A) 15 or (FIG. 7B) 8 days. On the indicated day the stock was brought to RT. HEK293T/17 cells were pretreated with drugs as indicated, infected with VSV-EBOV-GPΔ-Luc, and scored for infection as described in the Examples. All samples were analyzed in triplicate, and the data are averages +/−sd.

FIGS. 8A-8C: Activity of bepridil (FIG. 8A), sertraline (FIG. 8B) and toremifene (FIG. 8C) in V8 against EBOV/Mak over seven days. Drug stocks were prepared on d0 in V8 as for FIGS. 7A and 7B, stored at 4° C. (left bar of each pair of bars) or RT (right bar of each pair of bars), and then tested in 8 point dose response curves on d0, d3, d5, and d7 using 2-fold serial dilutions in V8. Huh7 cells were pretreated with the indicated drugs and infected with EBOV/Mak (moi 0.21). Two experiments were conducted; for each, dose response curves were run on duplicate plates with 3 replicates of each dose on each plate and analyzed to obtain IC₅₀s as described in the Examples. Data from the triplicate technical replicates on each plate were averaged. The data shown are the averages of the average IC₅₀s +/−sd from two to four plates. Data from plates with Z′ factor <0.2 were not included.

FIGS. 9A-9C: Activity of bepridil (FIG. 9A), sertraline (FIG. 9B) and toremifene (FIG. 9C) in DMSO (D) and vehicle 8 (V8) against EBOV/Mak and ma-EBOV. Drug treatments and infections were conducted as described in the Examples and legend for FIGS. 8A-8C. The data shown are the average IC₅₀s+/−sd from four plates excepting one plate with Z′ factor <0.2.

FIG. 10: Bepridil, sertraline and toremifene (in V8) are well-tolerated by mice. Female C57BL/6 mice (5/group) were administered the indicated doses of bepridil, sertraline and toremifene in V8 by oral gavage for 11 consecutive days. Daily average weights for 18 days for each group are shown +/−sd.

FIGS. 11A-11D: Empirical and projected efficacies of drug combinations. (FIGS. 11A and 11C) Empirically quantified efficacies of (bepridil HCl+sertraline HCl) and (sertraline HCl+toremifene citrate) in Huh7 cells taken from reference [22]. Highly synergistic regions are highlighted in the boxes bounded by stars, which have ΔBliss values <−0.3. (FIGS. 11B and 11D) Model-projected efficacies of (bepridil HCl+sertraline HCl) and (sertraline HCl+toremifene citrate) using the Bliss model. Numbers in the boxes of the heatmap represent the percentage of inhibition, with 0 for no inhibition and 100 for complete inhibition of EBOV replication.

FIGS. 12A-12I. Projections of pharmacokinetics (PK), pharmacodynamics (PD), and EBOV viral load dynamics when sertraline (S) and bepridil (B) are used alone and together. Treatment periods start on day 0 and end on day 10, as indicated with gray backgrounds. Doses are 200 mg/day for the single sertraline treatment (FIGS. 12A, 12D, and 12G), 300 mg/day for the single bepridil treatment (FIGS. 12B, 12E and 12H), and 200 mg/day sertraline-300 mg/day bepridil for the treatment of the drug combination (FIGS. 12C, 12F, and 12I). Various in vivo IC₅₀s were assumed in each treatment: 10×, 5×, 1× and 0.1× of in vitro IC₅₀s (FIGS. 12D-12I). (FIGS. 12A-12C) PK projections of sertraline (S) and bepridil (B) and their combination. Note: The concentration of sertraline equals 0 μM over time in the single bepridil therapy (FIG. 12B), while it is greater than 0 μM (˜0.25 μM) during the treatment period (gray shaded region) in the drug combination therapy (FIG. 12C). (FIGS. 12D-12F) Projected efficacies of single sertraline, single bepridil, and their combination under assumptions of various in vivo IC₅₀s. (FIGS. 12G-12I) Model-simulated EBOV viral load dynamics (log₁₀(Cp/mL), log₁₀ (copy number/mL)) under different treatment regimens: single sertraline, single bepridil and their combination, when in vivo IC₅₀s were varied. In graphs in FIGS. 12G-12I, in cases where curves overlap (e.g., for the 5× and 10× simulations of in vivo IC₅₀s), the lines were shifted just enough to better visualize the predicted viral loads. Note: a straight line at zero (e.g., FIGS. 12H and 12I) indicates a lack of detectable remaining virus.

FIGS. 13A-13I: Projections of pharmacokinetics (PK), pharmacodynamics (PD), and EBOV viral dynamics when sertraline (S) and toremifene (T) are used alone and together. Treatment periods start on day 0 and end on day 10, as indicated with gray backgrounds. Doses are 200 mg/day for the single sertraline treatment (FIGS. 13A, 13D, and 13G), 150 mg/day with a loading dose of 300 mg/day (on day 0) for the single toremifene treatment (FIGS. 13B, 13E, and 13H), and 200 mg/day sertraline-150 mg/day toremifene with a loading dose of 300 mg/day (on day 0) for the treatment of the drug combination (FIGS. 13C, 13F, and 13I). Various in vivo IC₅₀s were assumed in each treatment: 10×, 5×, 1× and 0.1× of in vitro IC₅₀s (FIGS. 13D-13I). (FIGS. 13A-13C) PK prof ections of sertraline (S) and toremifene (T). (FIGS. 13D-13F) Projected efficacies of single sertraline, single toremifene, and their combination under assumptions of various in vivo IC₅₀s. (FIGS. 13G-13I) Model-simulated EBOV viral load dynamics (log₁₀(Cp/mL), log₁₀(copy number/mL)) in different treatments: single sertraline, single toremifene and their combination, when in vivo IC₅₀s were varied. In FIGS. 13G-13I, in cases where curves overlap (e.g., for the 5× and 10× simulations of in vivo IC₅₀s), the lines were shifted just enough to better visualize the predicted viral loads. Note: a straight line at zero (e.g., in FIG. 13I) indicates a lack of detectable remaining virus.

FIGS. 14A-14D: Two-way pharmacodynamic (PD) modeling approach for the efficacy of drug combinations comprising a first drug, drug 1, and a second drug, drug 2. (FIG. 14A) In Model A, drug 1 is the centric drug, and drug 2 is the secondary drug. E_{combo} equals the sum of the baseline efficacy (E_{2|1=0}) provided by drug 2 alone and the efficacy of drug 1 (E_{1|2≥0}) with drug 2 dependent PD parameters. (FIG. 14B) Dose response curves of the combined efficacy at different concentrations of drug 2 (C₂) when drug 2 is considered the secondary drug. Threshold is defined as the concentration of the secondary drug when it provides a baseline efficacy of 1 (100%). (FIG. 14C) In Model B, drug 1 is the secondary drug, and drug 2 is the centric drug. E_{combo} equals the sum of the baseline efficacy (E_{1|2=0}) provided by drug 1 alone and the efficacy of drug 2 (E_{2|1≥0}) with drug 1 dependent PD parameters. (FIG. 14D) Dose response curves of the combined efficacy at different concentrations of drug 1 (C₁), when drug 1 is considered the secondary drug. Performance of model A and B can be compared based on SSE (sum of squared error) and R², with smaller SSE and higher R² indicating better model prediction compared with empirical dataset. PD parameters of the centric drug in Model A and B can be modeled as non-mechanistic functions (fᵢ) of the concentration of the secondary drug (C₁ or C₂).

FIGS. 15A-15J: Two-way pharmacodynamic modeling for the efficacy of (sertraline+bepridil) combination against Ebola virus. (FIG. 15A) Empirically quantified E_{combo} of (sertraline+bepridil) combination in Huh7 cells taken from reference [22]. In Model A, sertraline is the centric drug, and bepridil is the secondary drug. (FIG. 15B) Efficacy of bepridil assuming sertraline is absent (E_{B|S=0}). (FIG. 15C) Efficacy of sertraline with bepridil dependent pharmacodynamic parameters (E_{S|B>0}). (FIG. 15D) Predicted E_{combo} by model A (E_{combo,A}=E_{S|B≥0}+E_{B|S=0}). (FIG. 15E) Predicted change in E_{combo,A} (ΔE_{A}=E_{combo,A}−min (100, (E_{S|B=0}+E_{B|S=0})). In Model B, bepridil is the centric drug, and sertraline is the secondary drug. (FIG. 15F) Efficacy of sertraline assuming bepridil is absent (E_{B|S=0}). (FIG. 15G) Efficacy of bepridil with sertraline dependent pharmacodynamic parameters (E_{B|S≥0}). (FIG. 15H) Predicted E_{combo} by model B (E_{combo,B}=E_{B|S≥0}+E_{S|B=0}). (FIG. 15I) Predicted change in E_{combo,B} (ΔE_{B}=E_{combo,B}−min (100, (E_{S|B=0}+E_{B|S=0})). (FIG. 15J) Model comparison among Model A, Model B and the PD model described in Example 5 (i.e., (E_{combo}=100−(100×fᵤ₁×fᵤ₂)^{a}). Highly synergistic regions, with ΔBliss values <−0.3, are highlighted in boxes. Percentage of inhibition ranges from 0 for no inhibition to 100 for complete inhibition of EBOV replication. Changes in E_{combo} (ΔE) compared with single drugs are in the range of −70 to 70.

FIGS. 16A-16J: Two-way pharmacodynamic modeling for the efficacy of (sertraline+toremifene) combination against Ebola virus. (FIG. 16A) Empirically quantified E_{combo} of (sertraline+toremifene) combination in Huh7 cells taken from reference [22]. In Model A, sertraline is the centric drug, and toremifene is the secondary drug. (FIG. 16B) Efficacy of toremifene when sertraline is absent (E_{T|S=0}). (FIG. 16C) Efficacy of sertraline with toremifene dependent pharmacodynamic parameters (E_{S|T≥0}). (FIG. 16D) Predicted E_{combo} by model A (E_{combo,A}=E_{S|T≥0}+E_{T|S=0}). (FIG. 16E) Predicted change in E_{combo,A} (ΔE_{A}=E_{combo,A}−min (100, (E_{S|T=0}+E_{T|S=0})). In Model B, toremifene is the centric drug, and sertraline is the secondary drug. (FIG. 16F) Efficacy of sertraline when toremifene is absent (E_{S|T=0}). (FIG. 16G) Efficacy of toremifene with sertraline dependent pharmacodynamic parameters (E_{T|S≥0}). (FIG. 16H) Predicted E_{combo} by model B (E_{combo,B}=E_{T|S≥0}+E_{S|T=0}). (FIG. 16I) Predicted change in E_{combo,B} (ΔE_{B}=E_{combo,B}−min (100, (E_{S|T=0}+E_{T|S=0})). (FIG. 16J) Model comparison among Model A, Model B and the PD model described in Example 5 (i.e., (E_{combo}=100−(100×fᵤ₁×fᵤ₂)^{a})). Highly synergistic regions, with ΔBliss values <−0.3, are highlighted in boxes. Percentage of inhibition ranges from 0 for no inhibition to 100 for complete inhibition of EBOV replication. Changes in E_{combo} (ΔE) compared with single drugs are in the range of −70 to 70.

FIGS. 17A-17J: Two-way pharmacodynamic modeling for the efficacy of (nitazoxanide+remdesivir) combination against severe acute respiratory syndrome coronavirus 2 (SARS-CoV-2). (FIG. 17A) Empirically quantified E_{combo} of (nitazoxanide +remdesivir) combination in Vero E6 cells taken from reference [86]. In Model A, nitazoxanide is the centric drug, and remdesivir is the secondary drug. (FIG. 17B) Efficacy of remdesivir when nitazoxanide is absent (E_{R|N=0}). (FIG. 17C) Efficacy of nitazoxanide with remdesivir dependent pharmacodynamic parameters (E_{N|R≥0}). (FIG. 17D) Predicted E_{combo} by model A (E_{combo,A}=E_{N|R≥0}+E_{R|N=0}). (FIG. 17E) Predicted change in E_{combo,A} (ΔE_{A}=E_{combo,A}−min (100, (E_{N|R=0}+E_{R|N=0})). In Model B, remdesivir is the centric drug, and nitazoxanide is the secondary drug. (FIG. 17F) Efficacy of nitazoxanide when remdesivir is absent (E_{N|R=0}). (FIG. 17G) Efficacy of remdesivir with nitazoxanide dependent pharmacodynamic parameters (E_{R|N≥0}). (FIG. 17H) Predicted E_{combo} by model B (E_{combo,B}=E_{R|N≥0}+E_{N|R=0}). (FIG. 17I) Predicted change in E_{combo,B} (ΔE_{B}=E_{combo,B}−min (100, (E_{N|R=0}+E_{R|N=0})). (FIG. 17J) Model comparison among Model A, Model B and the PD model of Example 5 (i.e., (E_{combo}=100−(100×fᵤ₁×fᵤ₂)^{a}). Highly synergistic regions, with HSA scores <−0.3, are highlighted in boxes. Percentage of inhibition ranges from 0 for no inhibition to 100 for complete inhibition of EBOV replication. Changes in E_{combo} (ΔE) compared with single drugs are in the range of −70 to 70.

FIGS. 18A-18J: Two-way pharmacodynamic modeling for the efficacy of (nitazoxanide+arbidol) combination against SARS-CoV-2. (FIG. 18A) Empirically quantified E_{combo} of (nitazoxanide+arbidol) combination in Vero E6 cells taken from reference [86]. In Model A, nitazoxanide is the centric drug, and arbidol is the secondary drug. (FIG. 18B) Efficacy of arbidol when nitazoxanide is absent (E_{A|N=0}). (FIG. 18C) Efficacy of nitazoxanide with arbidol dependent pharmacodynamic parameters (E_{N|A≥0}). (FIG. 18D) Predicted E_{combo} by model A (E_{combo,A}=E_{N|A≥0}+E_{A|N=0}). (FIG. 18E) Predicted change in E_{combo,A} (ΔE_{A}=E_{combo,A}−min (100, (E_{N|A=0}+E_{A|N=0})). In Model B, arbidol is the centric drug, and nitazoxanide is the secondary drug. (FIG. 18F) Efficacy of nitazoxanide when arbidol is absent (E_{N|A=0}). (FIG. 18G) Efficacy of arbidol with nitazoxanide dependent pharmacodynamic parameters (E_{A|N≥0}). (FIG. 18H) Predicted E_{combo} by model B (E_{combo,B}=E_{A|N≥0}+E_{N|A=0}) (FIG. 18I) Predicted change in E_{combo,B} (ΔE_{B}=E_{combo,B}−Min (100, (E_{N|A=0}+E_{A|N=0})). (FIG. 18J) Model comparison among Model A, Model B and the PD model described in Example 5 (i.e., (E_{combo}=100−(100×fᵤ₁×fᵤ₂)^{a}). Highly synergistic regions, with HSA scores <−0.3, are highlighted in boxes. Antagonistic regions, with HAS≥0.06, are also highlighted in boxes. Percentage of inhibition ranges from 0 for no inhibition to 100 for complete inhibition of EBOV replication. Changes in E_{combo} (ΔE) compared with single drugs are in the range of −70 to 70.

FIG. 19A-19L: Secondary drug affects the centric drug's pharmacodynamic parameters in anti-Ebola and anti-SARS-CoV-2 drug combinations. (FIGS. 19A-19C) Estimated Eₘₐₓ, IC₅₀ and hill coefficient of the centric drug in two way pharmacodynamic modeling for (sertraline+bepridil), with sertraline as the centric drug in model A and bepridil as the centric drug in model B. (FIGS. 19D-19F) Estimated Eₘₐₓ, IC₅₀ and hill coefficient of the centric drug in two way pharmacodynamic modeling for (sertraline+toremifene), with sertraline as the centric drug in model A and toremifene as the centric drug in model B. (FIGS. 19G-19I) Estimated Eₘₐₓ, IC₅₀ and hill coefficient of the centric drug in two way pharmacodynamic modeling for (nitazoxanide+remdesivir), with remdesivir as the centric drug in model A and nitazoxanide as the centric drug in model B. (FIGS. 19J-19L) Estimated Eₘₐₓ, IC₅₀ and hill coefficient of the centric drug in two way pharmacodynamic modeling for (nitazoxanide+arbidol), with arbidol as the centric drug in model A and nitazoxanide as the centric drug in model B.

FIG. 20: Biphasic effects of sertraline on the efficacy of 2.5 μM bepridil.

FIG. 21. Schematic flow diagram of an exemplary method of modeling the in vivo antiviral efficacy of a combination of drugs according to the presently disclosed subject matter.

### DETAILED DESCRIPTION

The presently disclosed subject matter will now be described more fully. The presently disclosed subject matter can, however, be embodied in different forms and should not be construed as limited to the embodiments set forth herein below and in the accompanying Examples. Rather, these embodiments are provided so that this disclosure will be thorough and complete, and will fully convey the scope of the embodiments to those skilled in the art.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

### I. Definitions

While the following terms are believed to be well understood by one of ordinary skill in the art, the following definitions are set forth to facilitate explanation of the presently disclosed subject matter.

Unless defined otherwise, all technical and scientific terms used herein have the same meaning as commonly understood to one of ordinary skill in the art to which the presently disclosed subject matter belongs.

Following long-standing patent law convention, the terms “a”, “an”, and “the” refer to “one or more” when used in this application, including the claims.

The term “and/or” when used in describing two or more items or conditions, refers to situations where all named items or conditions are present or applicable, or to situations wherein only one (or less than all) of the items or conditions is present or applicable.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” can mean at least a second or more.

The term “comprising”, which is synonymous with “including,” “containing,” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

Unless otherwise indicated, all numbers expressing quantities of time, concentration, dosage and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about”, when referring to a value is meant to encompass variations of in one example ±20% or ±10%, in another example ±5%, in another example ±1%, and in still another example ±0.1% from the specified amount, as such variations are appropriate to perform the disclosed methods.

As use herein, the terms “administration of” and/or “administering” a compound or composition can be understood to refer to providing a compound or composition (e.g., targeted liposomes comprising an active agent, such as a drug) of the presently disclosed subject matter to a subject in need of treatment. As used herein “administering” includes administration of a compound or composition by any number of routes and modes including, but not limited to, topical, oral, buccal, intravenous, intramuscular, intra-arterial, intramedullary, intrathecal, intraventricular, transdermal, subcutaneous, intraperitoneal, intranasal, enteral, topical, sublingual, vaginal, ophthalmic, pulmonary, vaginal, and rectal approaches.

As used herein, an “effective amount” or “therapeutically effective amount” refers to an amount of a compound or composition sufficient to produce a selected effect, such as but not limited to alleviating symptoms of a condition, disease, or disorder. In the context of administering a compound or composition in the form of a combination, such as multiple compositions, the amount of each a compound or composition, when administered in combination with one or more other compositions, may be different from when that composition is administered alone. Thus, an effective amount of a combination of compounds or compositions refers collectively to the combination as a whole, although the actual amounts of each a compound or composition may vary. The term “more effective” means that the selected effect occurs to a greater extent by one treatment relative to the second treatment to which it is being compared.

The term “prevent”, as used herein, means to stop something from happening, or taking advance measures against something possible or probable from happening. In the context of medicine, “prevention” generally refers to action taken to decrease the chance of getting a disease or condition. It is noted that “prevention” need not be absolute, and thus can occur as a matter of degree.

The terms “treatment” and “treating” as used herein refer to both therapeutic treatment and prophylactic or preventative measures, wherein the object is to prevent or slow down (lessen) the targeted pathologic condition, prevent the pathologic condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented.

The methods and compositions disclosed herein can be used on or involve predicted use in a subject (or host subject, i.e. living organism, such as a patient being treated or to be treated for a viral infection). In some embodiments, the subject is a human subject, although it is to be understood that the principles of the presently disclosed subject matter indicate that the presently disclosed subject matter is effective with respect to all vertebrate species, including mammals, which are intended to be included in the terms “subject” and “patient”. Moreover, a mammal is understood to include any mammalian species for which employing the compositions and methods disclosed herein is desirable, particularly agricultural and domestic mammalian species.

As used herein, an “analog” of a chemical compound is a compound that, by way of example, resembles another in structure but is not necessarily an isomer (e.g., 5-fluorouracil is an analog of thymine).

### II. General Considerations

Drug combinations are used for several viral diseases in humans, including human immunodeficiency virus (HIV) and hepatitis C virus (HCV) [87, 88]. In addition to diminished risk of drug resistance, drug synergy can provide therapeutic antiviral effects while minimizing side effects caused by higher required drug concentrations of monotherapies [22, 89-91]. In particular, drug combinations can have a role in the treatment of (re)emerging viral diseases with pandemic potential that currently lack effective prophylactic or therapeutic measures [28, 92-95]. Ideal treatments can include combinations of drugs that could be deployed widely and early during an outbreak as prophylaxis and early treatment. In conjunction with other non-pharmaceutical interventions, in-home use drug cocktails could limit the burden on health care systems and thwart person-to-person virus spread by lowering viral loads and the virus' ability to adapt to the host.

Accordingly, of particular interest in preparedness for future pandemics or virus outbreaks is the development of highly effective and widely available antiviral treatments for non-hospitalized patients. Thus, development of treatments involving oral and/or inhaled drugs, which could be taken at home as immediate post-exposure prophylaxis or early during illness to rapidly lower viral loads and subsequent harmful immune-activation is desirable. Additionally, drug combinations that can potentially reduce emergence of drug-resistant mutants and, through multiplicative or synergistic effects, bring drug doses into therapeutic range and mitigate side effects associated with high doses of single drugs, are a priority. These drug combinations can include directly acting antivirals (DAAs) as well as host-targeting agents (HTAs). Also of interest is the development of drugs that are approved or in advanced clinical testing, to allow a more rapid regulatory review process. For longer term planning purposes, drugs currently in pre-clinical development can also be considered. Suitable development plans can also prioritize drugs whose effective concentrations in relevant human tissue models are considerably below their toxic concentrations and in the range of attainable levels throughout the dosing interval. In addition, development can employ mathematical modeling at critical junctures of advancing to animal testing and clinical trial design. The presently disclosed subject matter provides methods and approaches that address these aspects set forth immediately above in this paragraph.

More generally, to prioritize combinations of drugs for use in treating viral infection, there are several aspects to consider. A first step can be to identify drug pairs with enhanced combinatorial potency. Typically, a drug combination study is performed involve a checkerboard assay in which varying doses of two drugs are set in a matrix and these defined dose mixtures (thirty-six for a 6×6 matrix) are tested for inhibition of viral infection in cells. These assays assess whether drug interactions are antagonistic, neutral, additive, multiplicative or synergistic, in increasing order of desirability. While synergy is always in theory preferable, its highest potential is in the context of single agents that have insufficient potency on their own. Antagonistic pairs should generally be excluded from further consideration. While many DAAs are expected to function independently of cell type, testing in more relevant cell systems (e.g., lung cells for testing respiratory viruses) can be of use for drugs that target entry. The presently disclosed subject matter provides methods and approaches that address these aspects set forth immediately above in this paragraph.

Synergy occurs when observed potency exceeds that of the expected combination effect based on a selected synergy model. In one representative sense, synergy denotes observed potency exceeding that predicted by a Bliss independence mathematical model, which assumes multiplicative effects of paired drugs [20].

An additional consideration is the molecular target(s) of the drugs in the combination and how the drugs impinge on virus lifecycle. For example, compounds that target the same viral protein tend to only have additive effects in which the second agent adds little to overall potency. The human exposure potential of the drugs is a further consideration. To leverage pairwise synergy for potential therapeutic use, the pharmacokinetic (PK) profiles of component drugs can be assessed. For instance, peak and trough drug concentrations, which are determined by tissue clearance kinetics, can be an important aspect. Ideally, both drugs would achieve levels that allow Bliss independence or synergy throughout the dosing interval. If there are only brief time windows of drug synergy, then synergy can have limited beneficial effects for infections, such as SARS-CoV-2, that are defined by rapid replication and spread dynamics [124].

Of note, in situations where a given concentration of a single agent already eliminates nearly 100% of new cell infections, synergy can have limited additional benefit. This possibility highlights the need to factor in projected peak and trough concentrations of both relevant drugs relative to their EC₅₀s to assess whether multiplicative or synergistic pharmacodynamics (PD) provides meaningful additional benefit. As an additional note of caution, it has been demonstrated that in vitro EC₅₀ values often overestimate antiviral potency in vivo such that in order to suppress virus in people, 5-10 times higher drug levels are required than predicted by cell culture assays [40]. It can also be useful to consider each drug's binding to protein in vitro and in vivo, as this can influence the amount of drug available [125,126].

Modeling approaches can be helpful by providing additional context for the potential benefits of combination effects that are additive, multiplicative, or synergistic. By accounting for non-linear drug levels over time, modeling can capture the proportion of time during which levels of both drugs provide potent inhibition of cellular infection, either by virtue of single drug potency or synergy. This is useful because it can identify scenarios in which addition of a second drug is helpful to achieve adequate viral suppression and those in which a second agent is unnecessary and could confer unnecessary toxicity.

Moreover, modeling can capture possible differential impacts of therapies in various hosts. For instance, whereas most infected people with SARS-CoV-2 infections, even those with critical illness, appear to eliminate high grade viral replication within 1-2 weeks [127], immunocompromised hosts can shed infectious SARS-CoV-2 at high viral loads for months [128-133], taking on a phenotype more consistent with chronic, persistent viruses like HIV. Here the virus undergoes considerable mutation and is more likely to become resistant to small molecule or mAb therapy. Modeling is well poised to capture the added potency required from synergistic agents in this specific clinical context.

Returning more particularly to the development of drug combinations for pandemic preparedness, members of eleven virus families have been designated as being of potential high consequence by the National Institute of Allergy and Infectious Disease (MAID), and it is of interest to develop oral (and inhaled, for respiratory viruses), thermally stable, inexpensive, preferably pan-family, drug cocktails to combat them [134]. Families of concern contain single-stranded RNA and are inhibited by one or more of the following polymerase inhibitors: RDV, MPV, favipiravir, or galidesivir (BCX4430). The members of nine of these families, including coronaviruses, are enveloped and hence deliver their genomes into the cytoplasm to initiate replication by a stereotypical membrane fusion process mediated by a fusion glycoprotein [57,135]. Many of these (e.g., the HAs of influenza viruses and the GPs of arenaviruses) also bind particles to the host cell surface, whereas other viruses contain a separate attachment/receptor binding protein. Both virus attachment and virus fusion are good targets for small molecule intervention [136-141]. As polymerase complexes are clearly excellent therapeutic targets [19,21,77], a ‘starter’ pan-family cocktail could include a drug(s) targeting viral glycoprotein(s) and a drug(s) targeting viral polymerase(s). Flaviviruses and togaviruses also encode proteases that, like the SARS-CoV-2 proteases, process their polyproteins during virus maturation [142,143]. Proteases therefore represent additional targets for a subset of category A-C viral pathogens.

The members of nine of the eleven virus families (i.e., arena, bunya-, calci-, filo, flavi, orthomyxo-, picorna-, rhabdo- and togaviruses) enter cells through endosomes [57], and hence the endosomal pathway is a target for their therapeutic intervention [144,145]. Indeed, many drug screens have uncovered endosome-targeting drugs against these pathogens. Endosomal features that can be targeted are virus particle internalization (e.g., via clathrin or macropinocytosis) as well as endosome trafficking, maturation, and composition including low pH, cathepsin proteases, Ca++ and other ions, and specific lipids [32,146,147]. Even Nipah and Hendra, which are category C paramyxoviruses that fuse at the plasma membrane, require a low pH-activated endosomal cathepsin to process their fusion glycoproteins and form infectious particles [148]. Furthermore, endosome-targeted drugs could inhibit infections by CoVs in tissues outside of the lung [149-151].

While DAAs are considered generally considered ideal based on their generally higher potency and selective indices, targeting host proteins critically involved in the viral lifecycle, including drugs that target endosomes for most high consequence viruses, is a consideration in combination therapies, especially with regard to preparing for the short-term for endosome-entering viruses. As described herein, including in the Examples, based on PK and other considerations two pairs of drugs (bepridil+sertraline and sertraline+toremifene) were vetted for eventual testing in an oral formulation in mice against a lethal Ebola virus challenge. The components of these pairs are approved, endosome-affecting drugs; each had previously been shown to protect mice (50-100%) in a lethal challenge model when given intraperitoneally [6,7]. As further described herein, including in the Examples, mathematical modeling of PK and PD showed that, compared to their individual constituents, both synergistic drug pairs have superior potential to reduce viral loads in humans. All three compounds in these drug pairs have also been shown to bind to a pocket in the Ebola virus GP thereby affecting its stability [55]. Hence some endosome-targeting drugs may also act directly on viral glycoproteins [72,98,152,153] i.e., be both HTAs and DAAs. Other host factors to consider for targeting include host proteases that prime viral glycoproteins for fusion [57,135,154,155], other host proteins involved in virus entry [156], host cell kinases [157-160], and host proteins involved in viral RNA production [78,161], nuclear export [162,163], and virus assembly and egress [164,165]. Given common strategies for many viral infections, the possibility exists for cross-family drug cocktails. Even a cocktail with incomplete suppression of viral replication could have a clinical benefit: for Ebola [68,69] and SARS-CoV-2 [166], a 1 log lower viral load has been associated with survival. Reductions in viral loads may also have public health benefits by reducing transmission rates and the opportunity for new variants.

### III. Methods of Modeling In Vivo Efficacy

High throughput multi-dose matrix assays are used to screen drug combinations with antiviral activity against Zika [96,97], arenaviruses [98], Ebola [22,23] and SARS-CoV-2 [86,99,100]. The results of those assays are reported in dose-response matrixes with efficacy of each combination reported within each matrix locus. To screen combinations based on the dose-response matrixes, there are available tools, such as SynergyFinder 2.0 [101,102] and MacSynergy II [103,104], which calculate synergy scores based on deviation in observed combined effects from expected efficacy of a drug combination given by different reference models including Highest Single Agent (HSA) [105], Loewe additivity [106], Bliss independence and Zero interaction potency (ZIP) [108]. Loewe additivity assumes drugs work via an equivalent mechanism with competition for the same binding site, while Bliss independence assumes that drugs work on different steps of the viral replication cycle generating multiplicative effects. Based on which reference model best explains the data, drug-drug interactions can be classified as antagonistic, Loewe additive, intermediate, Bliss independent or synergistic, among which synergy has the highest increase in combined efficacy, exceeding that of multiplicative effects [20]. The quantified synergy score demonstrates the strength of drug-drug interaction and measured biological effect.

However, despite the availability of these tools and models, many drug combinations do not provide sufficient antiviral activity when transitioned into animal models and/or human clinical trials or do not provide sufficient antiviral activity in vivo based on originally selected drug doses or dosing regimens. Mathematical modeling can be used throughout the development of combination antiviral regimens to maximize the likelihood of successful drug, dose, and dosing interval selection, as well as critical features of study design. The mathematical models described herein are predicated on the concept that PK/PD equations by themselves are not fully sufficient for forecasting trial outcomes.

Accordingly, in some embodiments, the presently disclosed subject matter provides a method of modeling the in vivo efficacy of a drug combination in treating a viral infection. The presently disclosed modeling method combines PK and PD models and/or data with models of viral and immune dynamics in the absence of therapy. More particularly, the presently disclosed modeling method assesses how single drugs and drug combinations affect viral dynamics under different assumptions of IC₅₀s. Thus, the present method accounts for typical underestimations of in vivo drug IC₅₀s based on in vitro data and takes into account immune responses to viral infection in the absence of treatment. Therefore, the method can provide a more realistic model of drug efficacy on viral infection in vivo and maximize the likelihood of successful choice of drug, dose, and dosing interval when planning animal studies and/or clinical trials, saving considerable time and money during the drug development process. In particular, it is believed that the presently disclosed method relates to the first instance where combining PK, PD, and viral dynamics models has been used to predict in vivo efficacy of drug combinations.

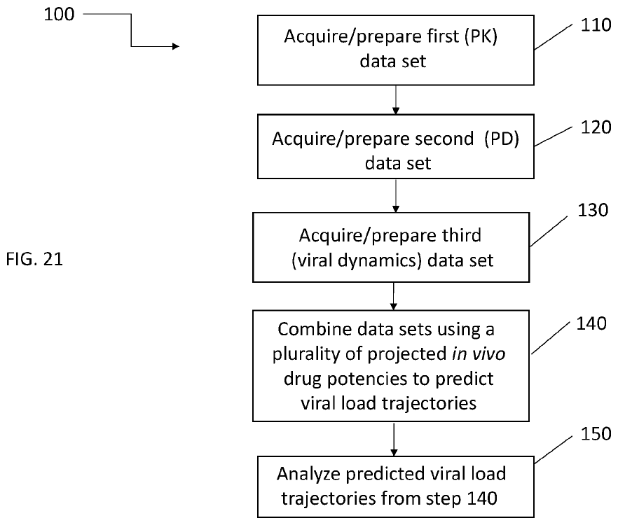

In some embodiments, the presently disclosed subject matter provides a method of modeling an in vivo efficacy of a drug combination in the treatment of a viral infection of a target virus in a target host subject. In some embodiments, the method comprises: (a) acquiring or preparing a first data set, wherein the first data set comprises pharmacokinetic (PK) data for a first drug and a second drug; (b) acquiring or preparing a second data set, wherein the second data set comprises pharmacodynamic (PD) data for the first drug, the second drug, and a combination of the first drug and the second drug; (c) acquiring or preparing a third data set, wherein the third data set comprises viral load dynamics data for the target virus in a relevant host subject; (d) combining the first, second and third data sets using a plurality of projected in vivo drug potencies against the target virus for each of the first drug, the second drug, and the combination of the first drug and the second drug, thereby predicting a plurality of different viral load trajectories in the target host subject; and (e) analyzing the plurality of different viral load trajectories predicted in step (d) to predict in vivo efficacy of the combination of the first drug and the second drug at one or more of the plurality of projected in vivo drug potencies. See FIG. 21, which shows exemplary method 100 including steps 110, 120, 130, 140, and 150, which correspond to steps (a), (b), (c), (d), and (e), respectively. Thus, FIG. 21 is a flow chart illustrating an exemplary process or method 100 for modeling the in vivo efficacy of a drug combination for treating or preventing a viral infections in an animal subject according to an embodiment of the subject matter described herein. In some embodiments, method 100 depicted in FIG. 21 comprises an algorithm and/or process stored in memory that when executed by a hardware processor of a computing device (e.g., a personal desktop or laptop computer, a tablet, a smartphone, a hand-held computer, a server, a mainframe, etc.) performs steps 110-150.

In some embodiments, the first drug and the second drug are orally available drugs previously approved for use for another indication (i.e., for treating a disease, disorder or condition other than treatment or prevention of infection of the target virus). The other indications can include, but are not limited to, cancer, hypertension, a psychiatric disorder, malaria, and bacterial or fungal infection. In some embodiments, the first and second drugs can be drugs that have been tested in vitro or in vivo in a subject other than the target host subject, separately or in combination. In some embodiments, the first and second drugs provide a combination suggested of having multiplicative or synergistic effect based on in vitro assay. In some embodiments, one or both of the first and second drugs can be different salt forms and/or structural analogs of drugs that have been previously shown to have antiviral effect, e.g., antiviral effect against the same or a different virus, either in vitro or in vivo. In some embodiments, the first and/or second drugs can target a viral glycoprotein, a viral polymerase, a viral protease, or can be directed against a target related to a pro-viral pathway (e.g., an inhibitor of host cell proteins that promote viral infection such as kinases and factors that promote virus entry replication, assembly, release and spread).

The target virus can be any virus or variant thereof. In some embodiments, the target virus is a virus that is newly causing infection in a particular population (e.g., humans, cows, sheep, etc.) or a virus causing newly increased levels of infection in a population and/or that is resulting in infections in a different or wider geographic range. In some embodiments, the virus is a common virus, such as, but not limited to, human papillomavirus (HPV), a herpes simplex virus, or an influenza virus. In some embodiments, the virus is a virus having high morbidity or mortality (e.g., an Ebolavirus species or Marburgvirus species or another virus that causes a viral hemorrhagic fever). In some embodiments, the virus is a virus or virus variant that is easily transmitted from one subject to another. In some embodiments, the virus is a virus that has the potential to be used in biowarfare (e.g., Variola (Smallpox) virus). In some embodiments, the virus is a respiratory virus, such as, but not limited to, an influenza virus, a parainfluenza virus, a rhinovirus, a respiratory syncytial virus, metapneumovirus, a coronavirus, an adenovirus, or a bocavirus. In some embodiments, the virus is selected from the group including, but not limited to, arenaviruses, bunyaviruses, calciviruses, filoviruses, flaviviruses, orthomyxoviruses, picornaviruses, rhabdoviruses, togaviruses, influenza viruses and coronaviruses. In some embodiments, the virus is a filovirus. In some embodiments, the virus is species of Ebolavirus or Marburgvirus.

The target host subject, i.e., a subject which the drug combination is being developed to treat or that is to be used for in vivo studies as part of a drug development program, can be any animal that can be infected by the target virus, such as a fish, bird, or mammal. In some embodiments, the target host subject is a human, a rodent (e.g., a mouse, rat, guinea pig or hamster), a mustelide (e.g., ferret), or a non-human primate. In some embodiments, the target host subject is a sub-population of a given animal. For example, the target host subject can be a human in a certain age range or a having a particular co-morbidity that can affect the host's response to viral infection. In some embodiments, the target host subject is an immunocompromised human. However, the target host subject can be any animal (e.g., mammal or bird) of importance due to being endangered (such as Siberian tigers), of economic importance (animals raised on farms for consumption by humans), and/or of social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Suitable avian target host subjects include those kinds of birds that are endangered, kept in zoos or as pets (e.g., parrots), as well as fowl, and more particularly domesticated fowl, for example, poultry, such as turkeys, chickens, ducks, geese, guinea fowl, and the like, as they are also of economic importance to humans. Thus, in some embodiments, the target host subject is an animal typically kept as livestock including, but not limited to domesticated swine (pigs and hogs), ruminants, horses, poultry, and the like.

The PK data acquired or prepared in step (a) can be data obtained/acquired from literature reports or can be prepared by newly performing PK studies of the drugs, e.g. in the target host subject. In some embodiments, acquiring or preparing the PK data can further comprise modeling PK data based on the data obtained from the literature or from performing PK studies using an available PK model, e.g., to stimulate or predict drug concentration levels in the blood or plasma of the target host subject over time based on projected drug dose(s) and/or route of administration. Thus, in some embodiments, the preparing of step (a) can comprise preparing a set of re-estimated data based on prior experimentally obtained data. In some embodiments, the PK data can include one or more of Cₘₐₓ, i.e., maximum (or peak) serum concentration (Cₘₐₓ) following administration; Tₘₐₓ, i.e., the time it takes the drug to reach Cₘₐₓ, Cₘₑₐₙ, i.e., the average (or mean) serum concentration of the drug following administration, tin, i.e., the time it takes for plasma concentration to decrease by 50%, Cₘᵢₙ, i.e., the minimum serum concentration; tₘᵢₙ, i.e., the time it takes the drug to reach Cₘᵢₙ, and MRT, i.e., mean residence time (MRT). Transfer of active drug metabolites between relevant tissue sites can be included if relevant data is available for model validation.

In some embodiments, acquiring or preparing a second data set (e.g., comprising (PD) data for the first drug, the second drug, and a combination of the first drug and the second drug) can comprise acquiring/obtaining literature PD data (e.g., of in vitro drug efficacy) or newly preparing data by performing in vitro drug efficacy assays (e.g., checkerboard assays of drug combination in vitro efficacy). In some embodiments, the PD data acquired or prepared can comprise data for IC₅₀, i.e., the concentration of drug required for 50% inhibition; a hill coefficient which is the slope of the dose response curve; or for a measure of drug combination synergy at two given drug concentrations. In some embodiments, step (b) comprises applying a PD model to PD data from in vitro or in vivo studies. In some embodiments, step (b) comprises fitting experimentally obtained in vitro or in vivo data to a mathematical model. In some embodiments, step (b) comprises modeling drug combinations at one or more sets (e.g., pairs) of doses of a drug combination, including one or more sets of doses for which no in vitro or in vivo experimental data is available. In some embodiments, step (b) comprises validating the PD mathematical model data against experimentally obtained in vitro or in vivo PD data. In some embodiments, step (b) comprises modeling drug combination interactions (e.g., synergy) across an entire drug concentration matrix for a drug combination of interest. In some embodiments, the PD model is a model based on a model such as, but not limited to HAS, Loewe additivity, Bliss independence, and ZIP, using an available tool, such as SynergyFinder 2.0 or MacSynergy II. In some embodiments, as described in the Examples below, the model is modified, e.g., by adding a power factor related to the percentage of infection events unaffected by both drugs. See Equation (2), in the Examples below. Thus, in some embodiments, IC₅₀ and/or hill coefficient from in vitro drug combination assays can be re-estimated across the entire range of possible drug concentrations.

Acquiring or preparing the third data set, can involve adopting or adapting a viral dynamics model or models from the literature or from new studies in a relevant host subject. Viral dynamics models describe the amount of free virus over time in a host subject. In some embodiments, the viral dynamics are based on data from an infected (but untreated) relevant host subject. By relevant host subject it is meant a host that is expected to have the same or similar viral dynamics to the intended target host subject. For example, for Ebola, data for viral dynamics models can be taken from studies done in non-human primates, as these are evolutionarily closely related to a typical target host subject, i.e., humans. In some embodiments, viral dynamics data is available for the target host subject and the relevant host subject is the same as the target host subject. In some embodiments, the viral dynamics model comprises data related to viral production and life cycle as well as viral inhibition related to the relevant host subject's cell defense mechanisms, innate immune response and adaptive immune responses.

As noted above, expected in vivo drug potencies are often underestimated when based on in vitro data. Thus, according to an aspect of the presently disclosed method, first, second and third data sets can be combined using a plurality of projected in vivo drug potencies (e.g., two, three, four, five or more projected in vivo drug potencies) against the target virus for each of the first drug, the second drug, and the combination of the first drug and the second drug to provide a plurality of different viral load trajectories in the target host subject (e.g., a prediction of viral load over time in the host subject under different treatment regimens). Each of the plurality of different viral load trajectories can relate to one of the projected in vivo drug potencies. For example, each projected in vivo drug potency (e.g., each 50% inhibitory concentration (IC₅₀) or 50% effective concentration (EC₅₀)) can be between about 0.1 times of the in vitro drug potency to about 10 times of the in vitro drug potency (e.g., about 0.1, 0.5, 1, 2, 3, 4, 5, 6, 7, 8, 9, or about 10 times of the in vitro drug potency). Combining the PK, PD, and viral dynamics can be performed, for example, using Equation 3 in the Examples below.

Analysis of the plurality of different viral load trajectories predicted in step (d) can predict the in vivo efficacy of the combination of the first drug and the second drug at one or more of the plurality of projected in vivo drug potencies. Thus, the analysis can provide information with regard to whether a modeled drug dose or dosing interval related to the drug combination can be expected to provide a level of viral inhibition suitable to control the virus as well as to provide information as to whether the drug combination provides more protection against the infection than the individual drugs used alone. Predictions of suitable treatment duration and suitable timing of treatment initiation can also be made. It is well within the scope of the presently disclosed modeling method to test different doses and dose timing. Thus, the method can be used quickly and flexibly test multiple doses, dose frequencies, combinations, treatment durations, and timing of treatment initiation. These predictions can be used to provide more accurate/advantageous drug combination selection, dose selection, and dosing regimen (e.g., dosing interval, number of doses, etc.) for in vivo animal studies and/or clinical trials.

In some embodiments, the method further comprises administering the combination of the first drug and the second drug to the target subject or a surrogate animal subject thereof (e.g., a non-human primate in the case of human target subjects), and the administering can comprise administering the combination of the first drug and the second drug at dose levels and/or dosing intervals predicted to be effective in vivo by the analyzing of step (e). The administering can be for therapy in a target subject in need thereof or for in vivo assessment in clinical or pre-clinical studies (e.g., in an animal model of disease). In some embodiments, the administering can be performed via oral or intranasal administration of the combination. However, all relevant dosing routes can be considered including, but not limited to intramuscular, intravenous, oral and inhaled. The first drug and the second drug can be administered in the same or different formulations and at the same or different times.

Generally, the earlier a treatment can be provided, the more likely it is that viral load can be controlled. However, in some cases, it might be expected that there would be a delay in providing treatment to a host subject who has been exposed to the virus. Thus, it can be helpful to understand the maximum time interval between virus exposure and initial treatment that will still provide effective viral control. In some embodiments, the analysis of step (e) comprises predicting the maximum time interval between initial exposure of a target subject to the target virus and an initial dose administration of the combination of the first drug and the second drug to the subject that provides a predetermined level of control of in vivo viral load. In some embodiments, the method further comprises administering an initial dose of the combination of the first drug and the second drug to the target subject or a surrogate animal subject thereof within the predicted maximum time interval.

As noted above, the degree of synergy between two drugs is often variable across different drug concentrations [109]. The combined efficacy can be increased in highly synergistic areas of a dose-response matrix, with less of an increase or even a decrease in other matrix regions. Thus, in some embodiments, the accuracy of PD modeling of a combination can be enhanced by more closely capturing concentration dependent drug-drug interactions. Because drug levels fluctuate over time in humans based on pharmacokinetic (PK) forces, more useful models are those that can more accurately predict efficacy of combined therapies over a wide range of dose combinations to optimize preclinical testing and clinical trials [110-113].

Classic reference models such as Loewe additivity and Bliss independence can fail to capture the full complexity of concentration dependent drug-drug interactions because they neglect certain areas within the drug combination matrix. Response-surface-based approaches, which assume Loewe additivity or Bliss independence, only partially capture concentration dependencies in drug-drug interactions [114,115]. The general pharmacodynamic interaction (GPDI) model evaluates a global interaction term to model efficacy of drug combinations with flexibility of interaction assumptions and examines directionality of drug-drug interaction by looking at how a drug affects PD parameters of the other one and vice versa [109]. The limitation of GPDI is that it is possible that a global interaction term over the dose-response matrix does not accurately recapitulate data because of non-linearity of concentration dependent interaction indexes [116]. The zero interaction potency (ZIP) model follows a similar logic as it calculates expected efficacy with unaltered PD parameters of the centric drug by another drug and quantifies delta scores based on observed efficacy and expectations for the interaction landscape surface [108]. ZIP then uses the interaction landscape surface to identify synergistic combinations but is not equipped to mathematically predict efficacy of all possible drug concentration combinations [108].

Accordingly, in some embodiments, the presently disclosed subject matter can involve a two-way pharmacodynamic (TWPD) modeling approach to capture concentration dependent drug-drug interactions and accurately recapitulate efficacy of drug combinations over the entire dose-response matrix. In some embodiments, the directionality of drug-drug interactions is assessed by switching the role (centric or secondary) of drugs and comparing the accuracy of alternative models. See Equations (4)-(6) in Example 7, below.

For instance, as described hereinbelow, the predictive power of TWPD was assessed by applying the approach to two repurposed anti-Ebola drug combinations (sertraline+bepridil) and (sertraline+toremifene). It was also applied to two repurposed anti-SARS-CoV-2 drug combinations (nitazoxanide+remdesivir and nitazoxanide+arbidol), the components of which include two broad-spectrum antiviral drugs (arbidol [72,117] and remdesivir [62,118,119]) and an antiprotozoal agent (nitazoxanide). It is demonstrated that TWPD can fully explain the PD of drug combinations across the dose-response matrix. The performance of TWPD is direction-independent indicating that whether a drug is considered centric or secondary does not affect model accuracy. Accordingly, in some embodiments, the method further comprises two-way concentration-dependent analysis of drug-drug interactions between the first drug and the second drug.

The presently disclosed method is not limited to modeling of drug combinations of two drugs. In some embodiments, the drug combination comprises at least one additional, i.e., third drug. Thus, in some embodiments, the first data set further comprises PK data for the third drug and the second data set further comprises PD data for the third drug, a combination of the first drug and the third drug, a combination of the second drug and the third drug, and a combination of the first drug, the second drug and the third drug. In some embodiments, step (d) comprises combining the first, second and third data sets using a plurality of projected in vivo drug potencies against the target virus for each of the first drug, the second drug, the third drug, the combination of the first drug and the second drug, the combination of the first drug and the third drug, the combination of the second drug and the third drug, and the combination of the first drug, the second drug and the third drug. In some embodiments, step (e) comprises analyzing the plurality of different viral load trajectories predicted in step (d) to predict in vivo efficacy of the combination of the first drug and the second drug, the first drug and the third drug, the second drug and the third drug, and the first drug, the second drug and the third drug at one or more of the plurality of projected in vivo drug potencies.

In some embodiments, one or more of the steps of the method can be performed using a processor (e.g., a hardware-based processor) and/or memory (e.g., for storing information and instructions to be executed by the processor). The processor can comprise any type of processor, such as a central processor unit (CPU), a microprocessor, a multi-core processor, and the like. Suitable memory can comprise one or more of random-access memory (RAM), read only memory (ROM), static storage such as a magnetic or optical disk, or any other type of machine or non-transitory computer readable medium. One or more steps of the method and indeed any steps, methods, processes, algorithms, and the like disclosed herein can be embodied by a module, which can be stored in memory and executed by a hardware processor.

### IV. Methods of Treating or Preventing Filovirus Infection

In some embodiments, the presently disclosed subject matter provides a method of treating or preventing diseases caused by viruses of the family Filoviridae (filoviruses). Filoviruses are negative strand RNA viruses that can infect humans and primates, as well as other animals, e.g., guinea pigs, marmosets, ferrets, mice, duikers, and bats. Filoviruses include viruses of the genera Ebolavirus and Marburgvirus. In particular, Ebola hemorrhagic fever is a severe, often-fatal disease in humans and has appeared sporadically since its initial recognition in 1976. The disease is caused by infection with an Ebolavirus. Six identified species of Ebolavirus are Zaire ebolavirus (the causative agent of Ebola ebolavirus), Sudan ebolavirus, Taï Forest ebolavirus, and Bundibugyo ebolavirus, each of which have caused disease in humans; Reston ebolavirus which has caused disease in nonhuman primates, but not in humans; and Bombali ebolavirus. Other undiscovered species can exist and are intended to be included in the scope of filoviruses.

Infection with an Ebola or Marburg virus usually causes life-threatening hemorrhagic fever. Symptoms include fever, severe headache, joint and muscle aches, chills, sore throat, weakness, nausea and vomiting, diarrhea, red eyes, raised rash, chest pain and cough, hiccups (Ebola virus), stomach pain, bleeding (from any bodily orifice), and psychological symptoms (confusion, irritability, aggression, or depression). As the illness progresses, jaundice, delirium, seizures, severe bleeding, organ failure, coma, shock, and death can occur.

Outbreaks of Zaire ebolavirus (EBOV) have been associated with high morbidity and mortality. Milestones have been reached recently in the management of EBOV disease (EVD) with licensure of an EBOV vaccine and two monoclonal antibody therapies. However, neither vaccines nor therapies are available for other disease-causing filoviruses. In preparation for such outbreaks, and for more facile and cost-effective management of EVD, the presently disclosed subject matter provides in some embodiments a cocktail containing orally available and room temperature stable drugs with strong activity against multiple filoviruses. More particularly, a vehicle for the component drugs of combinations of bepridil and sertraline and of sertraline and toremifene suitable for oral delivery to mice in a mouse model of EVD is identified; and it is shown that, thus formulated, the drugs are equally active against EBOV as preparations in DMSO, and that they maintain activity upon storage in solution for up to seven days. Pharmacokinetic (PK) studies indicated that the drugs in the oral delivery vehicle are well tolerated in mice at the highest doses tested. As described herein, mathematical modeling based on human oral PK, PD modeling, and viral dynamics projects that the combinations would be more active in humans than their component single drugs.

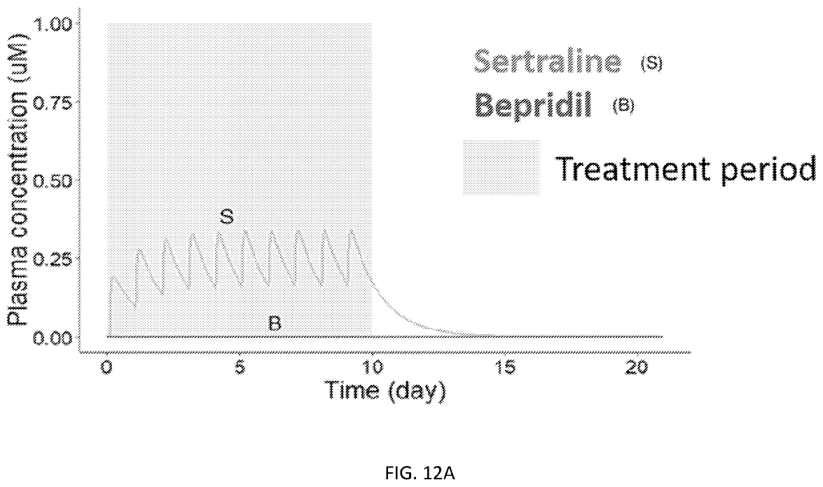

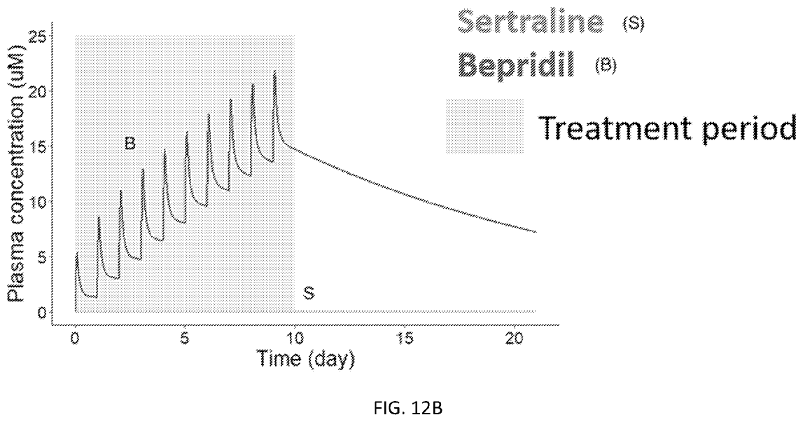

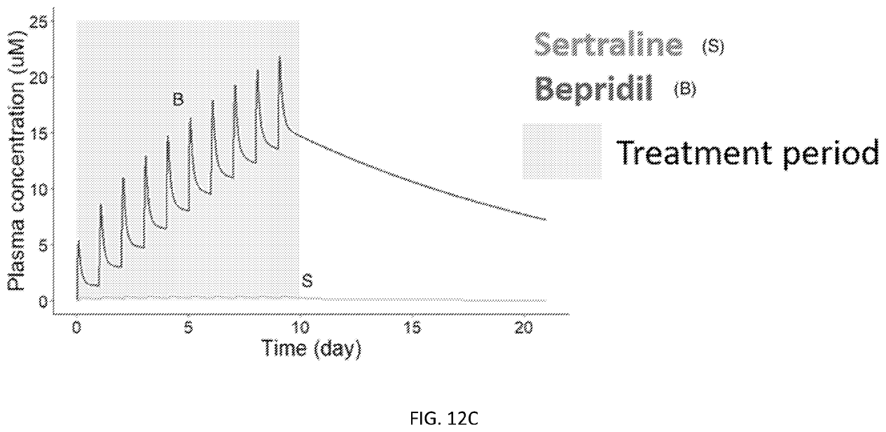

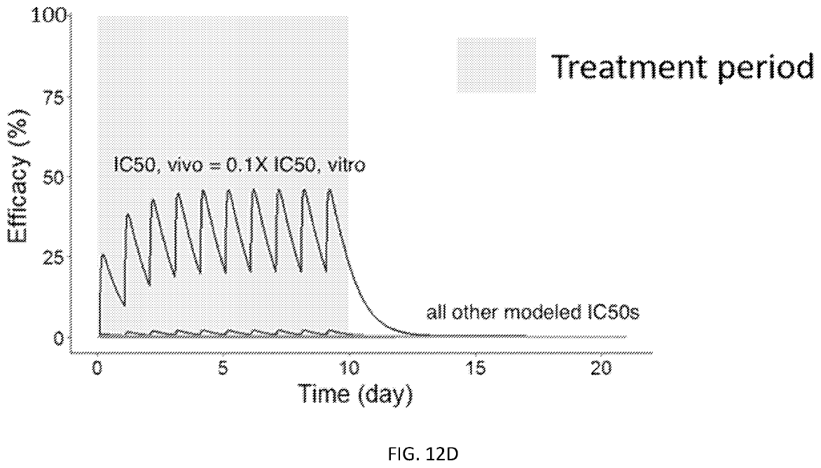

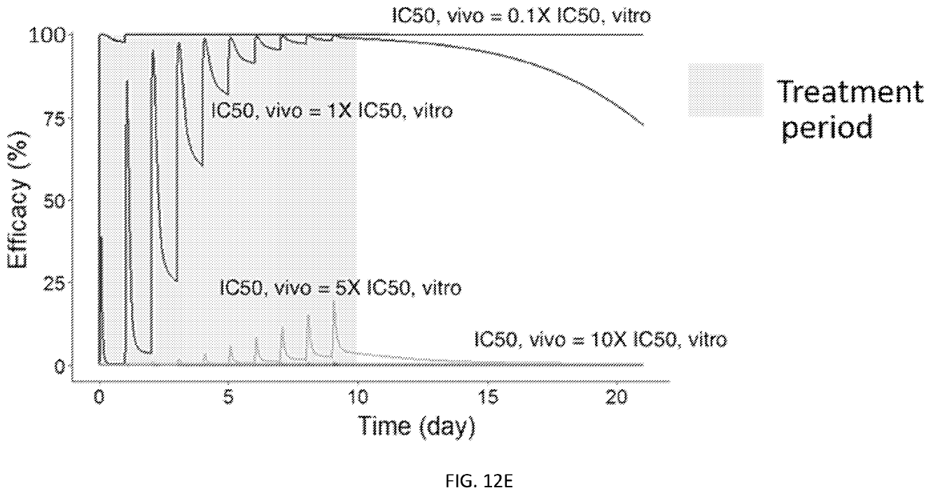

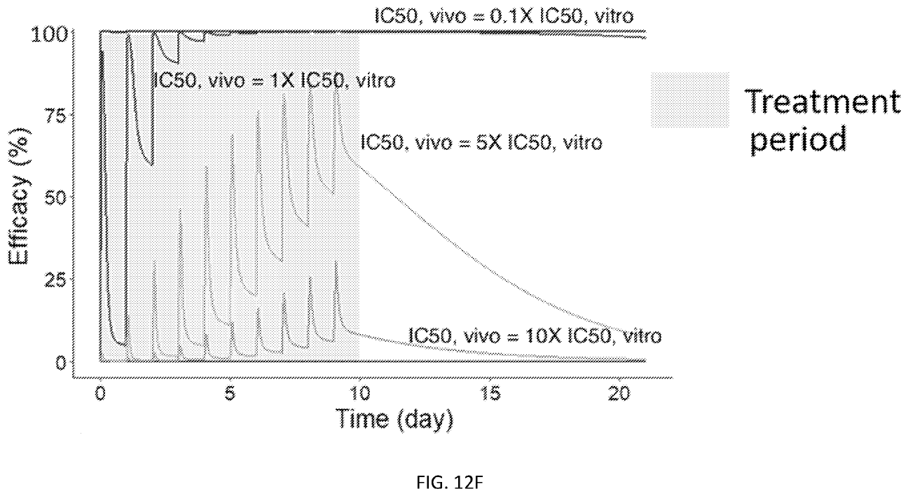

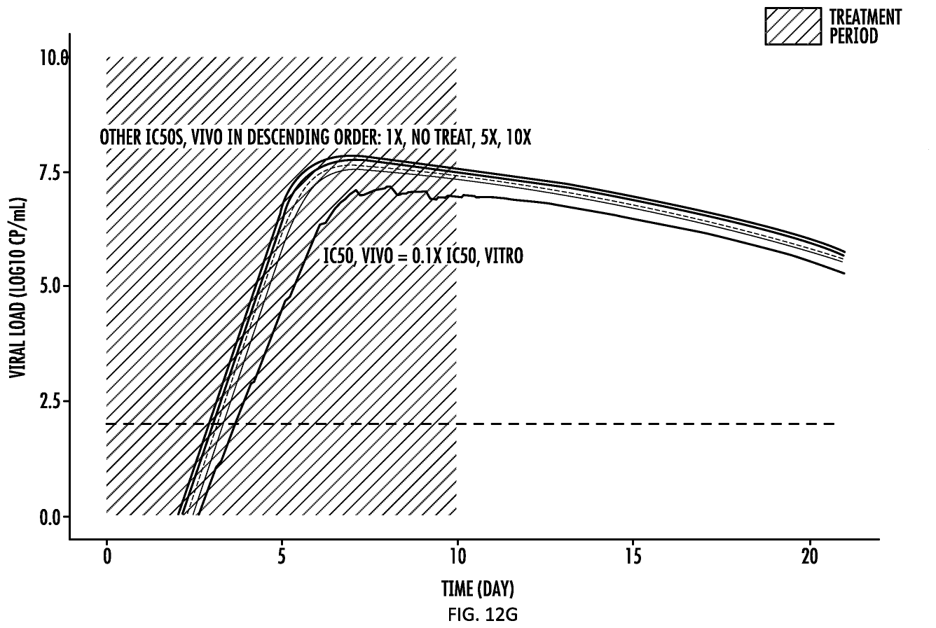

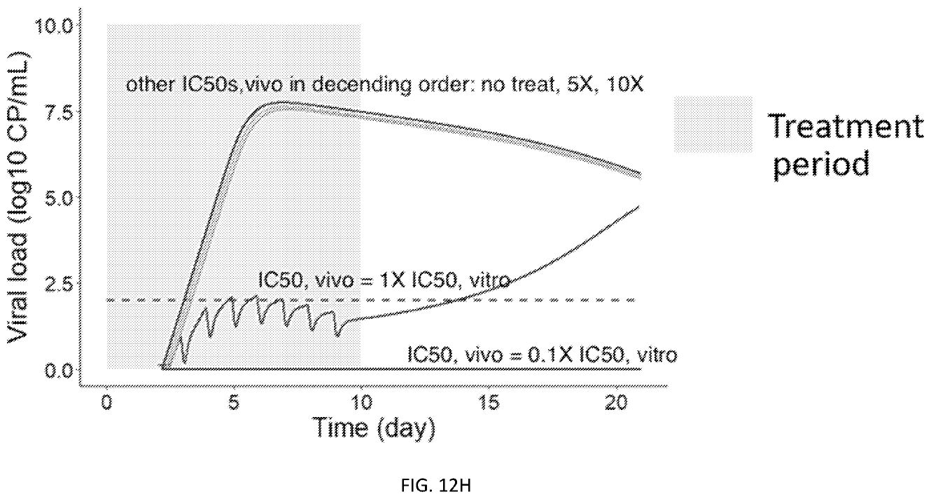

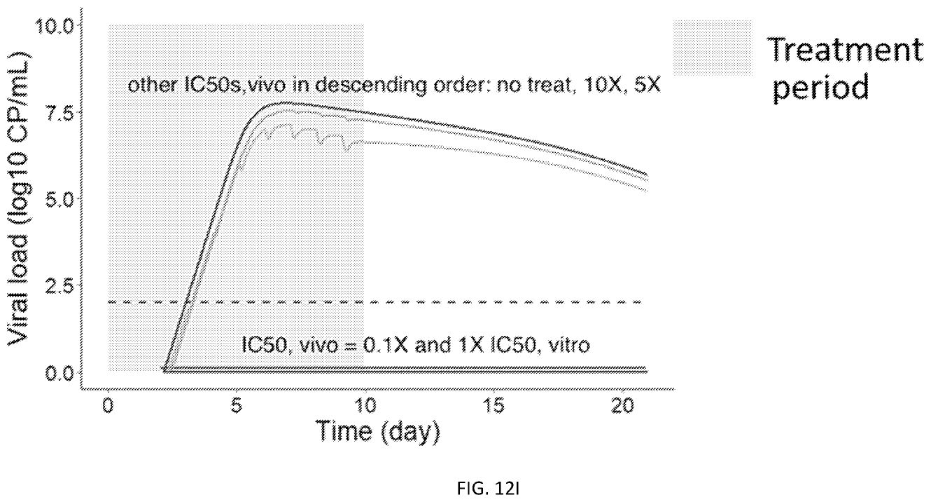

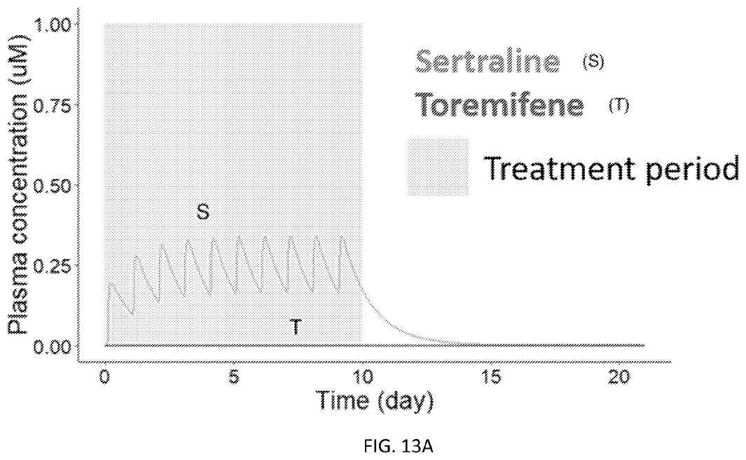

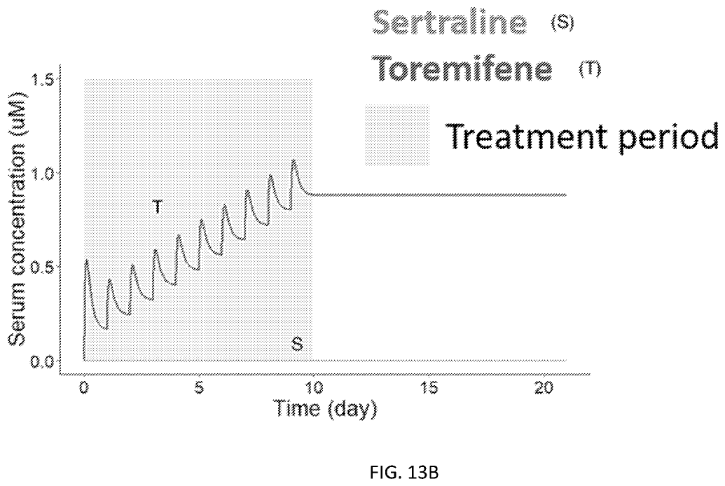

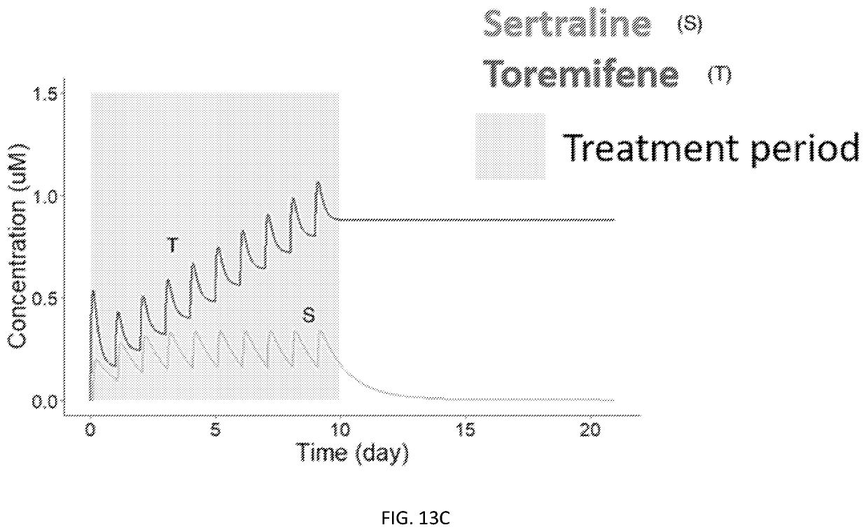

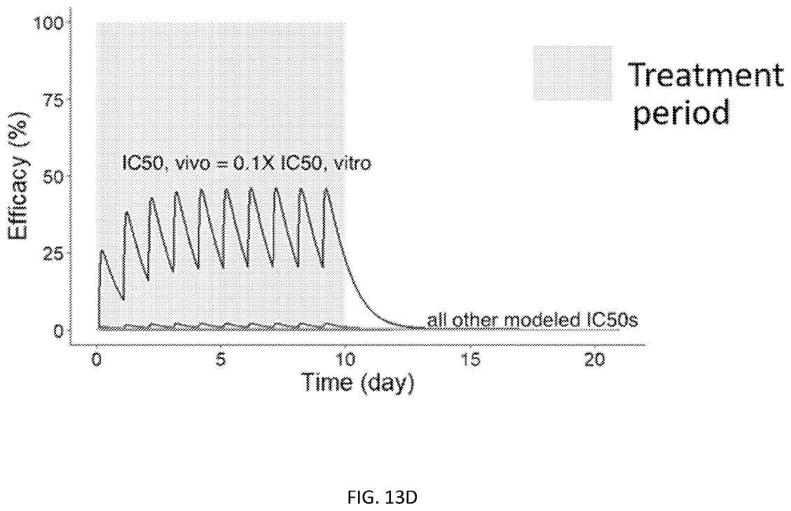

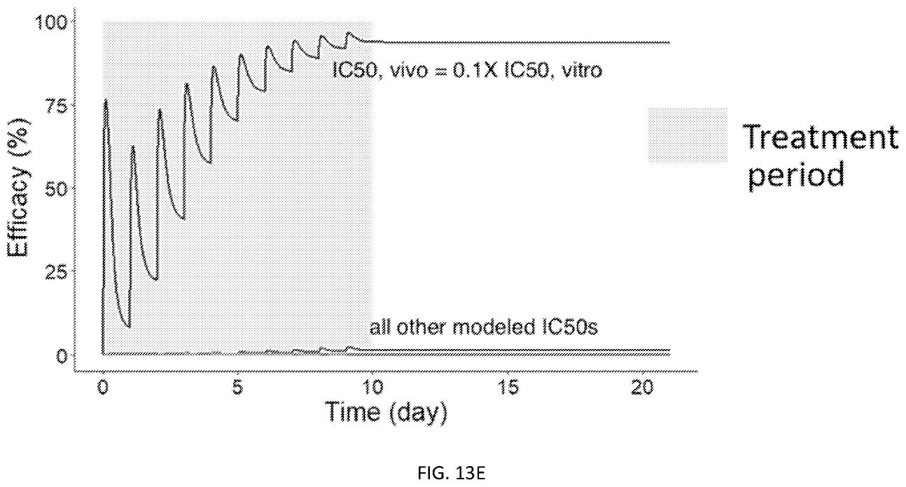

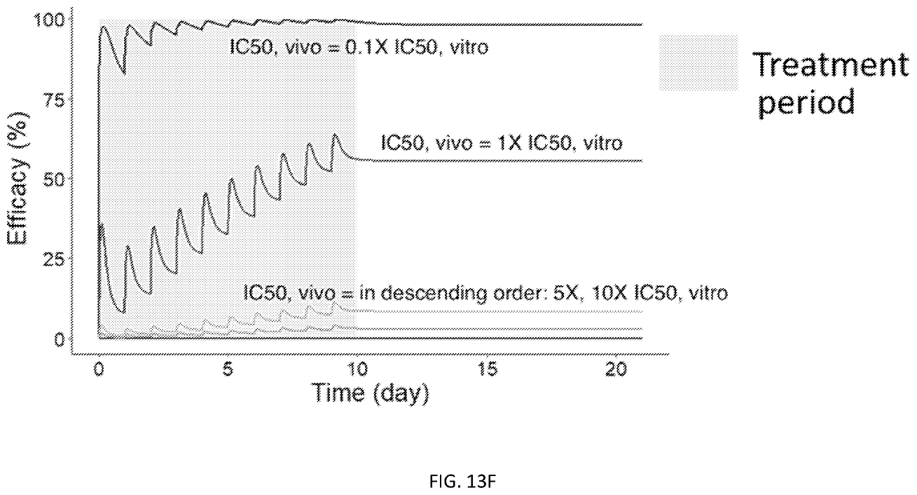

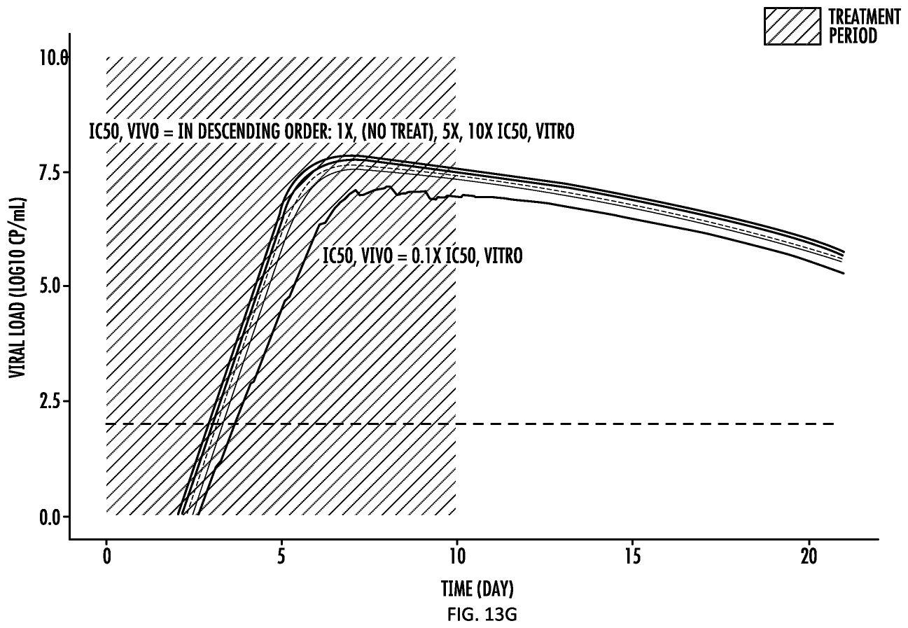

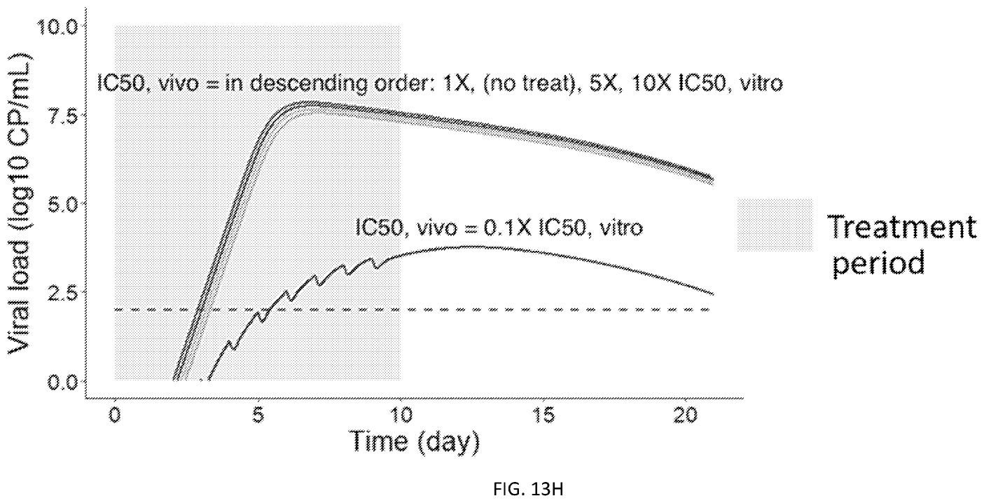

Accordingly, in some embodiments, the presently disclosed subject matter provides a method of treating or preventing a filovirus infection in an animal subject in need thereof. In some embodiments, the method comprising orally administering to said animal subject an effective amount of one of (a) a combination of bepridil or a pharmaceutically acceptable salt thereof and sertraline or a pharmaceutically acceptable salt thereof; or (b) a combination of sertraline or a pharmaceutically acceptable salt thereof and toremifene. In some embodiments, the method comprising orally administering to said animal subject one of (a) a combination of bepridil or a pharmaceutically acceptable salt thereof and sertraline or a pharmaceutically acceptable salt thereof such that the combination provides a dose of 300 milligrams per day (mg/D) of bepridil and a dose of 200 mg/D of sertraline; or (b) a combination of sertraline or a pharmaceutically acceptable salt thereof and toremifene or a pharmaceutically acceptable salt thereof such that the combination provides a dose of 200 mg/D of sertraline and a dose of 150 mg/D of toremifene. As disclosed, for example, in FIGS. 12I and 13I, these combinations are predicted according to the presently disclosed methods of modeling in vivo efficacy, to effectively control viral load in humans.

In some embodiments, the filovirus is an Ebolavirus species. In some embodiments, the Ebolavirus species is Zaire ebolavirus (now generally referred to as Ebola ebolavirus), Taï Forest ebolavirus, Sudan ebolavirus, Bundibugyo ebolavirus. Bombali ebolavirus or Reston ebolavirus. In some embodiments, the Ebolavirus species is Zaire ebolavirus.

In some embodiments, the filovirus infection is an infection of a Marburgvirus species. In some embodiments, the Marburgvirus species is Marburg marburgvirus. In some embodiments, the Marburgvirus species is Ravn marburgvirus.

In some embodiments, the administering is performed intranasally. In some embodiments, the administering is performed via oral, intravenous, subcutaneous, intramuscular, or ocular administration. The two drugs of the drug combinations can be formulated and administered in a single composition. Alternatively, the two drugs of the drug combinations can be formulated and administered in separate compositions.

In some embodiments, the animal subject is a mammal. In some embodiments, the animal subject is a human. In some embodiments, the animal subject is an animal subject who has been diagnosed with a filovirus infection. In some embodiments, the animal subject is an animal subject who has been exposed to others diagnosed with a filovirus infection and/or who has been exposed to others suspected of having a filovirus infection. In some embodiments, the animal subject is an animal subject who has traveled to an area with a high rate of filovirus infection of the pathogen. In some embodiments, the subject is a subject who has a filovirus infection, is suspected of having a having a filovirus infection (e.g., by exhibiting one or more symptoms of a filovirus infection, such as, but not limited to headache, muscle ache, weakness, fever, stomach pain, bleeding from an orifice, confusion or seizures), or is a subject who has one or more increased risk factors for a filovirus infection (e.g., by living in a geographic area or having traveled to a geographic area of a filovirus outbreak).

In some embodiments, the oral administration is first performed (i.e., a first daily dose is provided) within about 1 to five days (e.g., 1, 2, 3, 4, or 5 days) of exposure or suspected exposure of the subject to a filovirus that causes the filovirus infection. In some embodiments, the oral administration is first performed within about 48 hours (e.g., within about 2, 4, 6, 8, 10, 12, 14, 16, 18, 20, 22, 24, 26, 28, 30, 32, 34, 36, 38, 40, 42, 44, 46, or about 48 hours) of viral exposure or suspected viral exposure. In some embodiments, the oral administration is first performed within about 1 day (e.g., within about 24 hours) of exposure or suspected exposure to the virus.

In some embodiments, the administering is performed daily for about ten days or more (e.g., 10 to 15 days).

In some embodiments, the method further comprises administering one or more additional therapeutic agents to the animal subject. The one or more additional therapeutic agents can be an agent use to treat or mitigate one or more symptoms of the filovirus infections, such as a therapeutic agent for treating fever or pain. In some embodiments, the additional therapeutic agent can be an antiviral agent, e.g., an antiviral agent known in the field for treating a viral infection. The one or more additional therapeutic agents can be formulated and administered in the same composition as one or both of the drugs of the drug combination (e.g., bepridil and/or sertraline or sertraline and/or toremifene) or in separate compositions.

In some embodiments, the presently disclosed subject matter provides a pharmaceutical composition comprising a bepridil, sertraline, toremifene, or a combination of bepridil and sertraline or a combination of sertraline and toremifene. For example, the pharmaceutical composition can include a pharmaceutically acceptable carrier. In some embodiments, the compounds of the presently disclosed subject matter are formulated for use for oral administration, such as using a formulation known in the art for preparing bepridil, sertraline, or toremifene for treating another indication known to be treated by the drugs, e.g., depression, hypertension, or cancer. In some embodiments, the compounds can be formulated for oral administration in tablet, capsule or aqueous suspension form with conventional excipients for oral administration.

In some embodiments, bepridil, sertraline, toremifene, combinations of bepridil and sertraline or of sertraline and toremifene of the presently disclosed subject matter are provided for use in the treatment or prevention of filovirus infections, such as for use in the treatment of Ebolavirus or Marburgvirus infections, in humans and in animals. In some embodiments, separate compositions for the individual drugs can be provided for use in combination in the treatment or prevention of a filovirus infection. In some embodiments, a single composition comprising both drugs in the combination can be provided for use in the treatment or prevention of a filovirus infection. In some embodiments, a composition is provided comprising 300 milligrams of bepridil and 200 mg of sertraline. In some embodiments, a composition is provided comprising 200 mg of sertraline and 150 mg of toremifene. In some embodiments, said composition is formulated for oral administration. Thus, in some embodiments, the presently disclosed subject matter provides a pharmaceutical composition for use in treating or preventing a filovirus infection, said composition comprising: 300 milligrams (mg) of bepridil and 200 mg of sertraline, wherein the bepridil and the sertraline are formulated for oral administration separately (i.e., as two dosage forms to be used to treat the same subject on the same day) or in combination (i.e., in a single dosage form); or 200 mg of sertraline and 150 mg of toremifene, wherein the sertraline and the toremifene are formulated for oral administration separately or in combination.

Accordingly, in some embodiments, a composition for use can be provided wherein the composition is provided in a dosage form wherein a combination preparation comprising a mixture of bepridil or a pharmaceutically acceptable salt thereof and sertraline or a pharmaceutically acceptable salt thereof, is provided in a single dosage form. That is, a single dosage form comprising a mixture of bepridil or a pharmaceutically acceptable salt thereof and sertraline or a pharmaceutically acceptable salt thereof is provided. In the alternative a composition for use can be provided wherein the composition is provided in a dosage form wherein a combination preparation comprising a mixture of sertraline or a pharmaceutically acceptable salt thereof and toremifene or a pharmaceutically acceptable salt thereof, is provided in a single dosage form. That is, a single dosage form comprising a mixture of sertraline or a pharmaceutically acceptable salt thereof and toremifene or a pharmaceutically acceptable salt thereof is provided.

In some embodiments, a composition for use can be provided wherein the composition is provided in a dosage form wherein two separate individual preparations of bepridil or a pharmaceutically acceptable salt thereof and sertraline or a pharmaceutically acceptable salt thereof, are provided in two separate single dosage forms. That is, two separate single dosage forms are provided, a single dosage form comprising bepridil or a pharmaceutically acceptable salt thereof, and a single dosage form comprising sertraline or a pharmaceutically acceptable salt thereof. In some embodiments, a composition for use can be provided wherein the composition is provided in a dosage form wherein two separate individual preparations of sertraline or a pharmaceutically acceptable salt thereof and toremifene or a pharmaceutically acceptable salt thereof, are provided in two separate single dosage forms. That is, two separate single dosage forms are provided, a single dosage form comprising sertraline or a pharmaceutically acceptable salt thereof, and a single dosage form comprising toremifene or a pharmaceutically acceptable salt thereof.

### V. Pharmaceutically Acceptable Salts and Compositions

In some embodiments, the sertraline, bepridil, and/or toremifene of the presently disclosed methods of treating or preventing a filovirus infection or one or more of the drugs being modeled via the presently disclosed modeling method of in vivo efficacy can be provided as a pharmaceutically acceptable salt. Such salts include, but are not limited to, pharmaceutically acceptable acid addition salts, pharmaceutically acceptable base addition salts, pharmaceutically acceptable metal salts, ammonium and alkylated ammonium salts, and combinations thereof.

Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like.

Base addition salts include but are not limited to, ethylenediamine, N-methyl-glucamine, lysine, arginine, ornithine, choline, N, N′-dibenzylethylenediamine, chloroprocaine, diethanolamine, procaine, N-benzylphenethylamine, diethylamine, piperazine, tris (hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e. g., lysine and arginine dicyclohexylamine and the like.

Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

In some embodiments, the presently disclosed compounds can further be provided as a solvate.

Liquid carriers suitable for use in the presently disclosed subject matter include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellent.

Solid carriers suitable for use in the presently disclosed subject matter include, but are not limited to, inert substances such as lactose, starch, glucose, methylcellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins.

Oral formulations can include standard carriers such as pharmaceutical grades of mannitol, lactose, starch, magnesium stearate, polyvinyl pyrollidone, sodium saccharine, cellulose, magnesium carbonate, etc.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### Ebolavirus (EBOV) Cell-Based Infection Assay

EBOV infection assays were performed essentially as previously described [22,30]. In brief, for in vitro single agent drug tests, Huh7 cells were plated at 30,000 cells/well in black clear bottom 96 well plates. 24 hours later they were pretreated with drugs for 1 hour and then infected with EBOV/Mak (Ebola virus/H. sapiens-tc/GIN/201 4/Makona-005 (GENBANK® accession no. KX000398.1) (#IRF0165) at a multiplicity of infection (moi) of 0.21. The drugs were tested in 8-point dose response curves with 2-fold serial dilutions. Each dose was run in triplicate (n=3), and each experiment was run on at least duplicate plates. After 48 hours, the cells were fixed in formalin, and stained for the EBOV VP40 protein (mouse antibody #BMDO4B007) followed by peroxidase labeled goat α-mouse IgG (H+L), followed by a chemiluminescent substrate as previously described [31]. Plates were then read using a Tecan plate reader (model M1000, Tecan Group Ltd., Männedorf, Switzerland). For in vitro drug combination tests, cells, plated as above, were pretreated for 1 hour prior to infection with a 6×6 matrix containing 2-fold serial dilutions of Drug 1 and Drug 2, and then assessed for effect on EBOV infectivity as described above for the single agent tests. For drug combination experiments, cytotoxicity was determined (in parallel black opaque 96-well plates) using assay sold under the tradename CELLTITER-GLO® Luminescent Cell Viability Assay (Promega Corporation, Madison, Wisconsin, United States of America), and each datapoint (infection and cell viability) was analyzed (in each experiment) with 3 replicates. All work with live EBOV was conducted in a biosafety level 4 (BSL4) facility. Sources and catalogue numbers for the drugs tested are provided in Table 1, below. Sources of cells and cell culture media components are as previously described [32].

TABLE 1
Drugs for Cell-Based Infection Tests.

| | | Catalog |
|---|---|---|
| Name | Supplier | Number |
| Amodiaquine | Sigma Chemical Corp. (St. Louis, Missouri, United | A2799-5G |
| | States of America) | |
| Apilimod | Axon Medchem, LLC (Reston, Virginia, United | 1369 |
| | States of America) | |
| Aripiprazole | Selleck Chemicals (Houston, Texas, United States | S1975 |
| | of America) | |
| Bepridil | Sigma Chemical Corp. (St. Louis, Missouri, United | B5016-100MG |
| | States of America) | |
| Favipiravir | Selleck Chemicals (Houston, Texas, United States | S7975 |
| | of America) | |
| Piperacetazine | U.S. Pharmacopeia (Rockville, Maryland, United | na |
| | States of America) | |
| Sertraline | Toronto Research Chemicals, Inc., (North York, | S280000 |
| | Canada) | |
| Toremifene | Selleck Chemicals (Houston, Texas, United States | S1176 |
| | of America | |

IC₅₀ values were calculated as follows: background values were subtracted and inhibition was measured as percent relative to untreated infected cells. Non-linear regression analysis was then performed using GraphPad Software (La Jolla, California, United States of America), and IC₅₀ values were calculated from fitted curves (log [agonist] vs. response [variable slope] constrained to remain above 0. Error bars of dose-response curves represent the standard deviation (sd) of three replicates.

### Example 2

### Vesicular Stomatitis Virus (VSV)-EBOV GP-Pseudovirus Infection Assay

VSV pseudoviruses encoding EBOV GP deleted for its mucin domain (GPA) were prepared as previously described [7,33,34], but using VSV helper virus deleted for its own G protein gene (VSVAG) and encoding Renilla luciferase, VSV-AG-Luc (gift of Dr. Robert Doms, University of Pennsylvania). VSV-GPA-Luc infection assays were then performed essentially as previously described [7,33,34]. In brief, HEK293T/17 cells (CRL-11268; American Type Culture Collection (ATCC), Manassas, Virginia, United States of America) were plated in opaque white 96 well plates at a density of 30,000 cells/well. The cell culture medium employed was high glucose Dulbecco's Modified Eagle Medium (DMEM) supplemented with 1% L-glutamine, 1% sodium pyruvate, and 1% antibiotic/antimycotic, all from Gibco Life Technologies (Carlsbad, California, United States of America), and 10% supplemented calf serum (SCS; Hyclone, GE Healthcare Bio-Sciences, Pittsburgh, Pennsylvania, United States of America). Eighteen hours later the cells were pretreated with the indicated concentrations of the indicated drug for 1 hour and then infected with an amount of VSV-GPΔ-luc pre-titered to yield 100,000 relative light units (RLU) in uninhibited samples. After 24 hours, cells were lysed and analyzed for Renilla luciferase activity using a luciferase assay system sold under the tradename RENILLA-GLO® luciferase assay (Promega Corporation, Madison, Wisconsin, United States of America) according to the manufacturer's instructions. Each datapoint was analyzed (in each experiment) with 3 replicates. Sources and catalogue numbers for the drugs tested are described above in Table 1.

### Example 3

### Study of Apilimod Against EVOB in the Mouse Model

The efficacy of Apilimod (Axon Medchem, LLC, Reston, Virginia, United States of America, cat. #2500) was tested in the C57BL/6 mouse model of EVD. Apilimod was prepared fresh in saline (0.9% NaCl Injection, USP, Baxter Health Care Corporation, Charlotte, North Carolina, United States of America) from powder each dosing day. Apilimod powder was weighed, aliquoted and stored at 4° C. one day prior to the study start.

Doses of 30 mg/kg or 44 mg/kg of apilimod mesylate (equivalent to 20.5 mg/kg or 30 mg/kg apilimod, respectively in 0.9% NaCl Injection, USP) were administered IP to female C57BL/6 mice (Charles River Laboratories, Frederick, Maryland, United States of America) once a day for 10 days, beginning approximately 4 hours before exposure to ma-EBOV (mouse-adapted Ebola virus/Mayinga (GENBANK® accession no. KY425637.1). Compound efficacy was measured by assessing percent survival in the apilimod treated groups relative to the vehicle (saline) control group. Mice (10 per group) were challenged IP with 246 plaque forming units (PFU) of ma-EBOV on study day 0. Mice were observed for a total of 28 days, and mice that achieved a clinical score of 3 were euthanized.

Criteria for clinical scoring are outlined in the most current Integrated Research Facility (IRF) Animal-Care-and-Use-Committee-approved Animal Study Proposal. Mice were housed in an accredited BSL4 animal facility (accredited by the Association for Assessment and Accreditation of Laboratory Animal Care (AAALAC)). All animal procedures were approved (approval number IRF-032E, Jan. 22, 2015) by the Animal Care and Use Committee of the National Institute of Allergy and Infectious Diseases (MAID), Division of Clinical Research, in compliance with the Animal Welfare Act regulations, Public Health Service policy, and the Guide for the Care and Use of Laboratory Animals recommendations.

### Example 4

### Drug Formulation and Mouse Pharmacokinetic (PK) and Tolerability Tests

Formulation and tolerability studies were performed for bepridil, sertraline and toremifene. Details on the sources of the drugs and vehicle components used in ten test formulations are provided in Tables 2 and 3, below.

TABLE 2
Sources for Drugs for Formulation Studies.

| Name | Supplier | Cat. # |
|---|---|---|
| Bepridil Hydrochloride | Sigma-Aldrich (St. Louis, | B5016 |
| | Missouri, United States of | |
| | America) | |
| Clomiphene Citrate Salt | Sigma-Aldrich (St. Louis, | C6272 |
| | Missouri, United States of | |
| | America) | |
| Toremifene Citrate | Carbosynth Ltd. (Compton, | FT28330 |
| | United Kingdom) | |
| Sertraline Hydrochloride | Toronto Research Chemicals | S280000 |
| | (North York, Canada) | |

TABLE 3
Sources of Vehicle Components for Formulation Study.

| Name | Supplier | Cat. # |
|---|---|---|
| Benzyl alcohol | Sigma-Aldrich (St. Louis, Missouri, | 305197 |
| | United States of America) | |
| Capmul MCM NF | ABITEC Corporation (Columbus, Ohio, | N/A |
| | United States of America) | |
| Captisol | Captisol (San Diego, California, United | N/A |
| | States of America) | |
| Carboxymethyl- | Spectrum Chemicals (New Brunswick, | CA194 |
| cellulose Sodium, | New Jersey, United States of America) | |
| High Viscosity | | |
| Ethanol | Spectrum Chemicals (New Brunswick, | EI068 |
| | New Jersey, United States of America) | |
| NMP (1-Methyl-2- | Sigma-Aldrich (St. Louis, Missouri, | 328634 |
| pyrrolidinone) | United States of America) | |
| PEG 300 | Spectrum Chemicals (New Brunswick, | PO108 |
| | New Jersey, United States of America) | |
| PEG 400 | Spectrum Chemicals (New Brunswick, | PO138 |
| | New Jersey, United States of America) | |
| Polysorbate 20 | Spectrum Chemicals (New Brunswick, | PO132 |
| (Tween 20) | New Jersey, United States of America) | |
| Polysorbate 80 | Spectrum Chemicals (New Brunswick, | PO138 |
| (Tween 80) | New Jersey, United States of America) | |
| Propylene Glycol | Spectrum Chemicals (New Brunswick, | PR130 |
| (PG) | New Jersey, United States of America) | |
| Sodium Chloride | Spectrum Chemicals (New Brunswick, | SO155 |
| | New Jersey, United States of America) | |
| Sterile Water for | Henry Schein Vet (Henry Schein Inc., | 002488 |
| Injection | Melville, New York, United States of | |
| | America) | |
| Solutol (Kolliphore | Sigma-Aldrich (St. Louis, Missouri, | 42966 |
| HS 15) | United States of America) | |
| Vegetable Oil (Crisco | The J. M. Smucker Company (Orrville, | |
| Pure Vegetable Oil) | Ohio, United States of America) | N/A |

Vehicle #1 (Aqueous Suspension Vehicle, ASV) was prepared as follows: Sodium chloride (9 mg/ml final concentration) and sterile water for injection (70% final volume) were stirred for 5 min until the sodium chloride was dissolved. Benzyl alcohol (0.9% final concentration, v/v) was added, and the mixture was stirred for 5 min until it was a clear solution. Tween 80 (0.4% final concentration, v/v) was then added, and the mixture was again stirred for 10 min until Tween 80 dissolved. Carboxymethylcellulose (5 mg/ml final concentration) was then added while stirring. The solution mixture was stirred for 5 min before water was added Q.S. (as much as needed) to bring to the final volume. The ASV was stirred for 5 min, then autoclaved for 30 min at 121° C., and allowed to cool to room temperature for 90 min. The ASV was stored refrigerated until use.

Vehicle #2 (20% Captisol) was prepared by mixing Captisol (20% w/v) with water (˜70% of final volume) using a magnetic stirrer for 10 min until all solid was dissolved, then water was added Q.S. to the final volume and the solution was mixed for an additional 5 min.

Vehicles #3 and 6 were prepared by mixing the listed individual vehicle components at the appropriate concentration on a magnetic stirrer for 5 to 10 min. Solutol was melted at 50-60° C. before use. 0.1% Tween 20 in water was prepared by mixing Tween (0.1% v/v) with water (99.9% v/v) on a magnetic stirrer for 10 min.

Formulations in Vehicle #1, 2, 3 and 6 were prepared by mixing the appropriate amount of test article in the vehicle to achieve the target concentration. The formulations were mixed using a vortex mixer for 0 to 1 min and sonication for 15 to 30 min, and then stirred on a magnetic stirrer overnight.

Formulations in Vehicle #4 (Vegetable oil) were prepared by mixing the appropriate amount of test article) in Ethanol (100% of final volume) using a vortex mixer for 1 min and sonication for 20 to 30 min, then an equal volume of vegetable oil was added, then the mixture was mixed using a vortex mixer for 2 min. The mixture was placed under a speedvac (a centrifuge with vacuum pump). The level of ethanol was checked every 30 min and the mixture was mixed using a vortex mixer for 30 seconds at that same time; the speedvac process continued for 1 hour until all of the ethanol was removed. This procedure produced a formulation of test article in vegetable oil at the desired concentration.

Formulations in Vehicle #5 (PEG 400) were prepared by mixing the appropriate amount of test article in PEG 400 using a vortex mixer for 1 min and sonication for 15 min, then stirring in a water bath at 40° C. for overnight.

Formulations in Vehicle #7-10 were prepared by step-wise addition of each vehicle component, in the order listed, to the appropriate amount of test article to achieve the target concentration. The formulations were mixed using a vortex mixer for 1 min and sonication for 15 to 30 min between the addition of each vehicle component until a clear solution or homogeneous suspension was obtained.

Tolerability studies were performed with bepridil, sertraline and toremifene prepared in Vehicle 8 (V8, which is 80% PEG 400/20% of 0.1% Tween-20 in Water) on female C57BL/6 mice (Charles River Laboratories, Hollister, California, United States of America) that were administered drugs orally (PO) for 11 consecutive says (once/day), followed by a 7-day recovery period. The preparation of bepridil, sertraline and toremifene in Vehicle 8 was as follows:
1. Prepare 0.1% Tween 20 in Water
   a. Measure 49.95 mL sterile water into a bottle
   b. Place water on magnetic stir plate,
   c. Place magnetic stirrer in water and turn on the stirrer.
   d. Measure out 0.05 mL Tween 20 and add to the water while stirring.
   e. Stir for 10 min
   f. Solution should be clear. Set aside at room temperature and continue to step 2.
2. Weigh out test articles (sertraline, bepridil and toremifene) into clear vials.
3. Add 0.8 mL PEG 400 to each tube of test article. Cap the vials.
   a. Bepridil: Vortex for 1 min then sonicate for 15 min
      i. Solution will be clear and slightly orange after this step
   b. Sertraline: Vortex for 1 min then sonicate for 15 min
      i. Solution will be suspension after this step
   c. Toremifene: Vortex for 1 min then sonicate for 15 min
      i. Solution will be a white suspension after this step
4. To the test article+PEG 400, add 0.2 mL of the 0.1% Tween 20 in water prepared in Step 1.
   a. Bepridil: Vortex 1 min
      i. Solution should be clear orange after this step
   b. Sertraline: Vortex for 1 min, then sonicate for 15 min
      i. Solution should be clear and colorless after this step
   c. Toremifene: Vortex for 1 min, then sonicate for 30 min
      i. Solution should be clear and colorless after this step

Unless stated, preparations of bepridil, sertraline and toremifene in Vehicle 8 were always prepared fresh on day 0 (d0) of a study and then stored at 4° C. Sertraline and toremifene were always usable directly from the refrigerator (i.e. no precipitates, not solid). In contrast, when removed from the refrigerator, bepridil was always solid (as if it had frozen) and contained precipitate. Three full cycles of the following three steps were always performed to resolubilize bepridil:
1. Incubate 5 min at 37° C.
2. Sonicate for 15 min
3. Vortex 1 min

Sonication was done using a bath sonicator (model #2510, Branson Ultrasonics, Brookfield, Connecticut, United States of America), which has no specific energy settings. No heat was applied during drug preparation. Once resolubilized, bepridil was used as soon as possible. If the bepridil re-solidified, the above three steps were repeated (2 or 3 full cycles) to get bepridil back into solution.

Pharmacokinetic (PK) studies were also performed. In brief, bepridil-HCl, sertraline-HCl and toremifene citrate were prepared in Vehicle 8 as described above. Single oral doses of bepridil (150 and 500 mg/kg), sertraline (30 and 60 mg/kg) and toremifene (100 and 200 mg/kg) were administered to female C57BL/6 mice (provided by BioLasco Taiwan, Taipei City, Taiwan) and analyzed for the indicated PK parameters according to standard procedures.

### Example 5

### Mathematical Modeling Studies

Human Pharmacokinetic Data:

Mean bepridil PK data were extracted from Wu et al. using WebPlotDigitizer (WebPlotDigitizer—Extract data from plots, images, and maps, Pacifica, California, United States of America), where 5 healthy male volunteers (2049 years old) were administered 400 mg bepridil-HCl orally. Mean toremifene PK data were extracted from Degregorio et al. using the same extraction method, where volunteers were orally administered 50 mg (n=3) and 100 mg (n=3) toremifene. Sertraline PK data were gathered from Ruderman [37], containing raw data for 14 healthy male volunteers (2840 years old) given a single oral dose of 100 mg sertraline (Teva-Sertraline 100 mg, Sertraline HCl).

Pharmacokinetic Modeling of Bepridil, Sertraline and Toremifene:

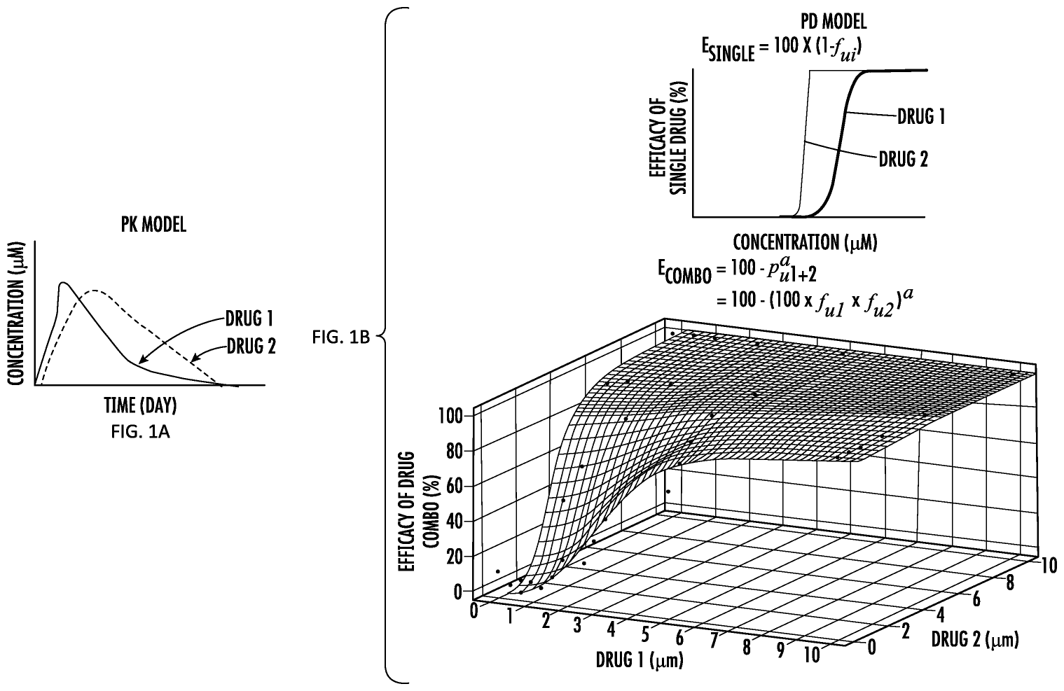

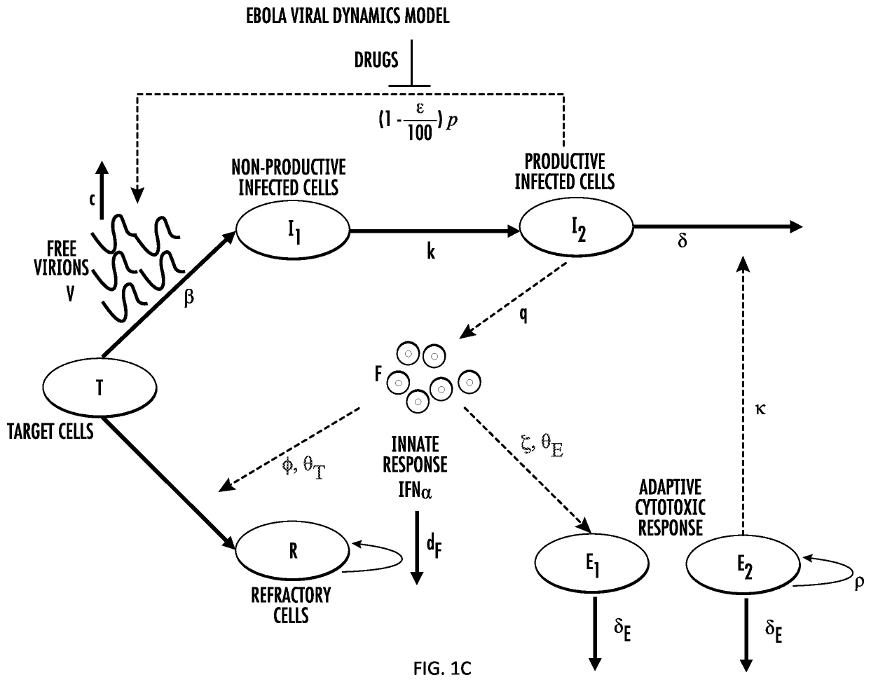

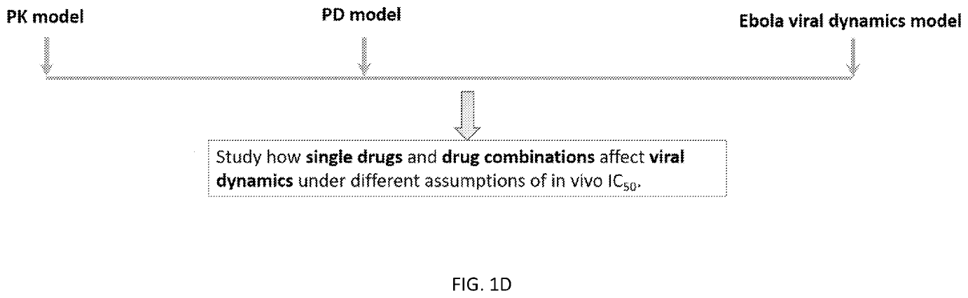

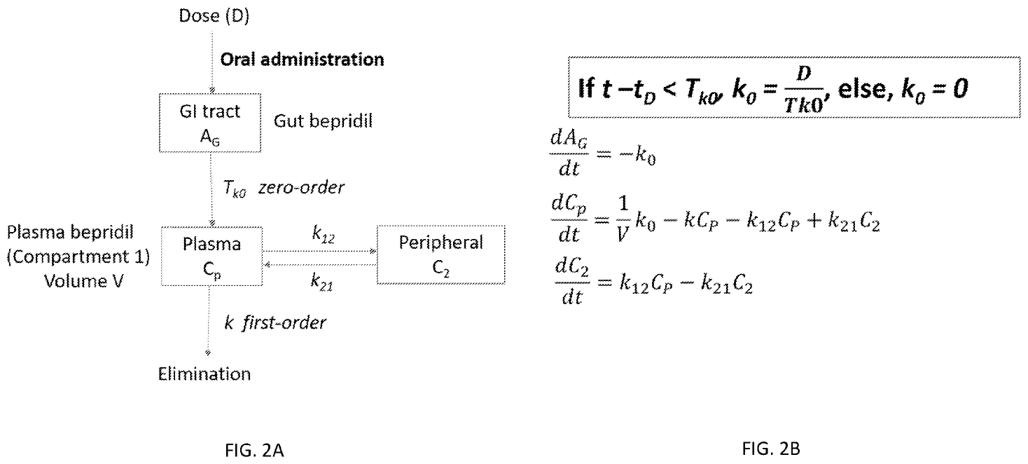

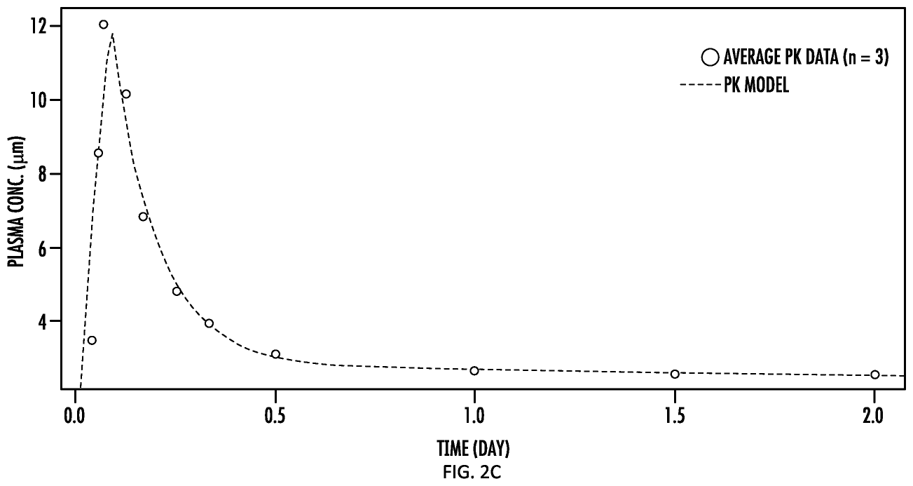

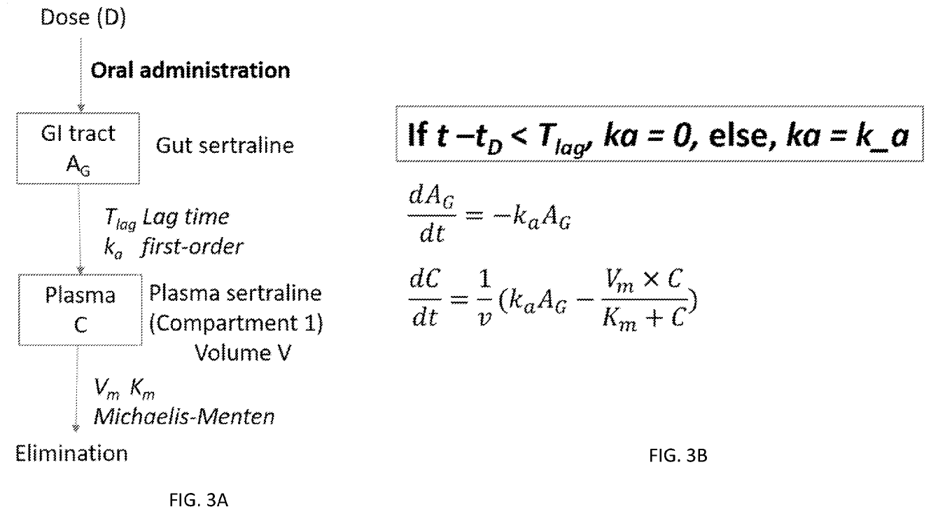

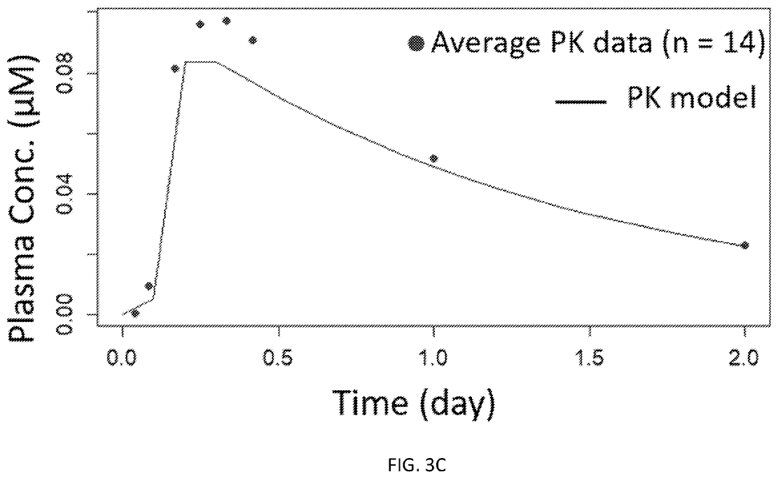

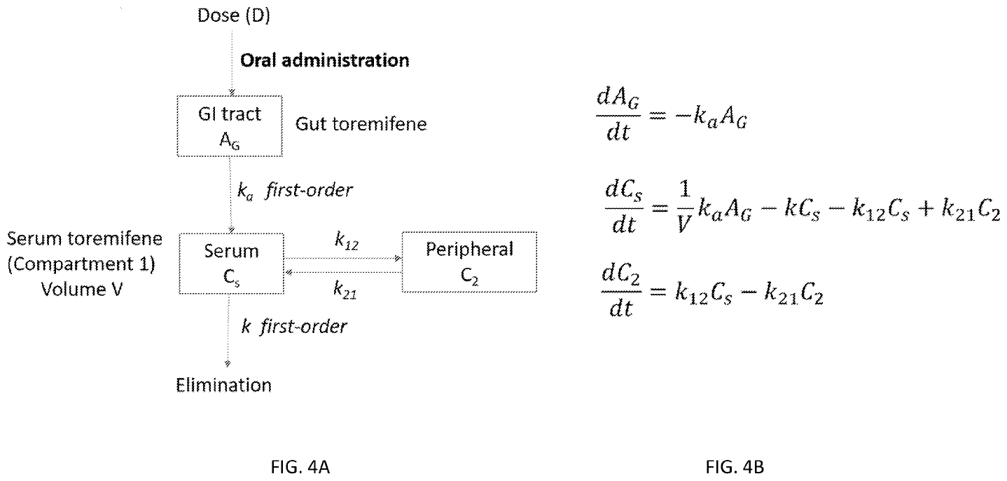

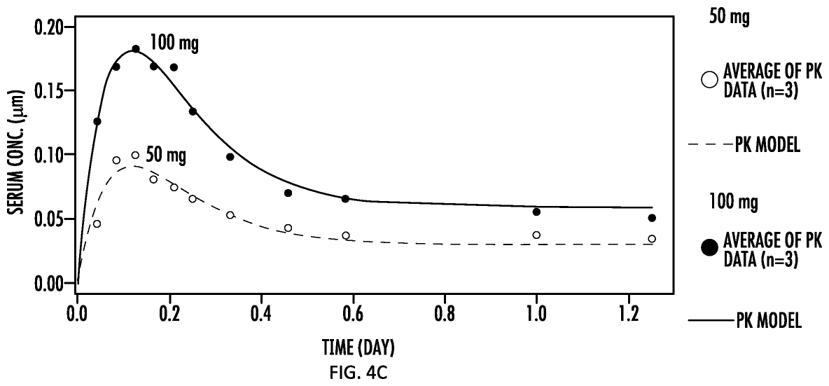

To model the PK of bepridil, sertraline and toremifene (see FIG. 1A), PK data (as mentioned above) was fitted to available PK models in Monolix [38]. The best PK model for each drug was selected that has the lowest Aikaike Information Criteria (AIC) score. See Tables 4-6, below, and FIGS. 2A-2C, 3A-2C, and 4A-4C. AIC rewards for close fit to data and penalizes for unnecessary complexity.

TABLE 4
PK parameters for bepridil.

| | |
|---|---|
| Tₖ₀ (day) | 0.0873 |
| V | 25.2 |
| k₁₂ (per day) | 6.71 |
| k₂₁ (per day) | 1.61 |
| k (per day) | 0.348 |

TABLE 5
PK Parameters for sertraline.

| | |
|---|---|
| T_{lag} (day) | 0.0976 |
| kₐ (per day) | 25.3 |
| V | 909 |
| vₘ (per day) | 2.61 × 10⁴ |
| kₘ (per day) | 32.2 |

TABLE 6
PK parameters for toremifene.

| | |
|---|---|
| kₐ (per day) | 16.6 |
| V | 354 |
| k₁₂ (per day) | 4.28 |
| k₂₁ (per day) | 0.992 |
| k (per day) | 0.000198 |

PD data of single drugs, bepridil, sertraline, and toremifene, as well as drug combinations of (sertraline+bepridil) and (sertraline+toremifene), were gathered from Dyall et al. [22]. The in vitro IC₅₀s and hill coefficients of bepridil, sertraline, and toremifene applied alone are from Dyall et al. and are reported in Table 7, below.

TABLE 7
In vitro PD parameters for single drugs. Data from [22].

| Drug | IC₅₀ (μM) | Hill coefficient |
|---|---|---|
| Bepridil | 5.86 | 4.859375 |
| Sertraline | 3.79 | 1.628 |
| Toremifene | 3.64 | 3.057 |

The efficacy of a single drug (%) equals:

E_{single}=100*(1−fᵤᵢ) Equation (1)

where fᵤᵢ is the fraction of infection events unaffected by drug i, that:

$${f}_{u i} = \frac{1}{1 + {( \frac{{D}_{i}}{{IC}_{50 , i}} )}^{{m}_{i}}}$$

Dᵢ is the concentration of drug i (μM). IC_{50,i} and mᵢ are the concentration of drug i (μM) required for 50% inhibition and the hill coefficient of drug i when drugs were used alone [20].

To model the efficacy of drug combinations, a pre-existing Bliss independence model [20] was modified by adding a power factor, a, to the percentage of infection events unaffected by both drugs (pᵤ₁₊₂), in order to capture the possible synergistic effects of drug combinations:

E_{combo}=100−pᵤ₁₊₂^{a}=100−(100×fᵤ₁×fᵤ₂)^{a} Equation (2)

Parameters of IC_{50,i} and mᵢ in the expression of fᵤᵢ were re-estimated in drug combination assays. See FIG. 1B.

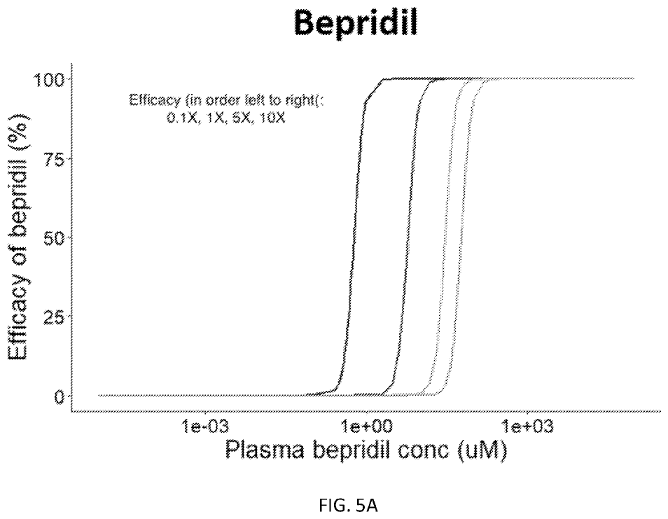

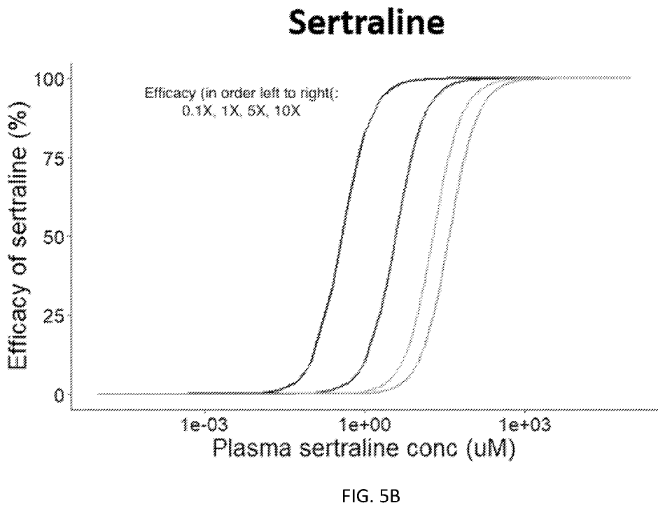

In vivo IC₅₀ was defined as the plasma drug concentration at which viral replication is inhibited by 50% in vivo [39,40]. Since in vivo IC₅₀s of bepridil, sertraline and toremifene in humans in single drug treatments and combined drug treatments are unknown, assumptions were made based on in vitro IC₅₀s measured in the dose response assays for the single drugs and drug combinations. More particularly, it was assumed that in vivo IC₅₀s are 0.1×, 1×, 5× and 10× of empirically observed in vitro IC₅₀s, and then pharmacodynamics of single drugs (see FIGS. 5A-5C) and drug combinations were predicted. PK and PD models were then linked to estimate percent of viral replication events inhibited as a function of time.

Mathematical Modeling of EBOV Viral Dynamics:

To study the possible advantage of drug combinations against EBOV infection, a previously built EBOV viral dynamics model [29], which is composed of viral production-inhibition, a cell defense mechanism mediated by IFNα, innate immune response and adaptive immune response, was used. See FIG. 1C.

It was assumed that drugs impact viral replication in the model according to drug plasma/serum concentration and PD curve according to the equation:

$$\frac{d V}{dt} = {\underbrace{( 1 - \frac{ε}{100} )}}_{\begin{matrix}Inhibition \\ of viral replication \\ by drug\end{matrix}} \times {\underbrace{{pI}_{2}}}_{\begin{matrix}Replication \\ of virions\end{matrix}} - {\underbrace{cV}}_{\begin{matrix}clearance \\ of virions\end{matrix}} \qquad Equation ( 3 )$$

where ε is the efficacy of a single drug (E_{single}, %) or the efficacy of a drug combination (E_{combo}, %), which is a function of the plasma/serum concentration of a drug (Equation (1)) or drugs (Equation (2)). Then, (1−ε/100) describes the fraction of inhibition on viral replication (pI₂) from reproductively infected I₂ cells, where p stands for production rate of virions (V). Virions have a clearance rate c. Thus, Equation (3) is the mathematical expression of the rate change of viruses (V).

Therefore, the EBOV viral dynamics model (FIG. 1C) comprises a system of ordinary differential equations [29]:

$$\begin{matrix}\frac{d T}{dt} = - β TV - \frac{ϕ T F}{F + {θ}_{T}} \\ \frac{{dI}_{1}}{dt} = β T V - {kI}_{1} \\ \frac{{dI}_{2}}{dt} = {kI}_{1} - δ {I}_{2} - κ {I}_{2} {E}_{2} \\ \frac{d R}{dt} = \frac{ϕ T F}{F + {θ}_{T}} \\ \frac{d V}{dt} = ( 1 - \frac{ε}{100} ) {I}_{2} p - c V \\ \frac{d F}{dt} = {qI}_{2} - {d}_{F} F \\ \frac{d L}{dt} = {q}_{L} {I}_{2} - {d}_{L} F \\ \frac{d N}{dt} = {q}_{N} {I}_{2} - {d}_{N} F \\ \frac{d {E}_{1}}{dt} = σ - \frac{ζ F {E}_{1}}{F + {θ}_{E}} - {δ}_{E} {E}_{1} \\ \frac{d {E}_{2}}{dt} = ρ {E}_{2} ( 1 - \frac{{E}_{2}}{{E}_{0}} ) - {δ}_{E} {E}_{2}\end{matrix}$$

### Example 6

### Discussion of Examples 1-5

Selection of Drugs for Combination Tests in Mice:

Seventy-eight pairwise combinations of approved drugs against EBOV in Huh7 cells were previously described [22]. Seven highly synergistic combinations are listed in Table 8, below. The second (bepridil+sertraline) and fourth (sertraline+toremifene) pairs for chosen for further study for the following reasons. The top pair (aripiprazole+piperacetazine) was excluded because piperacetazine is now only licensed for veterinary use in the United States of America (USA) and because in a first test (aripiprazole+piperacetazine) caused hypersomnolence in mice infected with EBOV, and thus malnutrition and dehydration likely contributed to the low (10%) survival rate [7]. The third pair (sertraline+clomiphene) was excluded because of poor human plasma exposure following oral administration of clomiphene (see Table 9, below) and variable survival rates in mice treated with clomiphene as a single agent. See Table 10, below. Combinations ranked as numbers 5-7 (apilimod paired with clomiphene, azithromycin, or toremifene) were also excluded. Although apilimod has potent activity against EBOV in cell cultures [32,41,42] and has a human Cₘₐₓ/IC₅₀>1 (see Table 9) suggesting potential activity in vivo, in a first test as a single agent in female C57BL/6 mice challenged with mouse-adapted EBOV (ma-EBOV), apilimod provided no survival advantage. See Table 9. Without being bound to any one theory, this was likely because apilimod is known to inhibit IL12/23 production [43,44], thereby interfering with IL12-mediated inhibition of EBOV infection (via interferon γ production) in macrophages [45], which are key targets of EBOV infection [2]. Hence, the components chosen for pairwise tests in mice were bepridil, sertraline and toremifene in the combinations (bepridil+sertraline) and (sertraline+toremifene).

TABLE 8
Highly synergistic drug pairs from Ref. [22],
rank ordered based on Matrix 3, Log Volume scores.

| | | Matrix 1 | Matrix 2 | Matrix 3 | Matrix 3 |
|---|---|---|---|---|---|
| Drug 1 | Drug 2 | (DBSumNeg) | (DBSumNeg) | (DBSumNeg) | (Log Volume ¹) |
| Aripiprazole | Piperacetazine | nd | nd | −5.14 | 28.0 |
| Bepridil | Sertraline | nd | nd | −3.06 | 17.2 |
| Sertraline | Clomiphene | −5.47 | −5.85 | −2.45 | 16.2 |
| Sertraline | Toremifene | −4.40 | −4.75 | −3.09 | 16.2 |
| Apilimod | Clomiphene | −4.90 | na ² | −1.79 | 10.2 |
| Apilimod | Azithromycin | nd | nd | −2.35 | 9.4 |
| Apilimod | Toremifene | −4.35 | na ² | −0.59 | 5.2 |

Drugs were tested against EBOV/Mak in Huh7 cells (moi 0.21) in three 6 × 6 checkerboard experiments: Matrix 1, Matrix 2, and Matrix 3 [22] (all data are available in Datasets 2 and 3, which accompany Ref. [22] as well as at: tripod.nih.gov/matrix-client. DBSumNeg is the sum of deviations from the Bliss model, a measure of synergy. Data from the Matrix 3 test were also analyzed with MacSynergy, which considers scores >9, Strong; >5, moderate; 2-5, minor; <2 insignificant.
¹ Log Volume scores are at the 99.9% confidence level.
² The apilimod used in this experiment did not have the correct chemical structure [22, 46].
na, not applicable; nd, not done.

TABLE 9
Predicted drug exposures (Cₘₐₓ) in humans following PO administration
compared to drug efficacy (IC₅₀) against EBOV in cultured liver cells.

| | IC₅₀ | IC₅₀ | IC₅₀ | Oral | Cₘₐₓ | | |
|---|---|---|---|---|---|---|---|
| | (Huh7) | (HepG2) | (average) | Dose¹ | (PO) | Cₘₐₓ/ | Reference |
| Drug | (μM) | (μM) | (μM) | (mg) | (μM) | IC₅₀ | PK Data |
| apilimod | 0.17 | nd | 0.17 | 70 | 0.26 | 1.56 | [43] |
| aripiprazole | 7.8 | 3.76 | 5.78 | 30 | 1.01² | 0.17 | [81] |
| azithromycin | 10.75 | nd | 10.75 | 500 | 0.64 | 0.06 | FDA |
| bepridil | 5.86 | 3.21 | 4.53 | 400 | 2.43 | 0.54 | [35] |
| clomiphene | 1.96 | 0.76 | 1.36 | 50 | 0.05 | 0.04 | [82] |
| piperacetazine | 7.58 | 3.30 | 5.44 | na | na | na | Na |
| sertraline | 3.79 | 1.44 | 2.62 | 200 | 0.54³ | 0.21 | [83] |
| toremifene | 3.64 | 0.03 | 1.83 | 60 | 1.97⁴ | 1.08 | [84] |

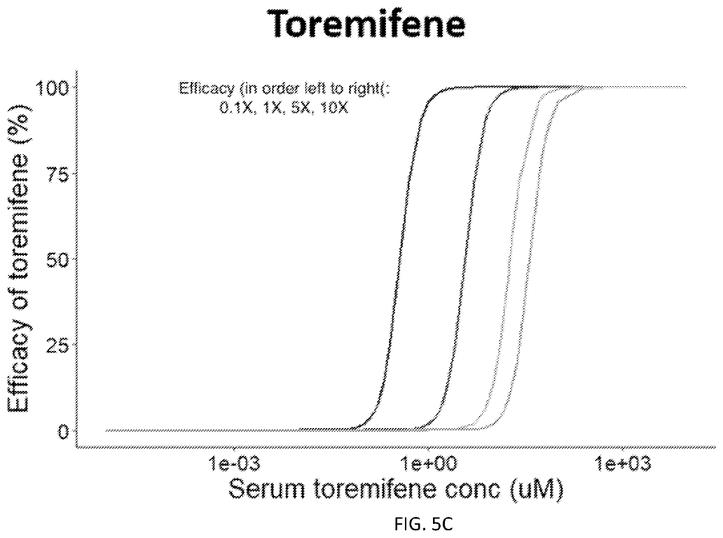

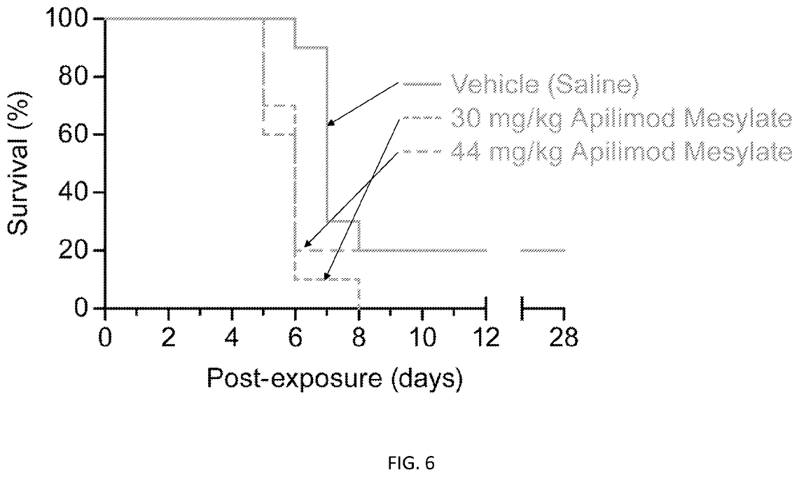

Values in column 2 are from Dyall ([22]; Dataset 1, all at moi 0.21; all from Sheet 4 of the Excel workbook except clomiphene and sertraline, which are from Sheet 2 of the Excel workbook). Values in column 3 are from Table 1 in Johansen [7]). Values in column 4 are the averages of the values in columns 2 and 3.
¹Cₘₐₓ after a single oral dose, unless specified.
²Cₘₐₓ after 14 days of daily dosing.
³Cₘₐₓ after 30 days of daily dosing to females (18-45 years of age); the 200 mg dose was reached after 3 days of dose increases starting at 50 mg.
⁴Cₘₐₓ after multiple days of daily dosing to healthy adults (see FIG. 6 in [84]). FDA refers to the FDA package insert for azithromycin.
Abbreviations: na, not available (piperacetazine is only authorized for veterinary use in the USA); nd, not done; PO, Per Os (by mouth).

TABLE 10
Literature values of drug efficacies as single agents
in mouse models of lethal EBOV infection.

| | Dose | | | Survival | |
|---|---|---|---|---|---|
| Drug | (mg/kg) | Regimen | Virus | (%) | Reference |
| Apilimod | 44 | IP, SID | EBOV | 0 | * |
| Aripiprazole | 20 | IP, SID | EBOV | 10 ¹ | [7] |
| Azithromycin | 100 | IP, SID | EBOV | 10, 30, 60 ² | [85] |
| Bepridil | 12 | IP, BID | EBOV | 100 ³ | [7] |
| | 12 | IP, BID | MARV | 90 ⁴ | [47] |
| Clomiphene | 60 | IP, QOD | EBOV | 90 ⁵ | [6] |
| | 60 | IP, BID | EBOV | 10 ⁶ | [85] |
| Piperacetazine | nd | nd | nd | nd | Nd |
| Sertraline | 10 | IP, BID | EBOV | 70 | [7] |
| Toremifene | 60 | IP, QOD | EBOV | 50 | [6] |

For the studies described in FIG. 6 and in References 7, 47 and 6, mice were treated on day 0 (d 0) and then on d 1-9 as indicated (either SID, BID, or QOD) and observed for a total of 28 days. For the study described in Reference 85, mice were treated on d 0 and then on d 1-7 and observed for a total of 14 days.
¹ Mice became extremely somnolent, thwarting eating and drinking, which likely contributed to low survival.
² 20% survival was seen in the control group in the study yielding 60% survival with azithromycin.
³ 90-100% survival has been seen dosing with 12 mg/kg, IP, SID (unpublished data).
⁴ In the same report, 80% mice were protected if treated with 12 mg/kg bepridil, IP, SID.
⁵ In the same report, a study comparing male and female mice (with fewer mice per group) yielded 60% and 40% protection of female and male mice, respectively (dosed with 12 mg/kg, IP, BID).
⁶ In the same report, no mice were protected if treated with 21 mg/kg clomiphene, PO, SID.
Abbreviations: BID, twice daily dosing; EBOV, Ebola virus; IP, intraperitoneal; MARV, Marburgvirus; nd, not done; QOD, dosing on d 0, 1, 3, 5, 7, 9. SID, once daily dosing.

Bepridil, sertraline and toremifene are orally available drugs (see Table 9) that block infections by EBOV and MARV in vitro [6,7,9]. Sertraline (Zoloft) and toremifene (Fareston) are prescribed in the United States of America and abroad for depression and metastatic breast cancer, respectively. While in use in several countries, Bepridil (Vascor) has been withdrawn as an antihypertensive in the United States of America. All three drugs showed efficacy via the intraperitoneal (IP) route in a mouse model of EBOV infection [6,7] (also see Table 10), and bepridil, which showed 100% protection in the mouse model [7], also strongly protected female Balb/c mice challenged with 195 PFU of ma-MARV Angola [47]. Sertraline was not effective at the dose employed in a test in rhesus macaques challenged with 1090 PFU of EBOV/Mak-005 [48]. Bepridil, sertraline and toremifene block EBOV entry into cells through late endosomes (LE) that bear the EBOV receptor, Niemann-Pick C1 (NPC1) [49-53]; they do not block EBOV binding to host cells, internalization from the cell surface or trafficking to NPC1⁺ LE [6,7,54]. Two co-functioning mechanisms likely account for their anti-EBOV action: (i) binding to a pocket in EBOV GP [55,56], with consequent effects on GP interaction with NPC1 [54] and conformational changes needed to elicit fusion [55-57], as well as (ii) disruptive effects on the composition and function of LEs [34,54]. Bepridil has also been reported to bind to two-pore calcium channel 2 (TPC2) [58], a host LE protein required for EBOV entry [59].

Selection of Drug Doses for Combination Tests in Mice:

For combination tests, an aim was to achieve doses resulting in 20-25% survival as single agents when given orally to mice enabling assessment of whether administration in pairs results in greater than 50% survival (i.e., more than an additive effect). Top doses of each drug (see Table 11) were chosen that should be safe in mice (based on LD₅₀) and achieve ≥50% survival, contemplating down-dosing (following a dose-down study) to achieve 20-25% survival as single drugs. The reasonings for the top dose of each drug are as follows (see Tables 9 and 10 for details). Bepridil: The aim is to achieve a Cₘₐₓ of ˜4 μM (average IC₅₀, see Table 9) to yield greater than or approximately equal to 50% survival. An oral dose (PO) of 150 mg/kg yielded a Cₘₐₓ of 1.5 04 [35]. Hence, 2.67-fold (4/1.5) more bepridil would be needed, suggesting a top oral dose of 400 mg/kg. As the oral LD₅₀ is 2069 mg/kg, top dose of 500 mg/kg was chosen. The Cₘₐₓ for the IP dose (12 mg/kg) of bepridil that yielded 100% protection was 3.3 μM. Sertraline: 10 mg/kg sertraline IP on a BID schedule (twice daily dosing) yielded 70% survival. See Table 10. It was approximated that daily dosing with 30 mg/kg would yield a similar level of protection. The oral Cₘₐₓ of sertraline (prepared in 0.9% saline) was ˜1/2 that obtained via the IP route. See Table 11. Hence, it was predicted that 30 mg/kg PO should yield −35% survival. Given an oral LD₅₀ of 336 mg/kg, 60 mg/kg was chosen as the top dose. Toremifene: 60 mg/kg toremifene (IP) on a QOD schedule (d0, d1 and then every other day for a total of six doses) yielded 50% survival. It was approximated that 30 mg/kg SID (once daily) would also protect ˜50% of infected mice. A comparison of PO (in 0.9% saline) to IP (in DMSO) administration indicated that the Cₘₐₓ for oral sertraline is 1/7 that seen via the IP route. See Table 11. It was reasoned that 30 mg/kg PO SID would yield ˜7% survival indicating a need for ˜7-fold higher dosing. Based on the oral LD₅₀ of 3000 mg/kg, 200 mg/kg was selected as the top toremifene dose (estimated to yield ˜50% survival).

TABLE 11
Rationale for choice of top oral doses.

| | IP Dose | | Survival | Oral Dose | | Oral Cₘₐₓ | Top Oral Dose | Oral LD₅₀ |
|---|---|---|---|---|---|---|---|---|
| Drug | (mg/kg) | Regimen | (%) | (mg/kg) | Oral Vehicle | (μM) | (mg/kg) | (mg/kg) |
| Bepridil | 12 | BID | 100 | 150 | Acacia | 1.5 | 500 | 2069 |
| Sertraline | 10 | BID | 70 | 10 | Saline | 0.43 | 60 | 336 |
| Toremifene | 60 | QOD | 50 | 10 | Saline | 0.16 | 200 | 3000 |

Mouse survival data are from references [6, 7].
Bepridil Cₘₐₓ is from reference [35].
Cₘₐₓ for sertraline and toremifene were determined for this study. BID, twice daily dosing (d 0-d 9); QOD, dosing d 0, d 1, 3, 5, 7, and 9.
Oral LD₅₀ (lethal dose for 50% of animals tested) data are from PubChem.
Bepridil and sertraline lethal dose values are for mice, and toremifene for rats.
The sertraline value was reported as LDLo (Lethal dose low, the lowest dose to have caused death).

Oral Formulation, Stability and Activity Tests of Bepridil, Sertraline and Toremifene:

The next step was to identify a vehicle compatible for PO delivery of all three drugs. For this purpose, we tested the solubility of bepridil, sertraline and toremifene in 10 vehicles. As seen in Table 12, below, none of the drugs were soluble in aqueous solution, but all three were soluble in Vehicles (V) 7-9. For further studies, V8 (80% PEG 400/20% of 0.1% Tween-20 in water) was selected. V8 was selected for further studies because it reproducibly formed a clear solution with all of the three test drugs and because it is comprised of more commonly available reagents (as compared with V7 and V9). Details for the preparation of V1-10 and stock solutions of bepridil, sertraline and toremifene are given above in Example 4. As noted there, bepridil was typically resuspended following storage in V8 at 4° C. or RT.

TABLE 12
Solubility of bepridil, sertraline, and toremifene in ten test vehicles.

| | Bepridil | Sertraline | Toremifene |
|---|---|---|---|
| Vehicle # | (solubility) | (solubility) | (solubility) |
| 1 | Suspension | Suspension | Suspension |
| 2 | Slightly hazy | Clear solution | Clear solution |
| 3 | Clear solution | Clear solution | Suspension |
| 4 | Clear solution | Clear solution | Non- |
| | | | homogeneous |
| 5 | Clear solution | Clear solution | Suspension |
| 6 | Clear solution | Clear solution | Suspension |
| 7 | Clear solution | Clear solution | Clear solution |
| 8 | Clear solution | Clear solution | Clear solution |
| 9 | Clear solution | Clear solution | Clear solution |
| 10 | Clear solution | Clear solution | Insoluble |

The vehicle formulations were: #1, Aqueous Suspension Vehicle; #2, 20% Captisol in water; #3, 37.5% PEG 400/37.5% Tween 20/25% Capmul MCM NF; #4, Vegetable Oil; #5, PEG 400; #6, 10% Solutol/90% PEG 400; #7, 5% NMP/95% PEG 300; # 8, 80% PEG 400/20% of 0.1% Tween-20 in Water; #9, 3% NMP/45% PEG300/12% ethanol/40% sterile water; #10, 40% Propylene Glycol/30% Solutol HS 15/30% Sterile Water. Drug concentrations were: Bepridil, 25 mg/ml; Sertraline, 3 mg/ml; toremifene, 8.4 mg/ml.

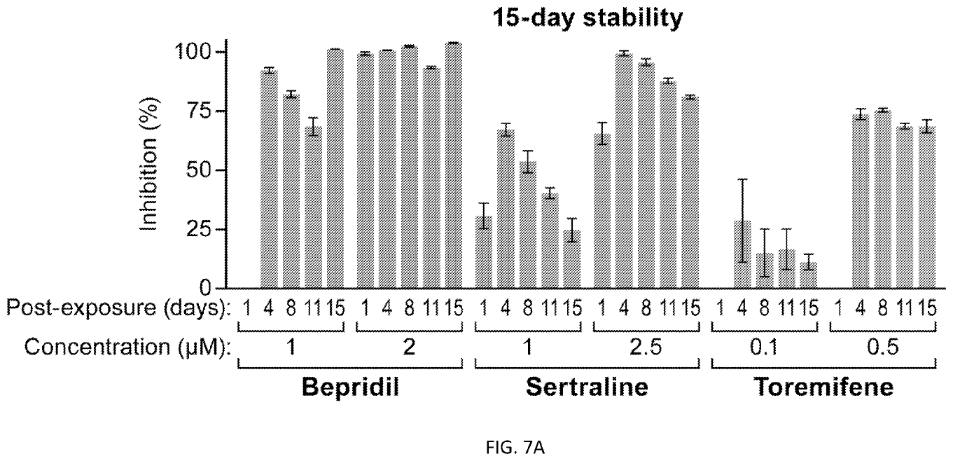

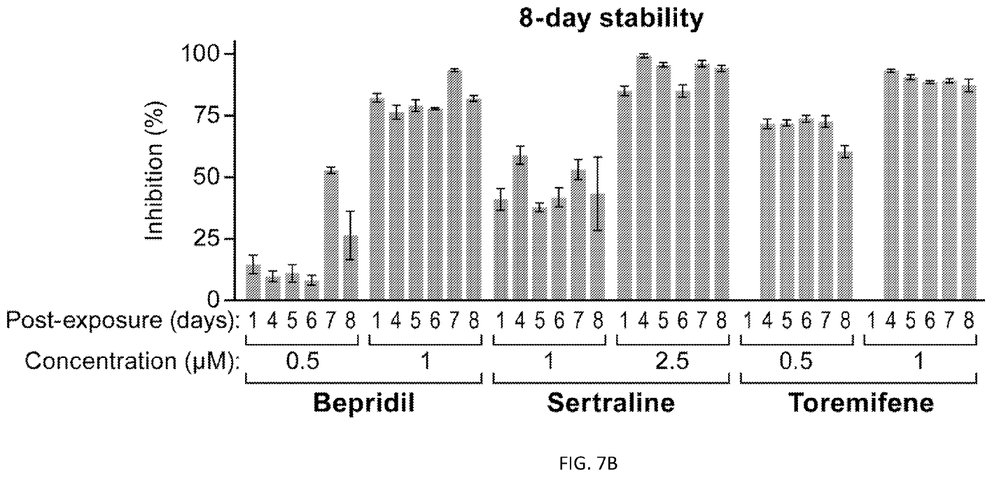

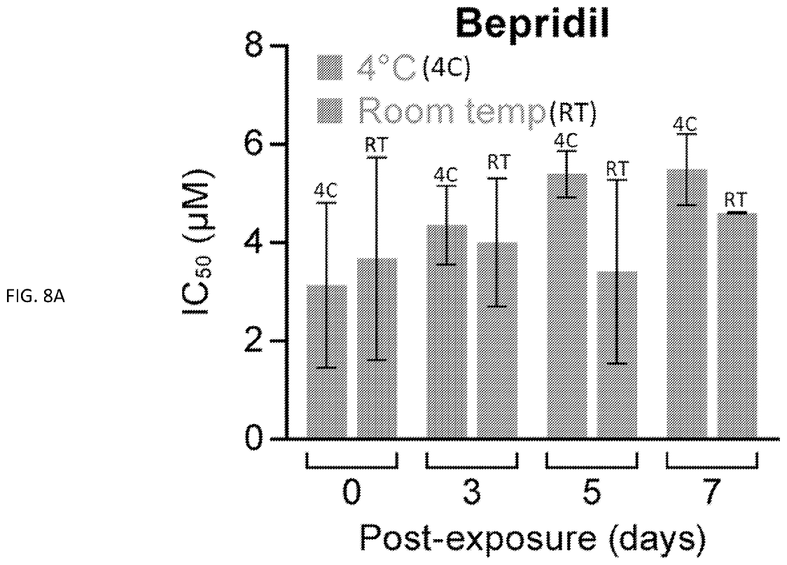

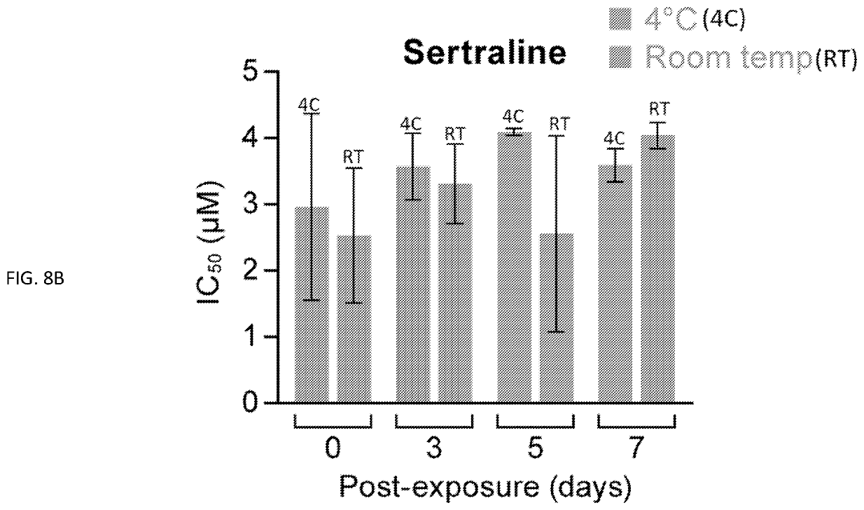

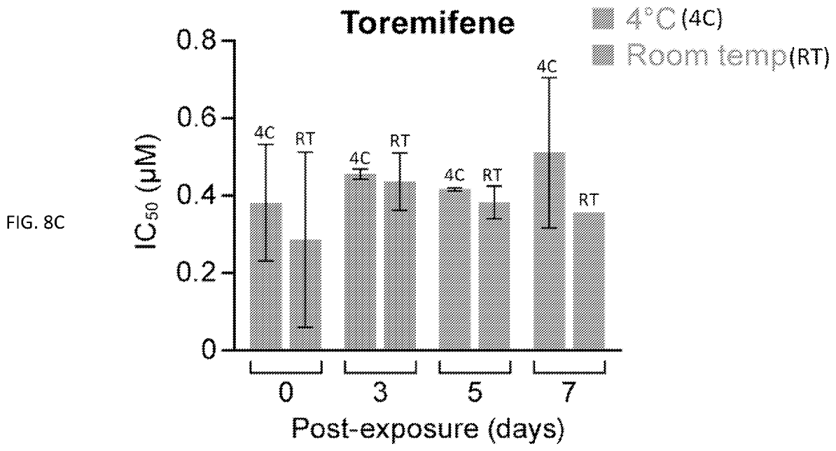

The stability of bepridil, sertraline and toremifene in vehicle #8 (V8) was studied. This was first performed using VSV pseudovirions bearing EBOV GPA (GP deleted for its mucin domain) and encoding Renilla luciferase (Luc), a system that can be used under BSL2 conditions to monitor EBOV entry [33,60,61]. In a first test, stock solutions in V8 were prepared and stored at 4° C. for 1-15 days. HEK293/T17 cells were pre-treated each day for 1 hr with the indicated concentration of the indicated drug. Cells were then infected with VSV-EBOV-GPΔ-Luc and infection scored 24 hours later based on the Luc reporter. As seen in FIG. 7A, whereas bepridil maintained inhibitory activity over 15 days, sertraline and toremifene began to lose activity after about 1 week. In a second experiment, all three drugs appeared stable in V8 over an 8-day period with possible small losses of activity between days 7 and 8. See FIG. 7B. These studies were extended to authentic EBOV (Makona isolate; EBOV/Mak), testing drugs in V8 stored at either 4° C. or RT for seven days. As seen in FIGS. 8A-8C, all of the drugs were stable for 7 days, with possible small loss of potency (<2-fold) for Bepridil stored at 4° C.

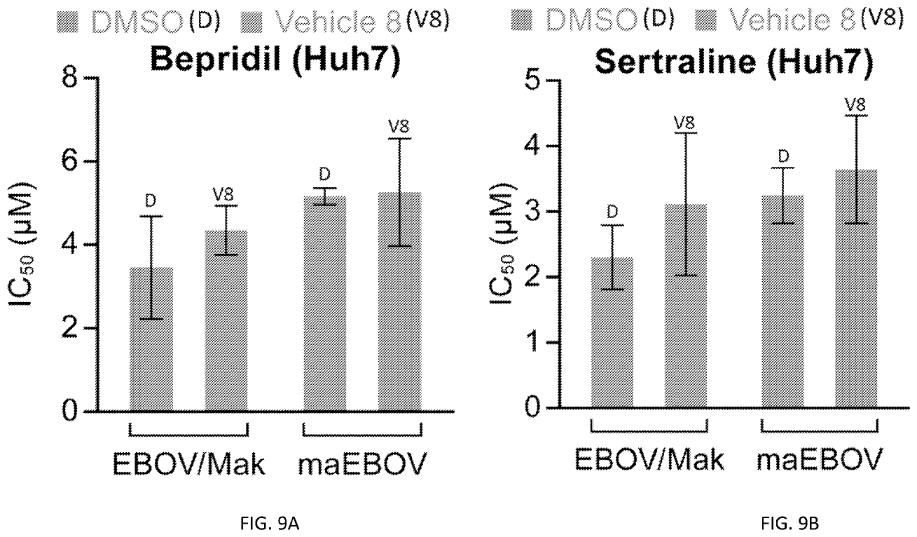

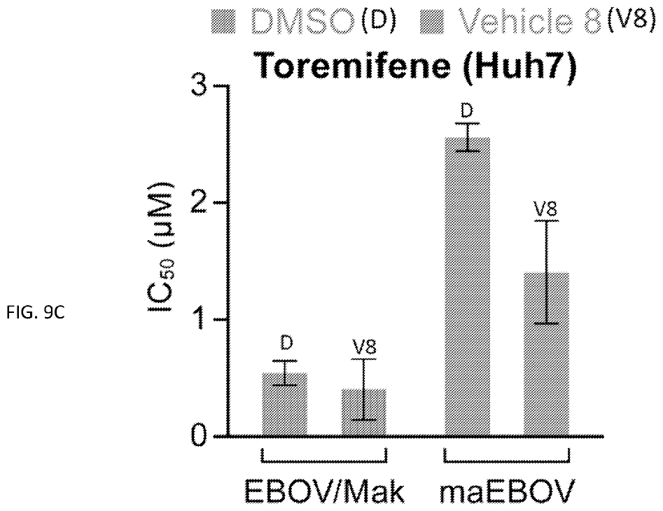

The potency of drugs stored for seven days at RT was assessed against EBOV/Mak and ma-EBOV, as the latter would be used for efficacy tests in mice. Similar potencies were seen for all three drugs whether prepared in DMSO or V8. Lower activity was noted for toremifene against ma-EBOV compared to EBOV/Mak, but this was seen for toremifene prepared in both DMSO and V8. See FIGS. 9A-9C.

Pharmacokinetic and Tolerability Tests of Reformulated Bepridil, Sertraline and Toremifene:

Having demonstrated solubility and defined stability of bepridil, sertraline and toremifene in V8, pharmacokinetic (PK) analyses were conducted in female C57BL/6 mice administered the drugs (in V8) by oral gavage. For each drug, the planned top dose and one lower dose were tested. The Cₘₐₓ, t_{1/2} and mean residence time (MRT) are given in Table 13, along with average IC₅₀s and consequent Cₘₐₓ/IC₅₀ values. The pre-PK predictions for Cₘₐₓ at the top doses (based on considerations described above) were: bepridil ˜3 NM, sertraline ˜1.2 NM, and toremifene ˜4.4 NM. The Cₘₐₓ values obtained (at the top doses) were near the predictions: ˜1.8- and 1.2-fold higher for bepridil and sertraline, respectively, and ˜1.4 fold lower for toremifene.

TABLE 13
Oral PK in mice following drug administration in V8.

| | Dose | Cmax | Cmax | MRT | T_{1/2} | IC₅₀ ¹ | Cₘₐₓ/ |
|---|---|---|---|---|---|---|---|
| Drug | (mg/kg) | (ng/mL) | (μM) | (h) | (h) | (uM) | IC₅₀ ¹ |
| Bepridil | 150 | 765 | 2.09 | 6 | 0.5 | 4.5 | 0.46 |
| | 500 | 1948 | 5.31 | 9 | 0.5 | | 1.18 |
| Sertraline | 30 | 188 | 0.61 | 11 | 1.0 | 2.6 | 0.23 |
| | 60 | 453 | 1.48 | 5 | 1.0 | | 0.56 |
| Toremifene | 100 | 498 | 1.23 | 6 | 0.5 | 1.8 | 0.67 |
| | 200 | 1295 | 3.19 | 16 | 0.2 | | 1.74 |

¹ average of IC_{50S} from two studies (see Table 9). MRT, Mean Residence Time.

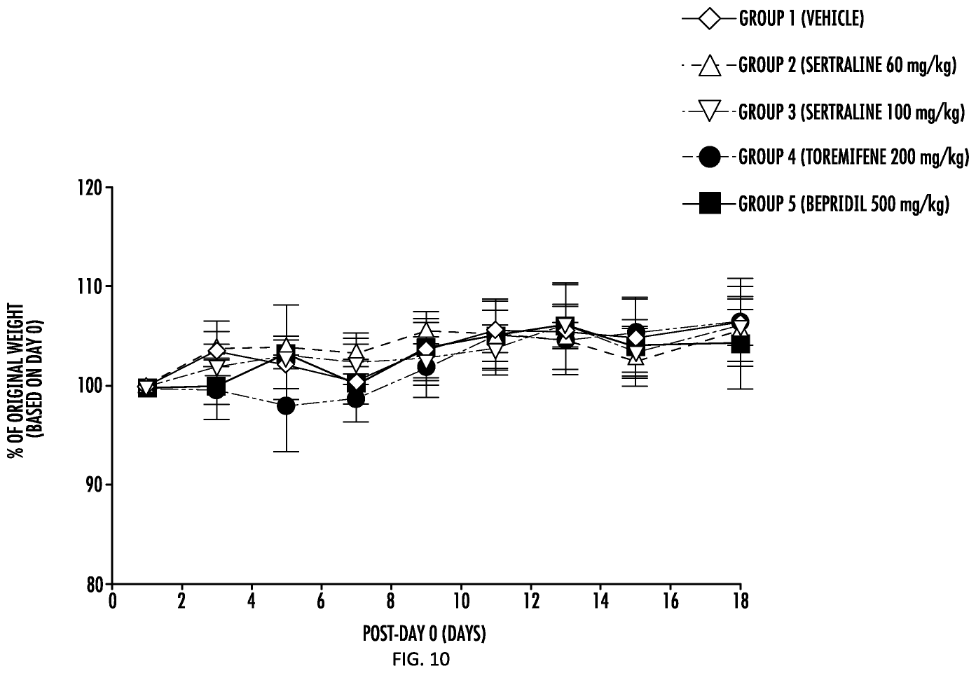

The tolerability of the three drugs was tested in V8, given once daily PO, for eleven consecutive days (drugs were freshly prepared in V8 every 5 or 7 days and stored at RT) in female mice that were weighed and observed for a total of 18 days. Bepridil and toremifene were administered at the top planned doses (500 and 200 mg/kg, respectively); sertraline was administered at the top planned (60 mg/kg) and one higher (100 mg/kg). As seen in FIG. 10, no aberrations in weight gain were seen. All mice survived, and no toxicologically significant changes were seen. Hence, bepridil, sertraline and toremifene are well tolerated in mice at the top target doses.

Mathematical Modeling Studies to Predict In Vivo Efficacy of Drug Combinations in Humans:

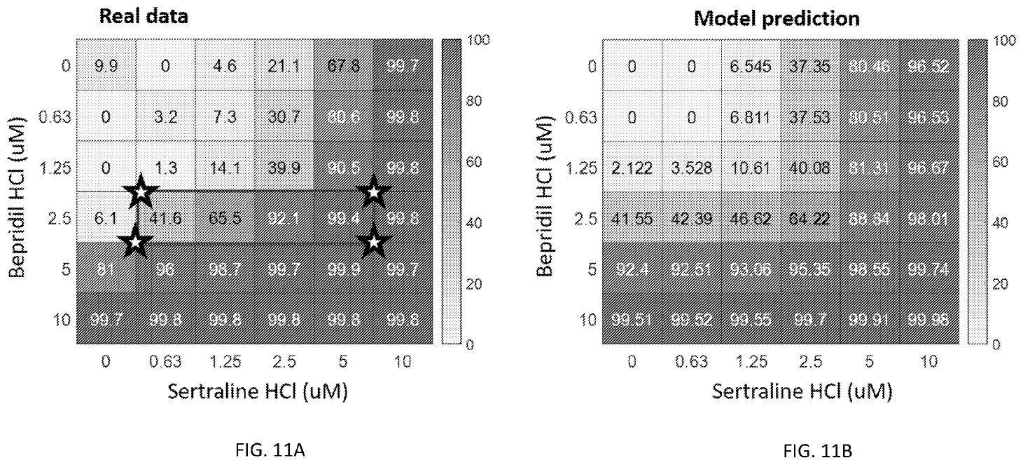

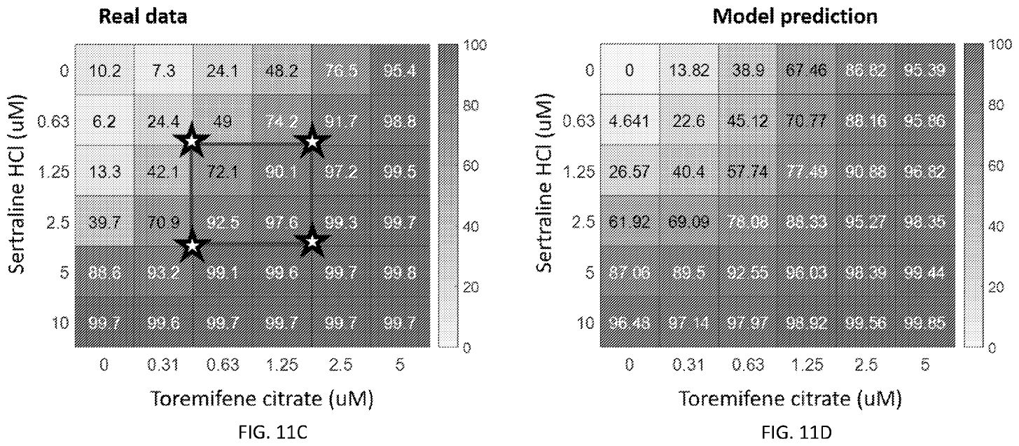

A pharmacodynamic (PD) model was employed to project the in vivo efficacy of drug combinations in humans. For combinations of (sertraline+bepridil) and (sertraline+toremifene), the in vitro IC₅₀s and hill coefficients for each drug, as well as the power factors which assess synergy (a), are provided in Table 14, below. The high R² (>0.93), indicates good model fit to the data. Hill coefficients exceeding 1 denote a steep dose response relationship and a˜1 indicates limited synergy on average across all combinations of drug levels. The good fit provided by the Bliss model implies potent multiplicative drug effects. As the model does not capture the regions of high synergy noted in reference (also see FIGS. 11A and 11C), its projection of in vivo potency at certain drug level combinations represents a minimum projection of potency.

TABLE 14
In vitro PD parameters for drug combinations.

| | Sertraline-Bepridil | | Sertraline-Toremifene | |
|---|---|---|---|---|
| | Drug 1 | Drug 2 | Drug 1 | Drug 2 |
| Parameters | Sertraline | Bepridil | Sertraline | Toremifene |
| mᵢ | 2.736 | 4.043 | 1.996 | 1.628 |
| IC_{50, i} | 2.961 | 2.688 | 1.888 | 0.7673 |

| | | |
|---|---|---|
| A | 1.005 | 1.013 |
| R² | 0.9435 | 0.9317 |

IC_{50, i} and mᵢ are the concentration of drug i (μM) required for 50% viral inhibition and the hill coefficient of drug i when drugs are applied in combination. a is a power factor, capturing the possible synergistic effects of drug combinations. R² is a statistical quantity indicating the proportion of variance explained by a model.

The empirical in vitro data from [22], displayed in FIGS. 11A and 11C, and model predictions for human in vivo efficacy under the condition where in vivo efficacy equals in vitro efficacy (see FIGS. 11B and 11D) were visually compared. For both combinations, the presently disclosed model provides good predictions of efficacies when both drugs are present, but somewhat poorer predictions when only one drug is present (compare top row and left-most column in FIGS. 11B and 11D vs. 11A and 11C). This does not affect results when investigating the effects of single drugs and drug combinations on EBOV viral load dynamics, because when only one drug is present, the efficacy of the single drug was stimulated using the PD model for the single drug (Equation (1)) and parameters provided in Table 7. Further, the model underestimated synergy in highly synergistic regions (indicated with boxes in FIGS. 11A and 11C), and hence projected efficacies are lower than empirical efficacies.

PK, PD and EBOV viral dynamic models (FIGS. 1A-1D) were combined to sertraline and bepridil alone and together to study if the drug combination provides more protection against EBOV infection than the components as single drug treatments. Treatments started on day 0, representing a post exposure prophylaxis scenario where people have had close contact with an infected patient, but do not yet have symptoms. With 10 days of 200 mg/day sertraline treatment, 10 days of 300 mg/day bepridil treatment, and 10 days of combined treatment (200 mg/day sertraline and 300 mg/day bepridil), the plasma concentration of sertraline and bepridil over time was projected. See FIGS. 12A-12C.

The percentage of viral replication inhibited over time under each treatment was stimulated assuming different in vivo IC₅₀s (see FIGS. 12D-12F), which were set at 0.1, 1, 5, and 10 times the in vitro IC₅₀s. This is because true in vivo IC₅₀s (the plasma concentration of drug required to inhibit replication by 50% in vivo) of a drug used alone or in combinations are unknown. Past studies on antiviral therapies demonstrated that the in vivo IC₅₀ could be 5-10 fold higher than in vitro estimates thereby necessitating higher drug peaks and troughs [39,40].

Sertraline alone limited only ˜30% of EBOV viral replication even when the in vivo IC₅₀ was assumed to be much lower (0.1×) than the in vitro IC₅₀ estimated from the dose response (D) of the single drug, with a ˜0.5-1.0 log consistent reduction in viral load. See FIG. 12G. Bepridil completely prevented viral replication and infection when the in vivo IC₅₀ was modeled to be 0.1× the in vitro IC₅₀. See FIGS. 12E and 12H. Moreover, when the in vivo IC₅₀ of bepridil was assumed to equal its in vitro estimate, it also suppressed viral load through day 14. With the drug combination (sertraline+bepridil), the efficacy is boosted such that anti-viral efficacy is seen even when the in vivo IC₅₀ was assumed to be 1× and 5× the in vitro IC₅₀ estimate from drug combination assays. See FIGS. 12F and 12I. Thus, it appears that the combination of sertraline and bepridil can provide better protection against EBOV infection compared with treatments with either sertraline alone or bepridil alone, when the human in vivo IC₅₀ is 1× or even 5× of the in vitro IC₅₀.

Similarly, it was investigated whether the combination of (sertraline+toremifene) can be more efficient in limiting EBOV viral replication than single drug therapy, by combining PK, PD and Ebola viral dynamic models. See FIGS. 13A-13I. Treatments started on day 0 and continued for 10 days. The concentration of sertraline and toremifene over time was projected in plasma or serum, respectively, with doses of 200 mg/day for sertraline (see FIG. 13A), 150 mg/day of toremifene with a 300 mg/day loading dose on day 0 (see FIG. 13B), and their combination (see FIG. 13C). The percentage of viral replication eliminated by each treatment was also stimulated assuming different values for in vivo IC₅₀s (see FIGS. 13D-13F) as described above for the combination (bepridil+sertraline). Sertraline as a single agent is discussed above with data from FIGS. 12A, 12D, and 12G replotted in FIGS. 13A, 13D, and 13G.

Toremifene alone was more efficient in limiting viral replication compared with sertraline alone when the in vivo IC₅₀ was assumed to be much lower (0.1×) than the in vitro IC₅₀ estimated in the dose response curve for the single drugs. See FIGS. 13E and 13H versus FIGS. 13D and 13G. However, neither sertraline alone nor toremifene alone protected against viral replication when the in vivo IC₅₀ was assumed to equal the in vitro values (i.e., for 1× in FIGS. 13D, 13E, 13G, and 13H). In contrast, the combination of (sertraline+toremifene) effectively increased the efficacy of the combination and eliminated viral replication with high potency when the in vivo IC₅₀ was assumed to be both 0.1× and 1× the in vitro IC₅₀. See FIGS. 13F and 13I. These findings suggest that (sertraline+toremifene) can provide better inhibition of viral replication than corresponding single drug therapies.

### SUMMARY

Filoviruses from the Ebolavirus and Marburgvirus genera cause life-threatening diseases of global concern. While an effective vaccine and two monoclonal antibody therapies have recently been approved for the management of disease caused by Ebola ebolavirus (EBOV), neither vaccines nor therapies are available for other consequential filoviruses including Sudan, Bundibugyo, and Tai Forest viruses, Marburgvirus, nor for potential emergent filoviruses [2-5]. There remains a pressing need for shelf-ready low-cost oral therapeutics that could be easily distributed and administered to mitigate disease at the earliest identification of a disease-causing filovirus, especially in resource-challenged regions around the world. A cost-effective treatment can ideally include a synergistic combination of orally available approved drugs. Synergistic combinations are favored over monotherapies due to their dose-lowering abilities and reduced chances of generating resistant viral strains [19,21,27,28]. Moreover, while two low molecular weight drugs appeared promising as intravenous monotherapies in non-human primate (NHP) models [62-64], two others provided by the oral route did not [48,65], and no small molecule monotherapies have yet proven highly effective against EBOV disease (EVD) in humans [66].

Towards the goal of developing a cocktail of orally available approved drugs with which to treat EVD patients, several laboratories have identified combinations of approved drugs that synergistically inhibit EBOV in cell cultures [22-26]. However, to date there has been no clear report of drug synergy against EBOV in an animal model. A common solubilizing vehicle for oral delivery of combinations of bepridil and sertraline and of sertraline and toremifene was identified herein and drug stability and efficacy upon storage of the combinations in this vehicle were demonstrated. A modeling study demonstrating that both drug pairs are projected to function synergistically in humans is also provided.

Bepridil, sertraline and toremifene have all shown survival benefits in the mouse model of EVD as single agents (Tables 10 and 11) (also see [6,7]), and the combinations of (bepridil+sertraline) and (sertraline+toremifene) were found to be synergistic against EBOV in Huh? cells (Table 8) (also see [22]). From previously reported data in Dyall 2018 [22], efficacies for the three drugs were enhanced ˜2 to 4-fold. In mice, the oral Cₘₐₓ/IC₅₀ for the drugs at the top doses proposed for initial testing, which are well-tolerated (see FIG. 10), were calculated to be 1.18, 0.56, and 1.74, respectively, for bepridil, sertraline and toremifene. See Table 13. Assuming ˜3-fold dose reductions when used in combinations all of the drugs should yield mouse plasma exposures greater than their respective IC₅₀ values (well above for bepridil and toremifene). Hence, it is expected that the proposed top oral doses can yield significant protection and that, perhaps requiring lower doses following a dose-down study, it will be possible to attain survival levels of 20-25%, thus enabling tests of whether either of these drug combinations functions synergistically in mice. All three drugs protect against multiple species of Ebolavirus and two species of Marburgvirus in cell cultures [6,7] and, in addition to strongly protecting mice against EBOV (see Tables 10 and 11) bepridil has provided strong protection in a mouse model of marburgvirus disease [47]. Hence, there is potential that the proposed combinations would be pan-filoviral.

Regarding sertraline, there is a prior study indicating that it did not protect macaques when given orally as a single agent [48]. In the cited study, the dose was 200 mg/day (in treat tablets), which represented a mean daily dose of 55 mg/kg. In the reported retrospective PK test of a single dose (50 mg/kg) of sertraline in uninfected macaques, the Cₘₐₓ was 179 ng/mL (0.58 NM) [48], which is ˜4.5-fold below the average IC₅₀ for sertraline against EBOV in liver cells in vitro. See Table 9. The in vivo IC₅₀ of sertraline (in infected macaques) can be even higher. The presently described simulations suggest a potent antiviral effect of the combination (bepridil+sertraline) even if the in vivo IC₅₀ in humans is 1× or 5× of the in vitro IC₅₀, with anti-viral activity seen even if the in vivo IC₅₀ is 10× the in vitro IC₅₀. See FIG. 12F. Collectively these findings suggest a need for a higher dose of sertraline to curb EBOV infections, and it is provided that the effectiveness of sertraline would be boosted in a combination, for example with bepridil. Similar reasoning supports a focus on drug combinations in drug repurposing efforts for diseases caused by other high consequence viral pathogens.

The present modeling studies (see FIGS. 12A-12I and 13A-13I) indicate that the combinations of (bepridil+sertraline) and (sertraline+toremifene) could deliver the additional potency required for a clinically meaningful decrease in EBOV viral load, depending on the actual in vivo IC₅₀. Indeed, in a retrospective analysis, viral loads in EVD survivors were found to be ˜1 log lower than in non-survivors [67,68], which is comparable to what the presently disclosed model projects for certain scenarios.

A dose down study of bepridil, sertraline and toremifene in V8 in mice challenged with EBOV can be performed, starting with the top doses proposed in Table 13. Once the ˜20-25% survival doses are determined, combined oral PK studies can be performed and it can be tested if the drug pairs (bepridil+sertraline) and (sertraline+toremifene), with each drug dosed to yield ˜25% survival, yield a greater than additive effect (i.e., greater than 50% survival). Following that, one or both of the pairs can be tested in an NHP model of EVD. This is currently advised, as drugs that have shown activity in rodent models as single agents have often failed in the NHP model [69]. Such an approach, when coupled with serial measurements of viral load can also provide initial estimates of the in vivo IC₅₀ in a mammal, which can then be leveraged to more accurately simulate clinical trials in humans, allowing more precise selection of combination agents, dose selection and dosing interval.

In recent years, other combinations [23,24,26] (see also Table 15, below) and other approved drugs have been identified with anti-EBOV activity in cell cultures; the latter include tilorone, pyronaridine and quinacrine [70], teicoplanin and arbidol [72]. Ones deemed suitable for oral delivery can warrant additional studies as drug combinations. In addition, if approved, several investigational drugs can warrant testing in combinations. These include novel selective estrogen receptor modulators [73] and amodiaquine analogues [74], agents that target the HR2 region of EBOV GP2 [75,76], potentially broad-spectrum drugs that target the viral polymerase [77], or drugs that target other EBOV or host cell proteins or their interactions [78-80]. Moreover, the possibility exists of adding a third drug as supported by current therapeutic strategies against HIV [19,20] and HCV [21]. In the case of EBOV, such combinations could substantially increase potency, particularly if the agents work via different mechanisms leading to multiplicative rather than additive antiviral effects. Furthermore, as many drugs, such as bepridil, sertraline and toremifene [6,7,47] show similar activity against multiple strains of Ebolavirus as well as against Marburgvirus, the potential exists to identify a drug combination that will inhibit most, if not all, filoviruses.

TABLE 15
Additional in vitro drug synergy tests.

| | | MacSynery | |
|---|---|---|---|
| Drug 1 | Drug 2 | (Av. LogV) | n |
| Aripiprazole | Piperacetazine | 15.0 | 2 ¹ |
| Aripiprazole | Amodiaquine | 3.2 | 3 |
| Aripiprazole | Bepridil | 7.5 | 2 |
| Favipiravir | Bepridil | 0.0 | 3 |
| Favipiravir | Amodiaquine | 7.6 | 3 |
| Favipiravir | Aripiprazole | 2.0 | 3 |
| Favipiravir | Ribavarin | 2.8 | 3 |
| Favipiravir | Sertraline | 0.9 | 2 |
| Favipiravir | Toremifene | 0.1 | 2 |
| Favipiravir | Azithromycin | 1.4 | 2 |
| Favipiravir | Clomiphene | 1.3 | 2 |
| Favipiravir | Favipiravir | 0.0 | 2 |

All synergy tests were performed in Huh7 cells with Ebov/Mak at moi 0.21 and analyzed using MacSynergy software as described in [22]. Data are presented at the 99.9% confidence level.
¹ In a third test using higher top doses of both drugs, the LogV was 71.95, which may be erroneous. Cytotoxicity was analyzed in parallel in three plates in all experiments. For the pairs showing strong (aripiprazole + piperacetazine) or moderate ((favipiravir + amodiaquine); (aripiprazole + bepridil)) synergy, toxicity was generally <5-20% throughout the plate, with higher toxicity seen in some wells containing high concentrations of drugs. Abbreviations: Av. LogV, average log volume; n, number of replicate experiments; each experiment performed in triplicate plates.

### Example 7

### Two-Way Modeling of PD of Drug Combinations

Methods:

Pharmacodynamic Data of Antiviral Drug Combinations:

PD data of drug combinations are reported in the format of dose response matrixes, where axes are drug concentrations, and each locus represents combined efficacy. Dose response matrixes of anti-Ebola drug combinations, including (sertraline+bepridil) and (sertraline+toremifene), were from reference [22]. Dose response matrixes of anti-SARS-CoV2 drug combinations, including (nitazoxanide+remdesivir) and (nitazoxanide+arbidol), were from reference [86].

Two-Way PD Modeling of Anti-Ebola and Anti-SARS-CoV-2 Drug Combinations:

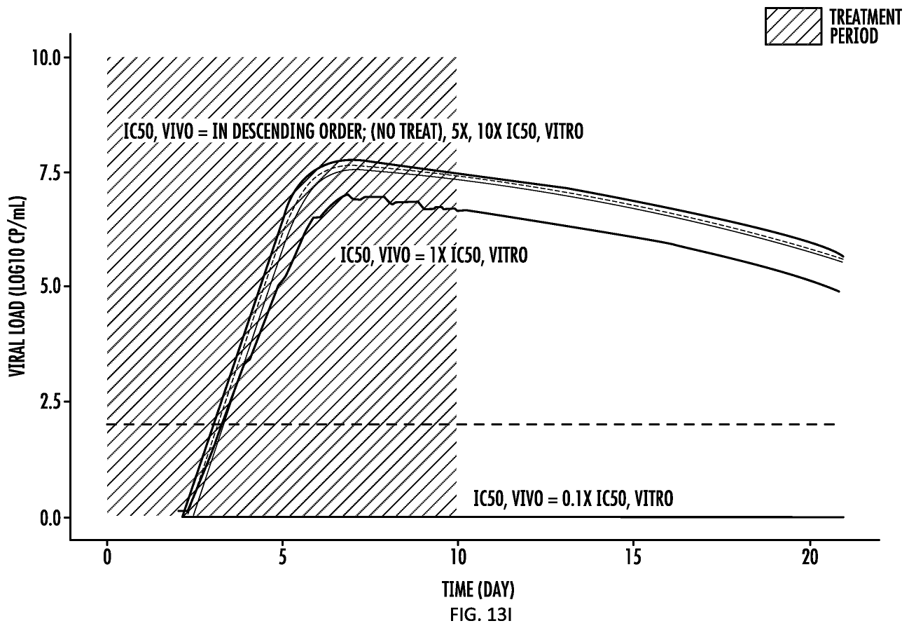

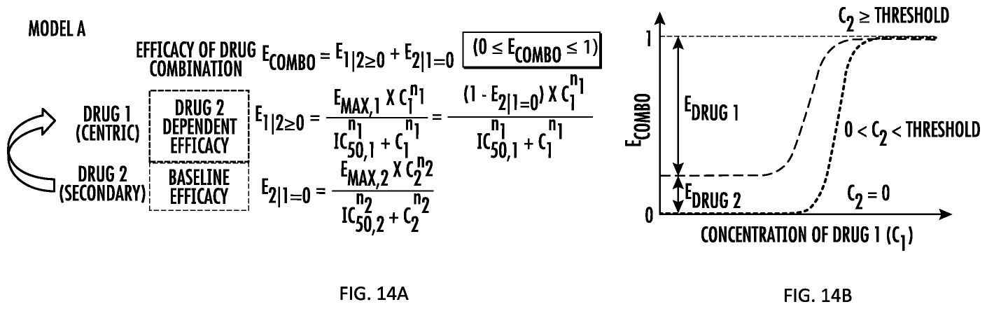

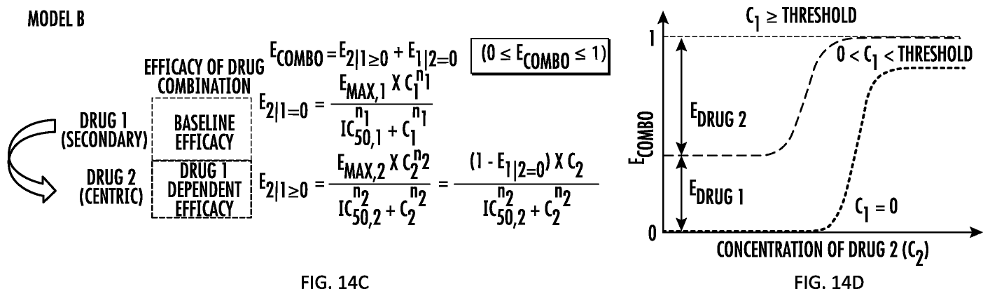

To model combined efficacy of anti-Ebola and anti-SARS-CoV-2 drug pairs over the dose-response matrix, a two-way pharmacodynamic model: equations (4-6), as described below, was used. The model assumes efficacy of a combination is the sum of the efficacy of a secondary drug and a centric drug affected by the secondary one. The model is flexible enough to switch roles of drugs in competing mechanistic models A and B. In Model A, drug 1 is assumed to be centric and drug 2 is assumed to be secondary. The combined efficacy equals the sum of efficacy provided by single drug 2 (E_{2|1=0}) and by drug 1 (E_{1|2≥0}) with drug 2 dependent PD parameters. See FIGS. 14A and 14C. In Model B, drug 2 is assumed to be centric and drug 1 is assumed to be secondary. The combined efficacy equals the sum of baseline efficacy of drug 1 (E_{1|2=0}) and efficacy provided by drug 2 (E_{2|1≥0}) with drug 1 dependent PD parameters. See FIGS. 14B and 14D.

For each model of a drug combination, baseline efficacy of the secondary drug was first quantified assuming monotherapy by fitting single efficacy data of the secondary drug to the standard dose-response curve (sigmoid Eₘₐₓ function). See equation (5), below. At each concentration of the secondary drug, the efficacy contributed by the centric drug was obtained by subtracting baseline efficacy from observed combined efficacy and further estimated Eₘₐₓ, IC₅₀ and hill coefficient of the centric drug by fitting to the sigmoid Eₘₐₓ function. See equation (6), below. Finally, combined efficacy of a pair was projected based on equation (4), below, and changes in combined efficacy compared to the sum of single drugs' efficacies:

ΔE=E_{combo}−min(100,(E_{1|2=0}+E_{2|1=0})).

The best PD model of combinations were determined by comparing the sum of squared error (SSE) and R² of Model A, Model B and the model described in Example 5 above:

$${E}_{c o m b o} = 100 - {( 100 \times {f}_{u 1} \times {f}_{u 2} )}^{a} ,$$

$$with {f}_{u i} = \frac{1}{1 + {( \frac{{D}_{i}}{{IC}_{50 , i}} )}^{{m}_{i}}} ,$$

where Dᵢ, IC_{50,i} and mᵢ represent the dose, concentration required for 50% efficacy, hill coefficient of drug i while a is the power factor aiming to capture synergistic effects in the dose-response matrix. Smaller SSE (higher R²) indicates better model prediction compared with empirical dataset.

Finally, an attempt was made to capture the relationship of secondary drug concentration on PD parameter values of the centric drug by using a non-mechanistic modeling approach. Specifically, for a PD parameter of a centric drug, several types of models (exponential, polynomial linear and sigmoidal) were fitted between concentration of the secondary drug and the PD parameter, to mathematically capture the relationship between the two without trying to understand the mechanism of drug-drug interaction.

### Discussion

Two-Way Pharmacodynamic Modeling for Efficacy of Drug Combinations

To capture concentration dependent drug-drug interactions and predict efficacy of drug combinations over the entire dose-response matrix, a two-way pharmacodynamic (TWPD) modeling approach was developed. In a combination composed of drug 1 and drug 2, either drug may be assigned as centric (subscript i) while the other is denoted as secondary (subscript j). The efficacy of a drug combination (E_{combo}) is the sum of the baseline efficacy contributed by the secondary (E_{j|i=0}) and the centric drug (E_{i|j≥0}):

E_{combo}=E_{i|j≥0}+E_{j|i=0} (4)

where,

$${E}_{j \vert i = 0} = \frac{{E}_{max , j} \times {C}_{j}^{{n}_{j}}}{{IC}_{50 , j}^{{n}_{j}} + {C}_{j}^{{n}_{j}}} \qquad ( 5 )$$

$${E}_{i \vert j \geq 0} = \frac{{E}_{max , i} \times {C}_{i}^{{n}_{i}}}{{IC}_{50 , i}^{{n}_{i}} + {c}_{i}^{{n}_{i}}} = \frac{( 1 - {E}_{j \vert i = 0} ) \times {C}_{i}^{{n}_{i}}}{{IC}_{50 , i}^{{n}_{i}} + {c}_{i}^{{n}_{i}}} \qquad ( 6 )$$

Cᵢ and Cⱼ represent concentration of the centric and secondary drug respectively. The secondary drug affects pharmacodynamic (PD) parameters of the centric, including maximum efficacy (E_{max,i}), drug concentration associated with 50% inhibition of infection (IC_{50,i}) and dose response slope (hill coefficient, nᵢ). The secondary drug dependent PD parameters of the centric drug reflect influences of the secondary drug on the dose response curve of the centric [108].

Calling the model above Model A, roles of drugs from centric to secondary can be switched in Model B and the two competing mechanistic hypotheses can be assessed. Thus, the TWPD provides information about efficacy of the drug combination, and informs which drug is more strongly influencing efficacy of the other (drug 1→drug 2 or drug 1←drug 2), or “directionality of synergy.” See FIGS. 14A-14D.

### Example 8

### Two-Way PD Modeling of Anti-Ebola Drug Combinations

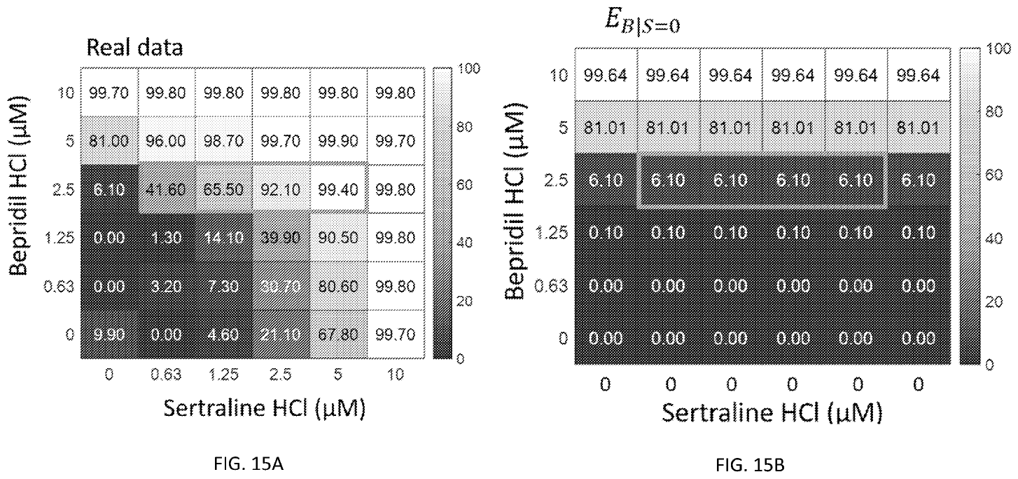

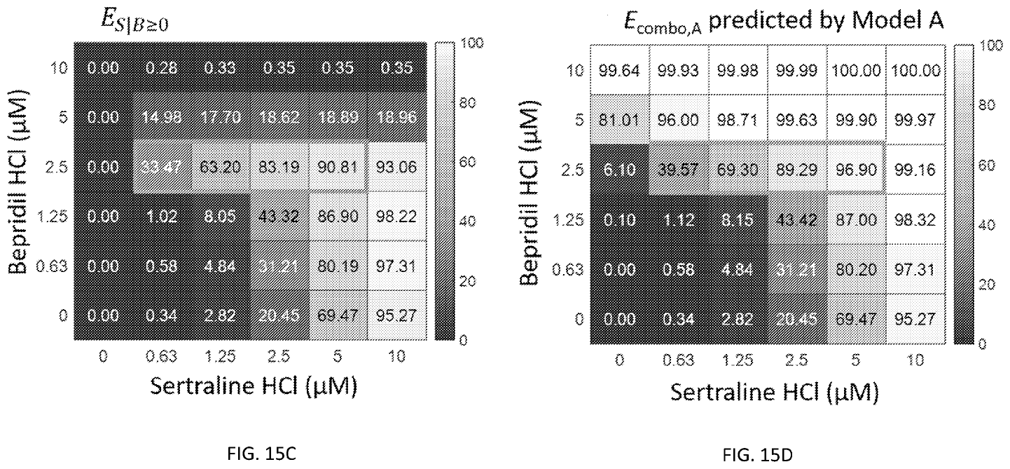

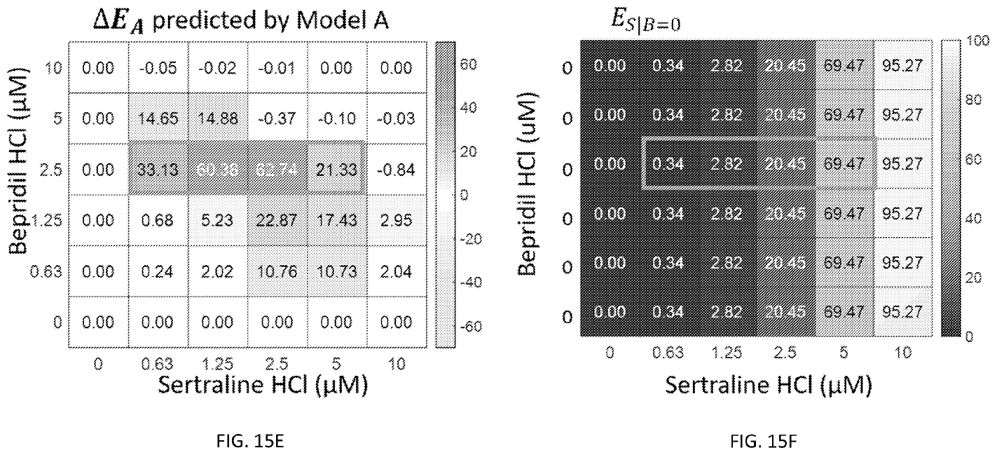

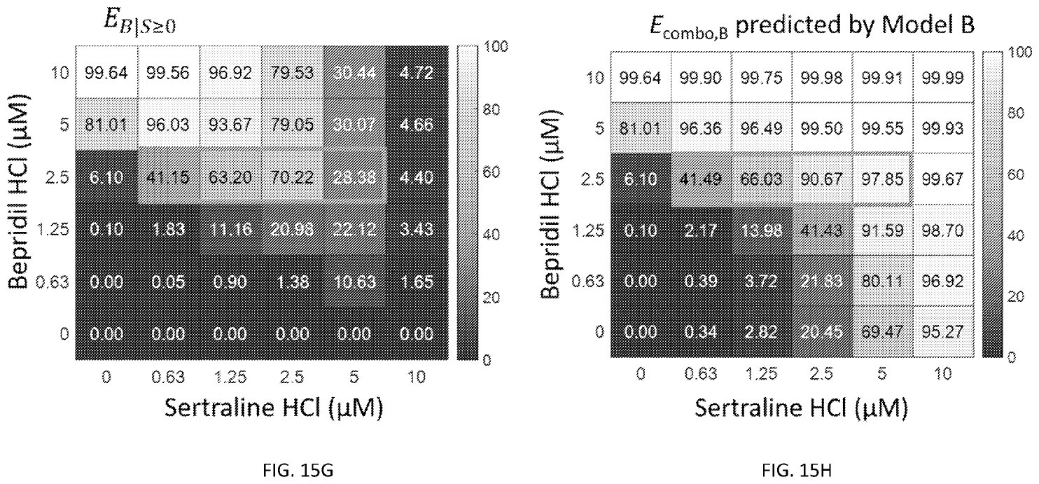

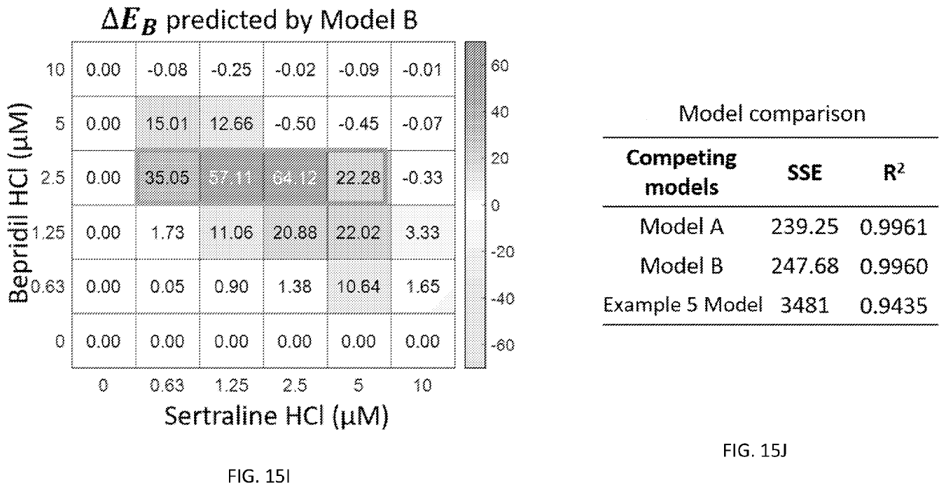

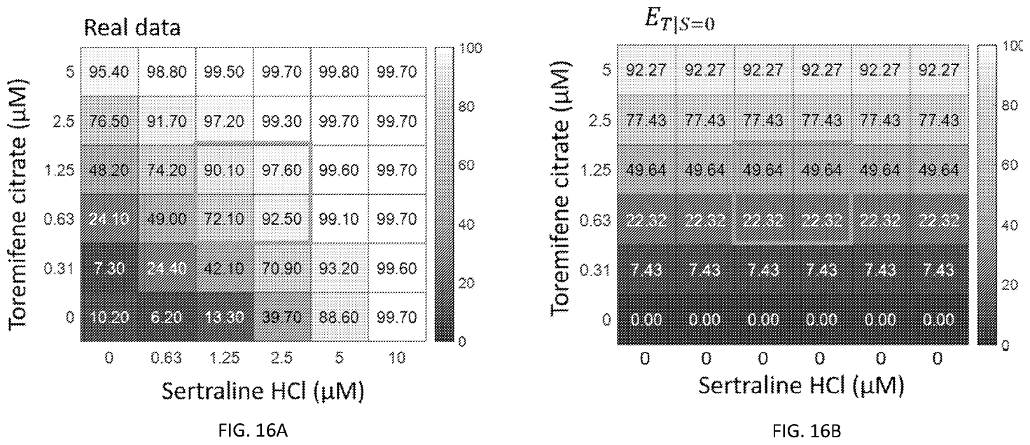

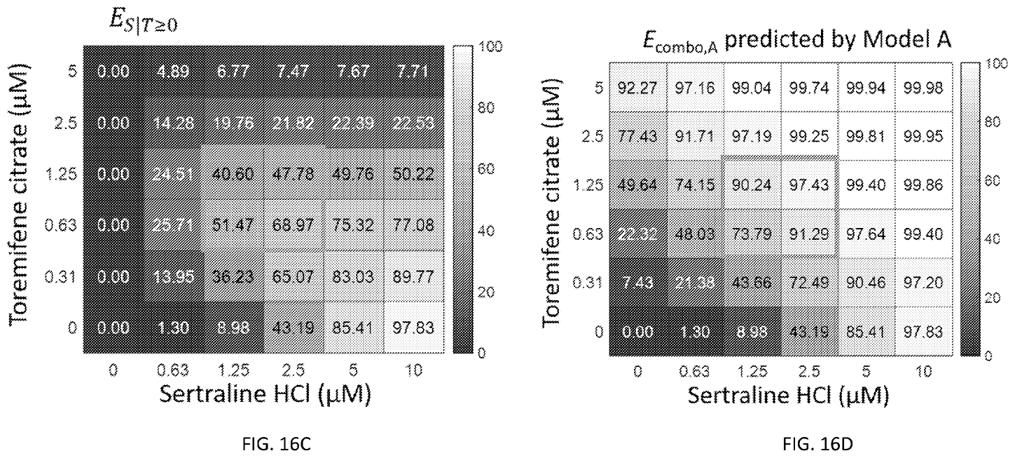

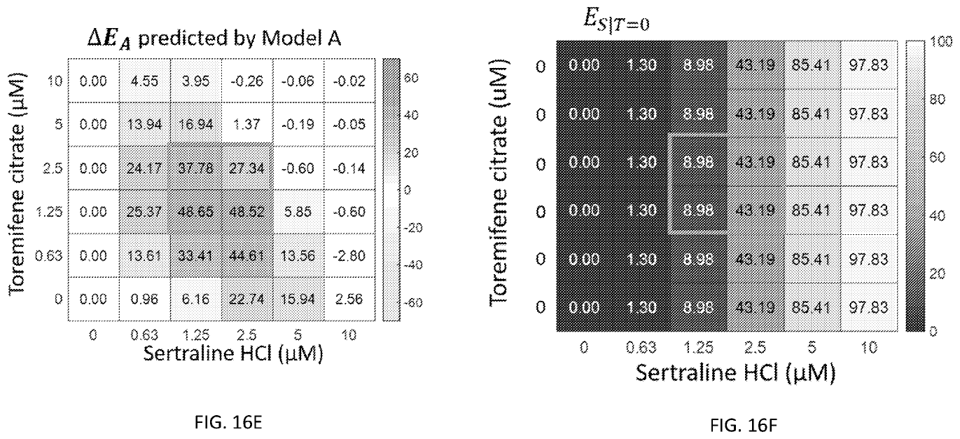

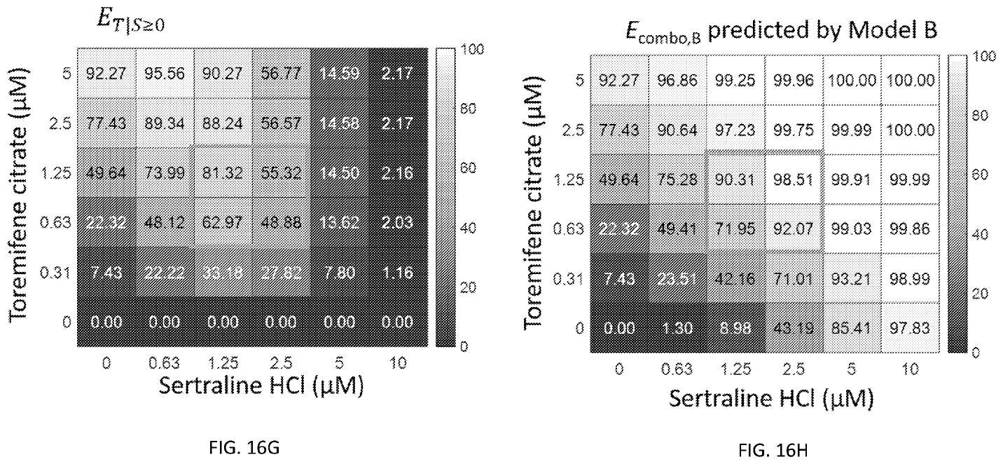

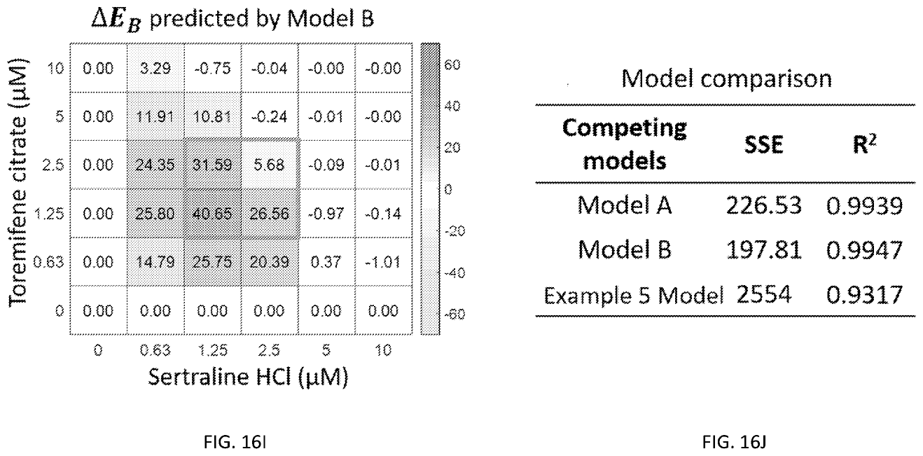

Empirically, the combination of sertraline+bepridil showed high synergy at 0.63 μM˜5 μM sertraline and 2.5 μM of bepridil (see FIG. 15A) [22], while sertraline+toremifene are highly synergistic at 1.25 μM˜2.5 μM sertraline and 0.63 μM˜1.25 μM toremifene (see FIG. 16A) [22].

By using the TWPD model, the dose response matrix data of (sertraline+bepridil) and (sertraline+toremifene) were fitted to Model A assuming sertraline as centric, and bepridil and toremifene as secondary, respectively, each of which provides increasing efficacy with ascending concentrations absent sertraline. See FIGS. 15B and 16B. Under this model, bepridil has a bi-phasic effect on sertraline: (i) 0 to 2.5 μM bepridil increases efficacy of sertraline, and (ii) levels greater than 2.5 μM bepridil decreases efficacy because the single drug effect of bepridil exceeds 80% at 5 μM leaving less room for added sertraline efficacy. See FIG. 15C. Toremifene has a similar bi-phasic effect on low levels of sertraline (≤2.5 μM) but monotonically reduces efficacy of higher concentrations of sertraline (≥5 μM). See FIG. 16C.

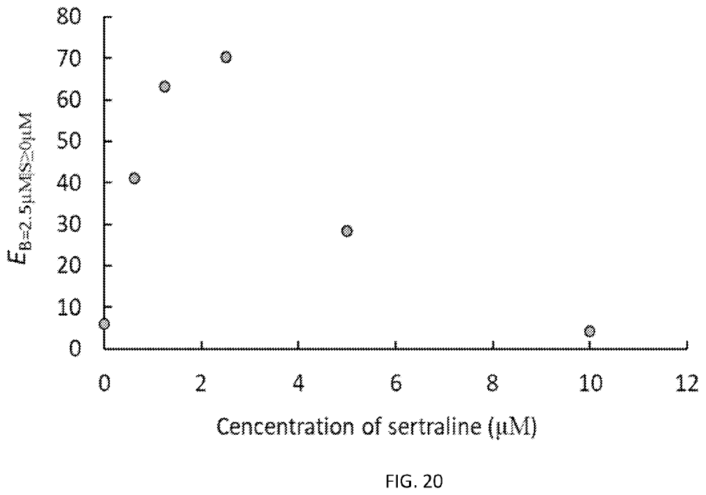

Model B of the anti-Ebola drug pairs were fitted assuming bepridil and toremifene are centric respectively, with sertraline as secondary contributing a baseline efficacy. See FIGS. 15F and 16F. Sertraline has bi-phasic effects on low to medium levels of bepridil (≤5 μM) (see FIG. 15G and FIG. 20) but across all concentrations of toremifene. See FIG. 16G.

For both anti-Ebola drug combinations, Model A and Model B projected high combined efficacies at high concentrations of centric and/or secondary drug (see FIGS. 15D, 15H, 16D, and 16H) as observed empirically. See FIGS. 15A and 16A. The accuracies of both models exceed that of the PD model described in Example 5 (see FIGS. 15J and 16J), indicating TWPD closely captures dynamics in dose response matrixes but did not, in this instance, provide inference with regard to the directionality of synergy. Moreover, changes in E combo demonstrate maximal increases in combined efficacies in highly synergistic regions with minimal increases at high concentrations of either agent which have close to 100% efficacy, limiting any extra possible effect of synergy. See FIGS. 15E, 15I, 16E, and 16I.

### Example 9

### Two-Way PD Modeling of Anti-SARS-CoV-2 Drug Combinations

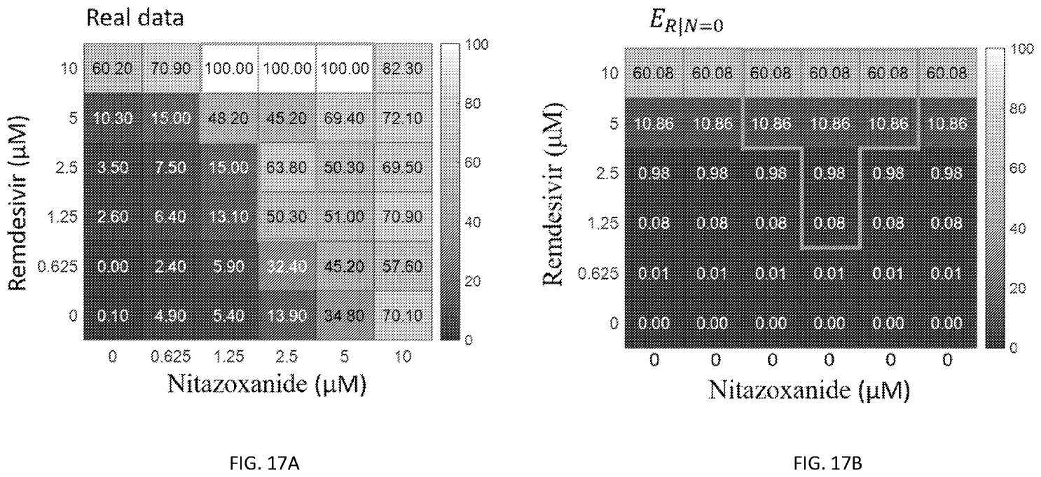

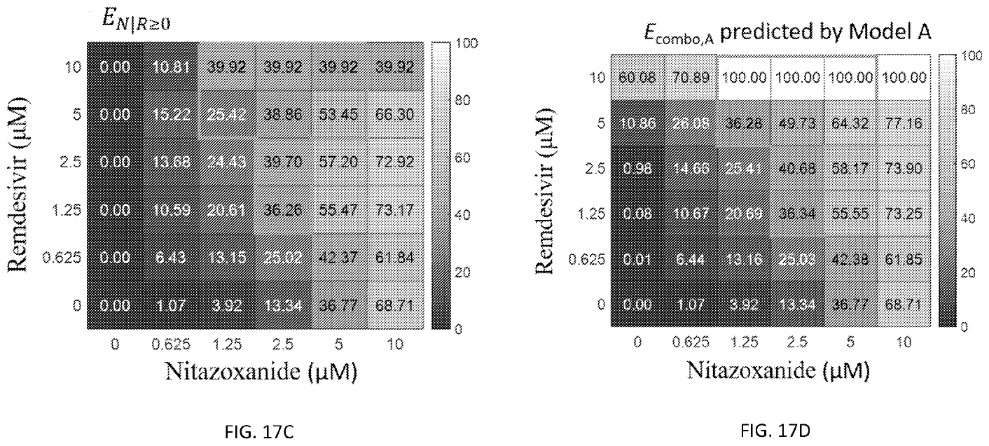

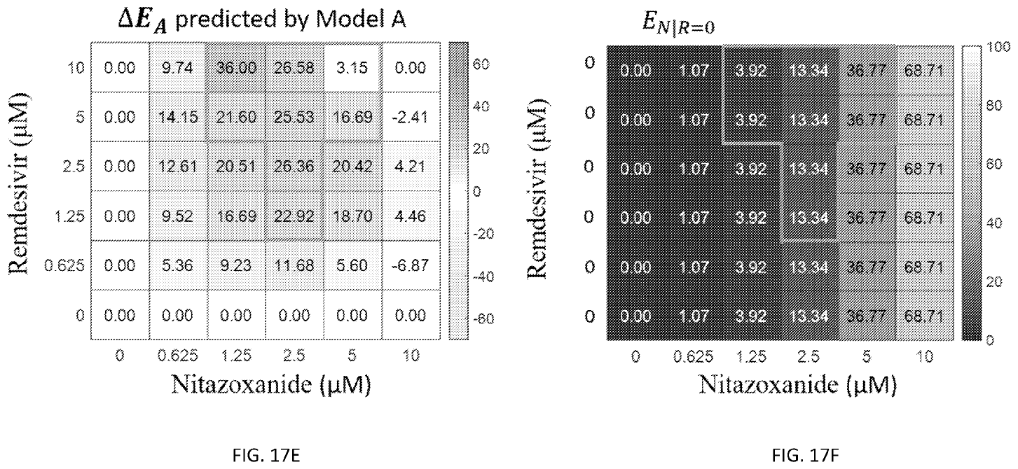

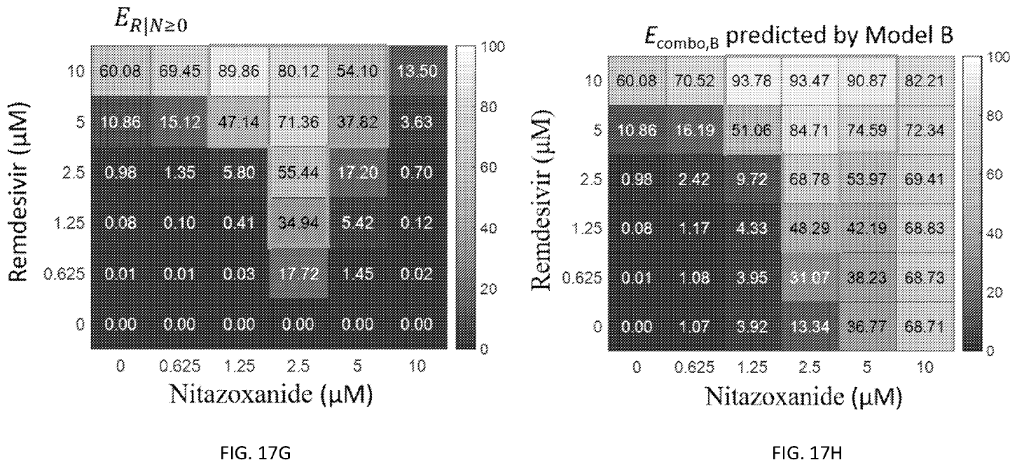

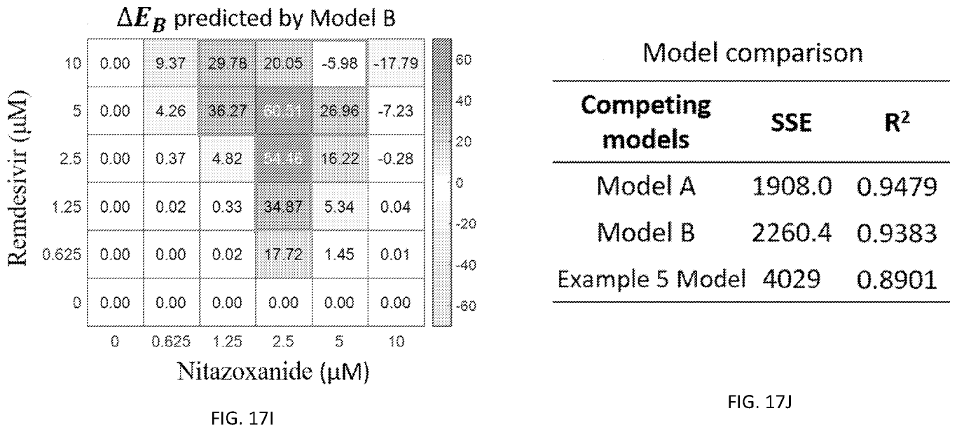

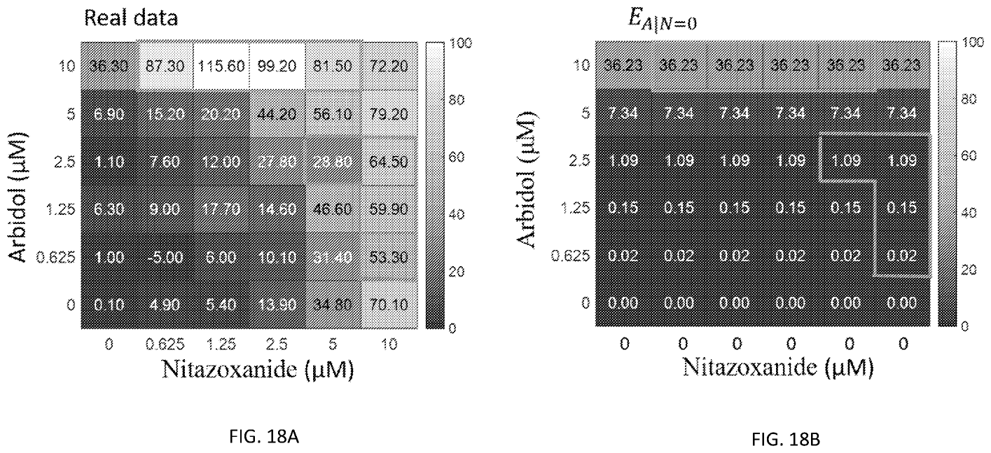

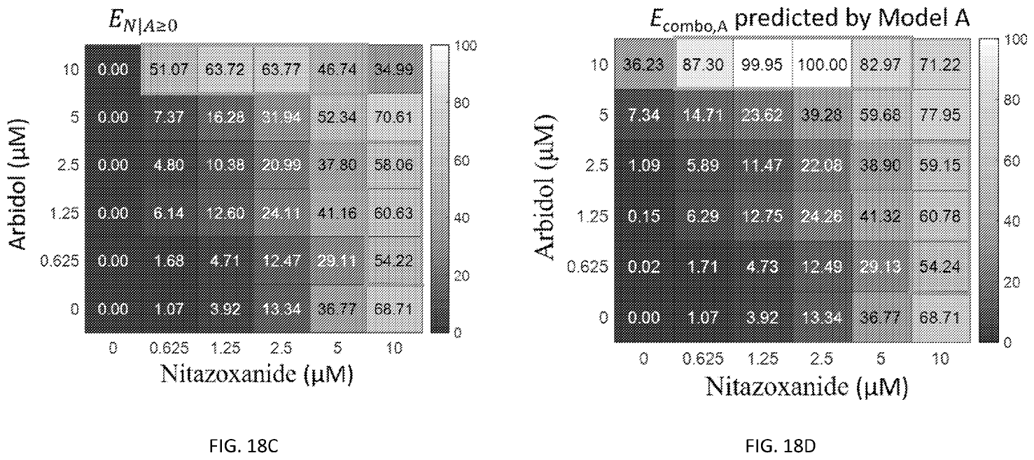

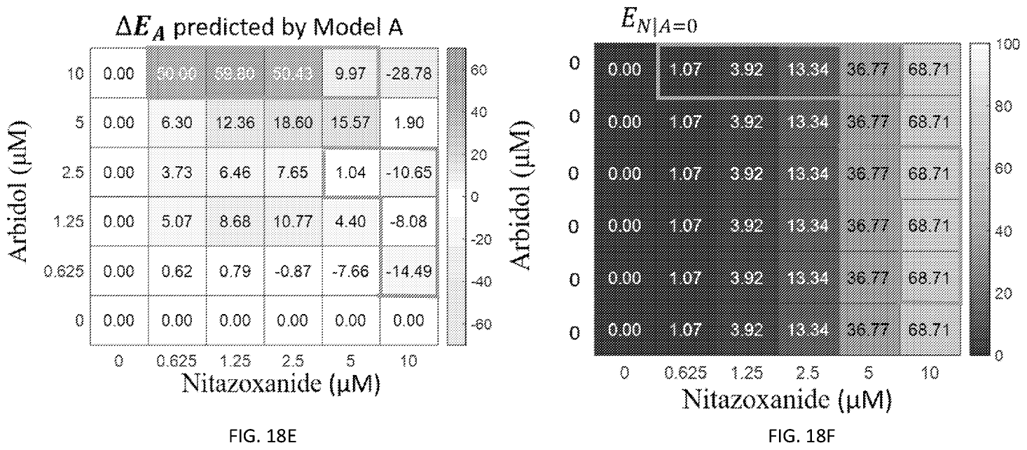

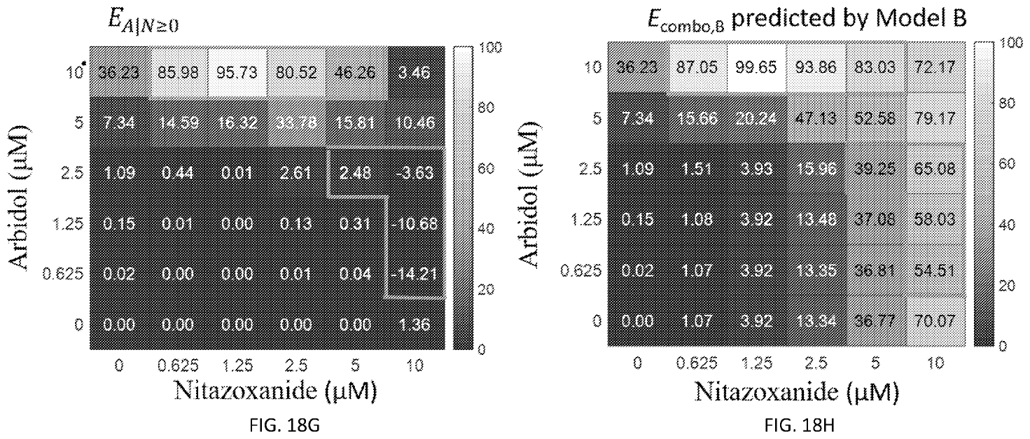

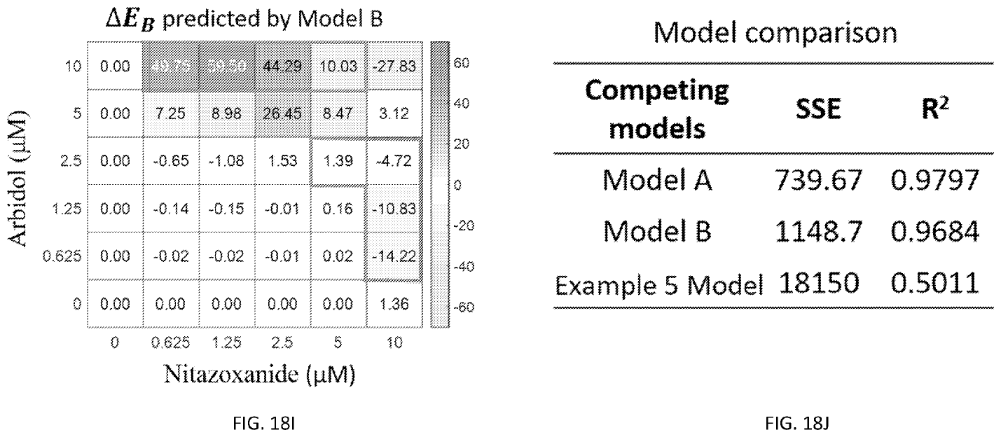

Arbidol and remdesivir have medium inhibition on SARS-CoV-2 in vitro when used alone [86]. See FIGS. 17A and 18A. The addition of nitazoxanide increases combined efficacy of (nitazoxanide+remdesivir) and (nitazoxanide+arbidol) in highly synergistic regions [86]. See FIGS. 17A and 18A.

The combined efficacy of each anti-SARS-CoV-2 drug combination was modeled to alternative models using TWPD. For Model A of (nitazoxanide+remdesivir) and (nitazoxanide+arbidol), nitazoxanide is centric, and secondary drugs are remdesivir and arbidol, respectively. The baseline efficacy provided by either remdesivir or arbidol only reaches 60% and 36% efficacy at 10 μM. See FIGS. 17B and 18B. Remdesivir consistently increases the efficacy of medium levels of nitazoxanide but has bi-phasic effects on low or high concentrations of the centric drug. See FIG. 17C. Arbidol at 10 μM is beneficial for a larger range of nitazoxanide (0.625˜5 μM) but is antagonistic to 10 μM nitazoxanide. See FIG. 18C.

For Model B of (nitazoxanide+remdesivir) and (nitazoxanide+arbidol), remdesivir and arbidol are centric in each combination, respectively, with nitazoxanide as secondary providing an increasing baseline efficacy. See FIGS. 17F and 18F. Nitazoxanide has bi-phasic effects across all levels of remdesivir when it crosses 2.5 μM nitazoxanide (see FIG. 17G), while only for medium to high concentrations of arbidol when it crosses 1.25 μM nitazoxanide. See FIG. 18G. For low concentrations of arbidol, 10 μM nitazoxanide reduces arbidol's efficacy. See FIG. 18G.

For both anti-SARS-CoV-2 drug combinations, low to moderate efficacies are ubiquitous in the dose-response matrixes, as projected by both Model A and Model B (see FIGS. 17D, 17H, 18D, and 18H), which recapitulate the empirical data (See FIGS. 17A and 18A) with better accuracies than the PD model described in Example 5. See FIGS. 17J and 18J. The similarity in performance of Model A and Model B does not provide for identification of directionality of synergy. Further, both models projected great improvements in combined efficacy in highly synergistic areas but diminished effect at high (10 μM) nitazoxanide concentration/antagonistic regions. See FIGS. 17E, 17I, 18E, and 18I.

### Example 10

### Effects of a Secondary Drug on PD Parameters of the Centric Drug

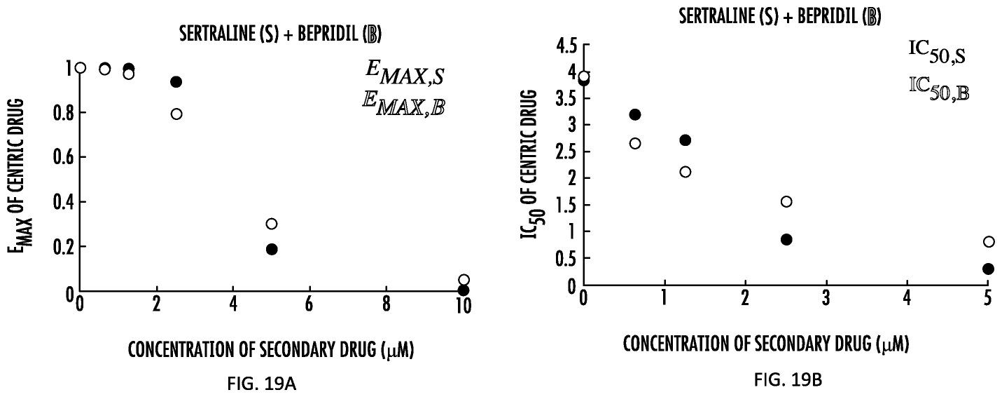

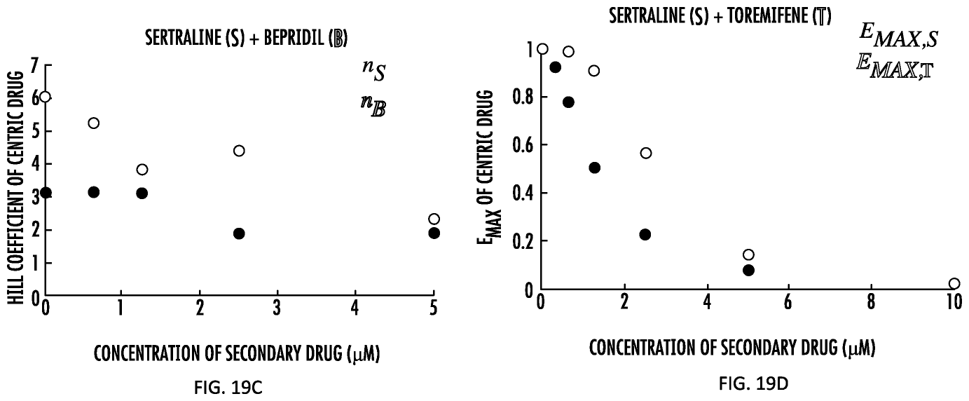

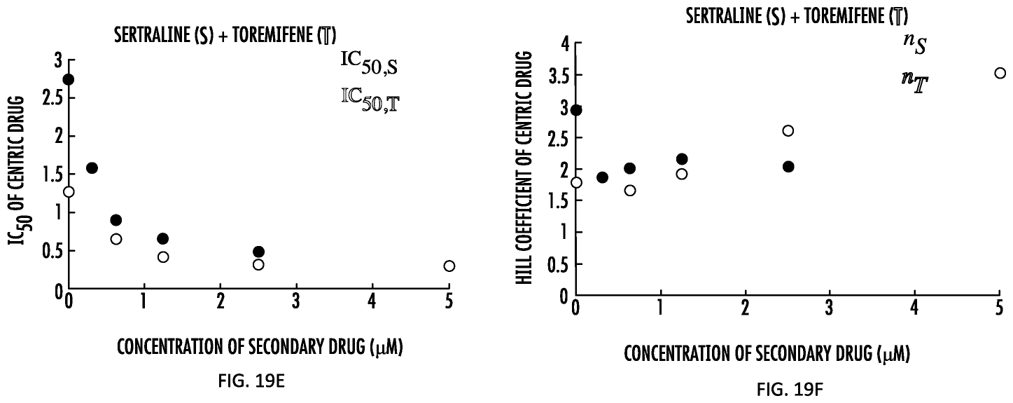

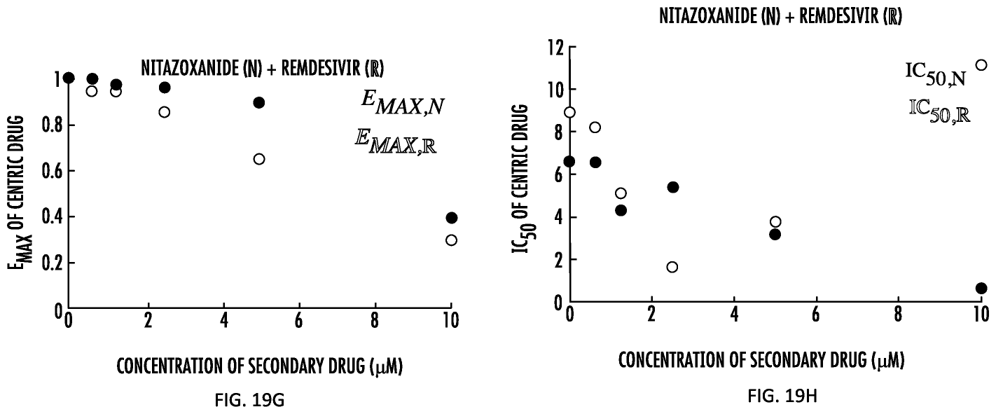

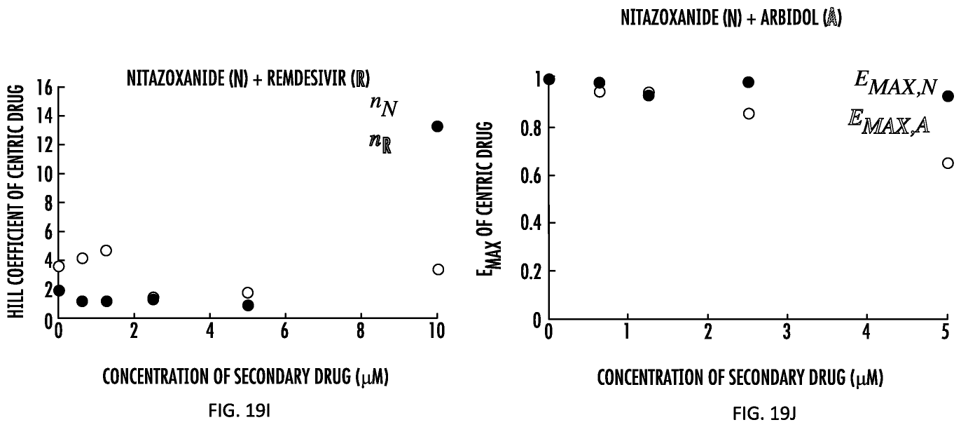

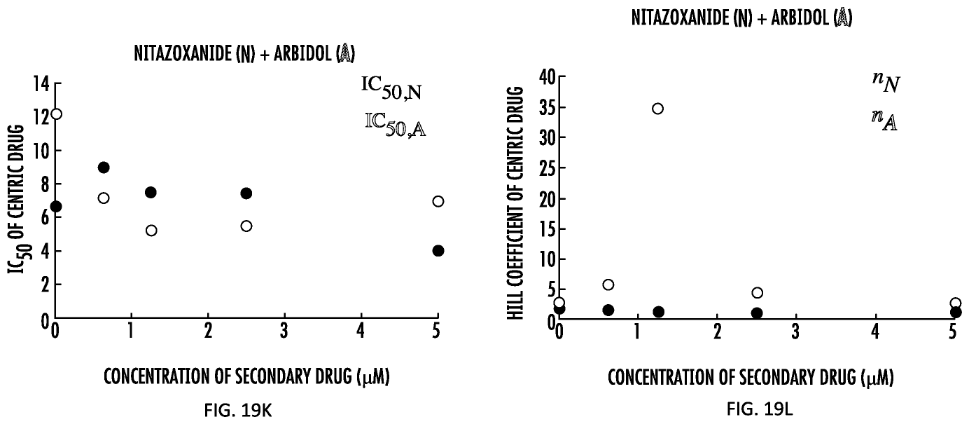

By applying TWPD to each drug combination, potential effects of a secondary drug on PD parameters of its centric one were identified. In Model A of (sertraline+bepridil) and (sertraline+toremifene) for Ebola virus, bepridil decreases Eₘₐₓ, IC₅₀ and hill coefficient of sertraline, with a pattern best captured by an inverted sigmoidal function. See FIGS. 19A-19C. Toremifene reduces the Eₘₐₓ and IC₅₀ of sertraline with a pattern best captured by an inverted sigmoidal function and an exponential decay curve respectively, but only lowers the hill coefficient of sertraline at low concentrations. See FIGS. 19D-19F. Areas of high potency occur within ranges of drug 2 concentrations (2-4 μM) where Eₘₐₓ remains high while IC₅₀ is relatively lowered.

In Model B of anti-Ebola drug combinations, sertraline is considered secondary while bepridil or toremifene is centric. Sertraline decreases Eₘₐₓ and IC₅₀ of bepridil and toremifene, following the pattern of an inverted sigmoidal function for Eₘₐₓ and exponential decay for IC₅₀. See FIGS. 19A, 19B, 19D, and 19E. However, sertraline affects the hill coefficient of bepridil and toremifene in opposite directions with decreases and increases respectively. See FIGS. 19C and 19F.

As with anti-Ebola drug combinations, impacts of secondary drug on the centric drug were observed for anti-SARS-CoV-2 drug combinations, though the secondary drugs in anti-SARS-CoV-2 drug pairs are not as potent alone as those in anti-Ebola drug combinations. As a result, Eₘₐₓ of the centric drugs demonstrate less decline for both (nitazoxanide+remdesivir) and (nitazoxanide+arbidol) irrespective of whether Model A or Model B is applied. See FIGS. 19G and 19J. In this sense, the synergistic combinatorial effects are more impactful for SARS-CoV-2 than Ebola.

In Model A of (nitazoxanide+remdesivir) and (nitazoxanide+arbidol), the IC₅₀ of nitazoxanide decays exponentially with remdesivir (see FIG. 19H), while it is negatively influenced by arbidol. See FIG. 19K. Remdesivir and arbidol have similar effects on the hill coefficient of nitazoxanide, patterns of which best fit to bi-exponential functions. See FIGS. 191 and 19L. The hill coefficient of nitazoxanide at 10 μM remdesivir has a relatively high estimated value. Without being bound to any one theory, this result could be attributed to identifiability issues associated with steep dose response curves.

In Model B, nitazoxanide, the secondary drug, has nonmonotonic effects on remdesivir and arbidol. The IC₅₀ of remdesivir decreases linearly up to 2.5 μM nitazoxanide and then increases linearly to capture antagonism at high concentrations. See FIG. 19H. Nitazoxanide changes the IC₅₀ of arbidol, the curve of which has a bi-exponential shape with a deep decline at low concentrations of nitazoxanide but a slight increase afterwards. See FIG. 19K. The hill coefficient of remdesivir also changes in a nonmonotonic manner with increases and decreases in different ranges of nitazoxanide (See FIG. 19I), while the hill coefficient of arbidol increases exponentially, reaching a peak at 1.25 μM nitazoxanide, and then drops exponentially. See FIG. 19L.

### Example 11

### Discussion of Examples 7-10

Non-linear concentration dependent drug-drug interactions are observed in high throughput screenings of synergistic drug combinations. Recapitulating these values with pharmacodynamic modeling is challenging and can limit simulations of treatment trials. A two-way pharmacodynamic modeling (TWPD) approach, considering the role of a drug in a combination being centric or secondary and vice versa, is described herein. The presently disclosed TWPD model captures concentration dependent drug-drug interactions over the entire dose-response matrix and was applied to four antiviral binary drug combinations with great accuracy. For the four pairs, the predictive power of TWPD is direction independent, meaning the role of a drug does not influence model performance. The presently disclosed TWPD model is an efficient modeling approach to predict pharmacodynamics of drug combinations with concentration dependent interactions and can be used to explore drug pair effects when combined with pharmacokinetic and intra-host viral dynamic models.

The presently disclosed TWPD modeling approach contrasts with the Zero interaction potency (ZIP) [108] and general pharmacodynamic interaction (GPDI) models [109]. All three models consider shifts in PD parameters of a drug caused by an additional drug if they interact. The ZIP model focuses on diagnosing synergy while GPDI and TWPD models capture concentration dependent drug-drug interactions. However, the presently disclosed TWPD model is more general by making no assumption on which PD parameters can be altered by adding a secondary drug, rather than assuming changes in potency as with ZIP or changes in IC₅₀ for competitive interaction and Eₘₐₓ for allosteric interaction as with GPDI. As described herein, using the presently disclosed TWPD approach, it was found that a secondary drug can have complicated impacts on all three parameters in the sigmoid function, including Eₘₐₓ, IC₅₀ (potency) and hill coefficient, putting the maximum effect model assumption estimated in the GPDI model at risk. In contrast, the presently disclosed TWPD approach bypasses the process of estimating interaction terms based on PD parameters. Thus, TWPD modeling is more flexible and requires less prior knowledge about drug-drug interactions.

Drug combination screening is mostly conducted for drug pairs. The presently disclosed TWPD could also be applied to triple drug combinations by following the logic that a third drug adds additional effects on PD parameters of the centric drug if it interacts with the other two drugs. The concentration dependent impacts of a secondary and tertiary drug on the PD parameters of the centric drug could be quantified successively. The directionality of the model could then be examined by switching roles of drugs in alternative models.

For the pairs modeled in the examples herein, anti-SARS-CoV-2 combinations (nitazoxanide+remdesivir) and (nitazoxanide+arbidol) demonstrated more benefit than anti-Ebola drug combinations (sertraline+bepridil) and (sertraline+toremifene) when drug concentrations are relatively high. Single drug components in the anti-Ebola drug combinations achieve high efficacy when used as single drugs, which can be therapeutically sufficient to inhibit viruses. Drug synergy is more prominent at lower concentrations of anti-Ebola drug pairs than high concentrations. Single drug components for anti-SARS-CoV-2 drug pairs only have low or moderate efficacy at 10 μM with limited viral inhibition. Combining them with a synergistic agent can provide for relatively large improvements. In Vero 6 cells, the combined efficacy of anti-SARS-CoV-2 drug combinations in the highly synergistic areas on the dose-response matrix increases considerably compared with the sum of single drugs' efficacies and achieves close to 100% combined efficacy.

Peak and trough concentrations of each component drug in the anti-Ebola and anti-SARS-CoV drug combinations make it plausible that synergy can augment potency for a portion of the dosing interval. Peak and trough concentrations of bepridil are 3.63 (±1.48) μM and 2.60 (±1.21) μM [120]. Sertraline has higher average peak and trough concentrations in women (Cₘₐₓ=0.54 (±0.21) μM and Cₘᵢₙ=0.35 (±0.19) μM) than men (Cₘₐₓ=0.39 (±0.07) μM and Cₘᵢₙ=0.21 (±0.08) μM) [83]. The maximal concentration of toremifene is 1.18 (±0.42) μM with a trough concentration of 0.29 μM [36]. Based on peak and trough concentrations of anti-Ebola drug components, bepridil alone is more effective than sertraline and toremifene with an estimated maximal efficacy around 81% at Cₘₐₓ.

For the anti-SARS-CoV-2 drug combinations, peak and trough concentrations are 0.77 (±0.23) μM and 0.32 (±0.09) μM for arbidol [121], and 17.34 (±13.61) and 3.02 (±7.43) for tizoxanide (a metabolite of nitazoxanide) [122], respectively. Remdesivir reaches peak concentration of 5.34 (±0.03) μM at 1.03 (±0.01) hour and has a short half-life (median t_{1/2}≈1 hour) without accumulation [123]. The combination of (nitazoxanide+remdesivir) is therefore more likely to increase combined drug efficacy compared with (nitazoxanide+arbidol) because plasma concentrations of arbidol are low with dosing of 200 mg every 8 hours.

In addition to pharmacokinetics, the effects of a secondary drug on PD parameters of its centric drug can also be considered when prioritizing drug pairs from high throughput screenings. Prioritizing drug pairs for further investigation can favor scenarios where secondary drugs lower the IC₅₀ of a centric drug while maintaining its Eₘₐₓ. Increases in hill coefficient of the centric drug is also beneficial because efficacy of the centric drug will rise more quickly over a narrow range of drug concentrations.

To fully realize the benefits of synergistic repurposed drug combinations, regimens need to be optimized such that the pharmacokinetics of a pair provide the largest proportion of time within the dosing interval at high synergy. Mathematical modeling can be an efficient way to explore possible dosing combinations by combining PD of drug pairs with pharmacokinetic models, which reflect fluctuations of drug concentrations in humans, as well as intra-host viral dynamic models. The presently disclosed TWPD approach, which accurately estimates combined efficacy across the dose-response matrix, can help to provide precise predictions on therapeutic effects of drug combinations and optimal dosing regimens, thereby facilitating effective countermeasures against (re)emerging viruses of pandemic potential.

### REFERENCES

All references listed in the instant disclosure, including but not limited to all patents, patent applications and publications thereof, scientific journal articles, and database entries are incorporated herein by reference in their entireties to the extent that they supplement, explain, provide a background for, and/or teach methodology, techniques, and/or compositions employed herein. The discussion of the references is intended merely to summarize the assertions made by their authors. No admission is made that any reference (or a portion of any reference) is relevant prior art. Applicants reserve the right to challenge the accuracy and pertinence of any cited reference.
1. Kuhn, J. H.; Amarasinghe, G. K.; Basler, C. F.; Bavari, S.; Bukreyev, A.; Chandran, K.; Crozier, I.; Dolnik, O.; Dye, J. M.; Formenty, P. B. H.; et al. Ictv Report Consortium ICTV virus taxonomy profile: Filoviridae. J. Gen. Viral. 2019, 100, 911-912.
2. Feldmann, H.; Sprecher, A.; Geisbert, T. W. Ebola. N. Engl. J. Med. 2020, 382, 1832-1842.
3. Malvy, D.; McElroy, A. K.; de Clerck, H.; Gunther, S.; van Griensven, J. Ebola virus disease. Lancet 2019, 393, 936-948.
4. Lo, T. Q.; Marston, B. J.; Dahl, B. A.; De Cock, K. M. Ebola: Anatomy of an epidemic. Annu. Rev. Med. 2017, 68, 359-370.
5. Yang, X.-L.; Tan, C. W.; Anderson, D. E.; Jiang, R.-D.; Li, B.; Zhang, W.; Zhu, Y.; Lim, X. F.; Zhou, P.; Liu, X.-L.; et al. Characterization of a filovirus (Mengla virus) from Rousettus bats in China. Nat. Microbiol. 2019, 4, 390-395.
6. Johansen, L. M.; Brannan, J. M.; Delos, S. E.; Shoemaker, C. J.; Stossel, A.; Lear, C.; Hoffstrom, B. G.; Dewald, L. E.; Schomberg, K. L.; Scully, C.; et al. FDA-approved selective estrogen receptor modulators inhibit Ebola virus infection. Sci. Transl. Med. 2013, 5, 190ra79.
7. Johansen, L. M.; DeWald, L. E.; Shoemaker, C. J.; Hoffstrom, B. G.; Lear-Rooney, C. M.; Stossel, A.; Nelson, E.; Delos, S. E.; Simmons, J. A.; Grenier, J. M.; et al. A screen of approved drugs and molecular probes identifies therapeutics with anti-Ebola virus activity. Sci. Transl. Med. 2015, 7, 290ra89.
8. Madrid, P. B.; Chopra, S.; Manger, I.D.; Gilfillan, L.; Keepers, T. R.; Shurtleff, A. C.; Green, C. E.; Iyer, L. V.; Dilks, H. H.; Davey, R. A.; et al. A systematic screen of FDA-approved drugs for inhibitors of biological threat agents. PLoS ONE 2013, 8, e60579.
9. Kouznetsova, J.; Sun, W.; Martinez-Romero, C.; Tawa, G.; Shinn, P.; Chen, C. Z.; Schimmer, A.; Sanderson, P.; McKew, J. C.; Zheng, W.; et al. Identification of 53 compounds that block Ebola virus-like particle entry via a repurposing screen of approved drugs. Emerg. Microbes Infect. 2014, 3, e84.
10. Dowall, S. D.; Bewley, K.; Watson, R. J.; Vasan, S. S.; Ghosh, C.; Konai, M. M.; Gausdal, G.; Lorens, J. B.; Long, J.; Barclay, W.; et al. Antiviral Screening of Multiple Compounds against Ebola Virus. Viruses 2016, 8.
11. Luthra, P.; Liang, J.; Pietzsch, C. A.; Khadka, S.; Edwards, M. R.; Wei, S.; Alexander, B.; Posner, B.; Bukreyev, A.; Ready, J. M.; et al. A high throughput screen identifies benzoquinoline compounds as inhibitors of Ebola virus replication. Antiviral Res. 2018, 150, 193-201.
12. Schafer, A.; Cheng, H.; Xiong, R.; Soloveva, V.; Retterer, C.; Mo, F.; Bavari, S.; Thatcher, G.; Rong, L. Repurposing potential of 1st generation H1-specific antihistamines as anti-filovirus therapeutics. Antiviral Res. 2018, 157, 47-56.
13. Lee, N.; Shum, D.; Konig, A.; Kim, H.; Heo, J.; Min, S.; Lee, J.; Ko, Y.; Choi, I.; Lee, H.; et al. High-throughput drug screening using the Ebola virus transcription- and replication-competent virus-like particle system. Antiviral Res. 2018, 158, 226-237.
14. Bixler, S. L.; Duplantier, A. J.; Bavari, S. Discovering drugs for the treatment of ebola virus. Curr. Treat. Options Infect. Dis. 2017, 9, 299-317.
15. Iversen, P. L.; Kane, C. D.; Zeng, X.; Panchal, R. G.; Warren, T. K.; Radoshitzky, S. R.; Kuhn, J. H.; Mudhasani, R. R.; Cooper, C. L.; Shurtleff, A. C.; et al. Recent successes in therapeutics for Ebola virus disease: No time for complacency. Lancet Infect. Dis. 2020, 20, e231-e237.
16. Edwards, M. R.; Basler, C. F. Current status of small molecule drug development for Ebola virus and other filoviruses. Curr. Opin. Viral. 2019, 35, 42-56.
17. Salata, C.; Calistri, A.; Alvisi, G.; Celestino, M.; Parolin, C.; Pahl, G. Ebola virus entry: From molecular characterization to drug discovery. Viruses 2019, 11, 274.
18. Bai, J. P. F.; Hsu, C.-W. Drug repurposing for ebola virus disease: Principles of consideration and the animal rule. J. Pharm. Sci. 2019, 108, 798-806.
19. Gulick, R. M.; Mellors, J. W.; Havlir, D.; Eron, J. J.; Gonzalez, C.; McMahon, D.; Richman, D. D.; Valentine, F. T.; Jonas, L.; Meibohm, A.; et al. Treatment with indinavir, zidovudine, and lamivudine in adults with human immunodeficiency virus infection and prior antiretroviral therapy. N. Engl. J. Med. 1997, 337, 734-739.
20. Jilek, B. L.; Zarr, M.; Sampah, M. E.; Rabi, S. A.; Bullen, C. K.; Lai, J.; Shen, L.; Siliciano, R. F. A quantitative basis for antiretroviral therapy for HIV-1 infection. Nat. Med. 2012, 18, 446-451.
21. Shafran, S. D.; Shaw, D.; Charafeddine, M.; Agarwal, K.; Foster, G. R.; Abunimeh, M.; Pilot-Matias, T.; Pothacamury, R. K.; Fu, B.; Cohen, E.; et al. Efficacy and safety results of patients with HCV genotype 2 or 3 infection treated with ombitasvir/paritaprevir/ritonavir and sofosbuvir with or without ribavirin (QUARTZ II-III). J. Viral. Hepat. 2018, 25, 118-125.
22. Dyall, J.; Nelson, E. A.; DeWald, L. E.; Guha, R.; Hart, B. J.; Zhou, H.; Postnikova, E.; Logue, J.; Vargas, W. M.; Gross, R.; et al. Identification of combinations of approved drugs with synergistic activity against ebola virus in cell cultures. J. Infect. Dis. 2018, 218, S672-S678.
23. Sun, W.; He, S.; Martinez-Romero, C.; Kouznetsova, J.; Tawa, G.; Xu, M.; Shinn, P.; Fisher, E.; Long, Y.; Motabar, O.; et al. Synergistic drug combination effectively blocks Ebola virus infection. Antivir. Res. 2017, 137, 165-172.
24. Bekerman, E.; Neveu, G.; Shulla, A.; Brannan, J.; Pu, S.-Y.; Wang, S.; Xiao, F.; Barouch-Bentov, R.; Bakken, R. R.; Mateo, R.; et al. Anticancer kinase inhibitors impair intracellular viral trafficking and exert broad-spectrum antiviral effects. J. Clin. Investig. 2017, 127, 1338-1352.
25. McCarthy, S. D. S.; Majchrzak-Kita, B.; Racine, T.; Kozlowski, H. N.; Baker, D. P.; Hoenen, T.; Kobinger, G. P.; Fish, E. N.; Branch, D. R. A Rapid Screening Assay Identifies Monotherapy with Interferon-B and Combination Therapies with Nucleoside Analogs as Effective Inhibitors of Ebola Virus. PLoS Negl. Trop. Dis. 2016, 10, e0004364.
26. Du, X.; Zuo, X.; Meng, F.; Wu, F.; Zhao, X.; Li, C.; Cheng, G.; Qin, F. X.-F. Combinatorial screening of a panel of FDA-approved drugs identifies several candidates with anti-Ebola activities. Biochem. Biophys. Res. Commun. 2020, 522, 862-868.
27. Lehar, J.; Krueger, A. S.; Avery, W.; Heilbut, A. M.; Johansen, L. M.; Price, E. R.; Rickles, R. J.; Short, G. F.; Staunton, J. E.; Jin, X.; et al. Synergistic drug combinations tend to improve therapeutically relevant selectivity. Nat. Biotechnol. 2009, 27, 659-666.
28. Cheng, Y.-S.; Williamson, P. R.; Zheng, W. Improving therapy of severe infections through drug repurposing of synergistic combinations. Curr. Opin. Pharmacol. 2019, 48, 92-98.
29. Madelain, V.; Baize, S.; Jacquot, F.; Reynard, S.; Fizet, A.; Barron, S.; Solas, C.; Lacarelle, B.; Carbonnelle, C.; Mentre, F.; et al. Ebola viral dynamics in nonhuman primates provides insights into virus immuno-pathogenesis and antiviral strategies. Nat. Commun. 2018, 9, 4013.
30. Coves-Datson, E. M.; Dyall, J.; DeWald, L. E.; King, S. R.; Dube, D.; Legendre, M.; Nelson, E.; Drews, K. C.; Gross, R.; Gerhardt, D. M.; et al. Inhibition of ebola virus by a molecularly engineered banana lectin. PLoS Negl. Trop. Dis. 2019, 13, e0007595.
31. Postnikova, E.; Cong, Y.; DeWald, L. E.; Dyall, J.; Yu, S.; Hart, B. J.; Zhou, H.; Gross, R.; Logue, J.; Cai, Y.; et al. Testing therapeutics in cell-based assays: Factors that influence the apparent potency of drugs. PLoS ONE 2018, 13, e0194880.
32. Nelson, E. A.; Dyall, J.; Hoenen, T.; Barnes, A. B.; Zhou, H.; Liang, J. Y.; Michelotti, J.; Dewey, W. H.; DeWald, L. E.; Bennett, R. S.; et al. The phosphatidylinositol-3-phosphate 5-kinase inhibitor apilimod blocks filoviral entry and infection. PLoS Negl. Trop. Dis. 2017, 11, e0005540.
33. Schornberg, K.; Matsuyama, S.; Kabsch, K.; Delos, S.; Bouton, A.; White, J. Role of endosomal cathepsins in entry mediated by the Ebola virus glycoprotein. J. Virol. 2006, 80, 4174-4178.
34. Shoemaker, C. J.; Schornberg, K. L.; Delos, S. E.; Scully, C.; Pajouhesh, H.; Olinger, G. G.; Johansen, L. M.; White, J. M. Multiple cationic amphiphiles induce a Niemann-Pick C phenotype and inhibit Ebola virus entry and infection. PLoS ONE 2013, 8, e56265.
35. Wu, W. N.; Pritchard, J. F.; Ng, K. T.; Hills, J. F.; Uetz, J. A.; Yorgey, K. A.; McKown, L. A.; O'Neill, P. J. Disposition of bepridil in laboratory animals and man. Xenobiotica 1992, 22, 153-169.
36. DeGregorio, M. W.; Wurz, G. T.; Taras, T. L.; Erkkola, R. U.; Halonen, K. H.; Huupponen, R. K. Pharmacokinetics of (deaminohydroxy)toremifene in humans: A new, selective estrogen-receptor modulator. Eur. J. Clin. Phannacol. 2000, 56, 469-475.
37. Ruderman, E. B. Effects of Acute Aerobic Exercise on the Pharmacokinetics of the Anti-anxiety/Anti-depressant Drug Sertraline. Master's Thesis, University of Toronto, Toronto, Canada, 2013.
38. France, A. Monolix Version 2019R2; Lixoft SAS: Antony, France, 2019.
39. Schiffer, J. T.; Swan, D. A.; Corey, L.; Wald, A. Rapid viral expansion and short drug half-life explain the incomplete effectiveness of current herpes simplex virus 2-directed antiviral agents. Antimicrob. Agents Chemother. 2013, 57, 5820-5829.
40. Schiffer, J. T.; Swan, D. A.; Magaret, A.; Corey, L.; Wald, A.; Ossig, J.; Ruebsamen-Schaeff, H.; Stoelben, S.; Timmler, B.; Zimmermann, H.; et al. Mathematical modeling of herpes simplex virus-2 suppression with pritelivir predicts trial outcomes. Sci. Transl. Med. 2016, 8, 324ra15.
41. Kang, Y.-L.; Chou, Y.-Y.; Rothlauf, P. W.; Liu, Z.; Soh, T. K.; Cureton, D.; Case, J. B.; Chen, R. E.; Diamond, MS.; Whelan, S. P. J.; et al. Inhibition of PlKfyve kinase prevents infection by Zaire ebolavirus and SARS-CoV-2. Proc. Natl. Acad. Sci. USA 2020, 117, 20803-20813.
42. Qiu, S.; Leung, A.; Bo, Y.; Kozak, R. A.; Anand, S. P.; Warkentin, C.; Salambanga, F. D. R.; Cui, J.; Kobinger, G.; Kobasa, D.; et al. Ebola virus requires phosphatidylinositol (3,5) bisphosphate production for efficient viral entry. Virology 2018, 513, 17-28.
43. Wada, Y.; Cardinale, I.; Khatcherian, A.; Chu, J.; Kantor, A. B.; Gottlieb, A. B.; Tatsuta, N.; Jacobson, E.; Barsoum, J.; Krueger, J. G. Apilimod inhibits the production of IL-12 and IL-23 and reduces dendritic cell infiltration in psoriasis. PLoS ONE 2012, 7, e35069.
44. Wada, Y.; Lu, R.; Zhou, D.; Chu, J.; Przewloka, T.; Zhang, S.; Li, L.; Wu, Y.; Qin, J.; Balasubramanyam, V.; et al. Selective abrogation of Th1 response by STA-5326, a potent IL-12/IL-23 inhibitor. Blood 2007, 109, 1156-1164.
45. Rogers, K. J.; Shtanko, O.; Stunz, L. L.; Mallinger, L. N.; Arkee, T.; Schmidt, M. E.; Bohan, D.; Brunton, B.; White, J. M.; Varga, S. M.; et al. Frontline Science: CD40 signaling restricts RNA virus replication in Alt's, leading to rapid innate immune control of acute virus infection. J. Leukoc. Biol. 2020, doi:10.1002/JLB.4HI0420-285RR
46. Morris, P. J.; Moore, C.; Thomas, C. J. Apilimod. IUCrData 2017, 2, 2.
47. DeWald, L. E.; Dyall, J.; Sword, J. M.; Torzewski, L.; Zhou, H.; Postnikova, E.; Kollins, E.; Alexander, I.; Gross, R.; Cong, Y.; et al. The calcium channel blocker bepridil demonstrates efficacy in the murine model of marburg virus disease. J. Infect. Dis. 2018, 218, S588-S591.
48. Honko, A. N.; Johnson, J. C.; Marchand, J. S.; Huzella, L.; Adams, R. D.; Oberlander, N.; Torzewski, L. M.; Bennett, R. S.; Hensley, L. E.; Jahrling, P. B.; et al. High dose sertraline monotherapy fails to protect rhesus macaques from lethal challenge with Ebola virus Makona. Sci. Rep. 2017, 7, 5886.
49. Côté, M.; Misasi, J.; Ren, T.; Bruchez, A.; Lee, K.; Filone, C. M.; Hensley, L.; Li, Q.; Ory, D.; Chandran, K.; et al. Small molecule inhibitors reveal Niemann-Pick C1 is essential for Ebola virus infection. Nature 2011, 477, 344-348.
50. Carette, J. E.; Raaben, M.; Wong, A. C.; Herbert, A. S.; Obernosterer, G.; Mulherkar, N.; Kuehne, A. I.; Kranzusch, P. J.; Griffin, A. M.; Ruthel, G.; et al. Ebola virus entry requires the cholesterol transporter Niemann-Pick C1. Nature 2011, 477, 340-343.
51. Miller, E. H.; Obernosterer, G.; Raaben, M.; Herbert, A. S.; Deffieu, M. S.; Krishnan, A.; Ndungo, E.; Sandesara, R. G.; Carette, J. E.; Kuehne, A. I.; et al. Ebola virus entry requires the host-programmed recognition of an intracellular receptor. EMBO J. 2012, 31, 1947-1960.
52. Spence, J. S.; Krause, T. B.; Mittler, E.; Jangra, R. K.; Chandran, K. Direct visualization of ebola virus fusion triggering in the endocytic pathway. mBio 2016, 7, e01857-15.
53. Simmons, J. A.; D'Souza, R. S.; Ruas, M.; Galione, A.; Casanova, J. E.; White, J. M. Ebolavirus Glycoprotein Directs Fusion through NPC1+Endolysosomes. J. Virol. 2016, 90, 605-610.
54. Mittler, E.; Alkutkar, T.; Jangra, R. K.; Chandran, K. Direct Intracellular Visualization of Ebola Virus-Receptor Interaction by In Situ Proximity Ligation. mBio 2021, 12, 12.
55. Ren, J.; Zhao, Y.; Fry, E. E.; Stuart, D. I. Target Identification and Mode of Action of Four Chemically Divergent Drugs against Ebolavirus Infection. J. Med. Chem. 2018, 61, 724-733.
56. Zhao, Y.; Ren, J.; Harlos, K.; Jones, D. M.; Zeltina, A.; Bowden, T. A.; Padilla-Parra, S.; Fry, E. E.; Stuart, D. I. Toremifene interacts with and destabilizes the Ebola virus glycoprotein. Nature 2016, 535, 169-172.
57. White, J. M.; Whittaker, G. R. Fusion of enveloped viruses in endosomes. Traffic 2016, 17, 593-614.
58. Penny, C. J.; Vassileva, K.; Jha, A.; Yuan, Y.; Chee, X.; Yates, E.; Mazzon, M.; Kilpatrick, B. S.; Muallem, S.; Marsh, M.; et al. Mining of Ebola virus entry inhibitors identifies approved drugs as two-pore channel pore blockers. Biochim. Biophys. Acta Mol. Cell Res. 2019, 1866, 1151-1161.
59. Sakurai, Y.; Kolokoltsov, A. A.; Chen, C.-C.; Tidwell, M. W.; Bauta, W. E.; Klugbauer, N.; Grimm, C.; Wahl-Schott, C.; Biel, M.; Davey, R. A. Two-pore channels control Ebola virus host cell entry and are drug targets for disease treatment. Science 2015, 347, 995-998.
60. Fénéant, L.; Wijs, K. M. S.-D.; Nelson, E. A.; White, J. M. An exploration of conditions proposed to trigger the Ebola virus glycoprotein for fusion. PLoS ONE 2019, 14, e0219312.
61. Hulseberg, C. E.; Feneant, L.; Szymanska, K. M.; White, J. M. Lamp 1 increases the efficiency of lassa virus infection by promoting fusion in less acidic endosomal compartments. mBio 2018, 9, doi:10.1128/mBio.01818-17.
62. Warren, T. K.; Jordan, R.; Lo, M. K.; Ray, A. S.; Mackman, R. L.; Soloveva, V.; Siegel, D.; Perron, M.; Bannister, R.; Hui, H. C.; et al. Therapeutic efficacy of the small molecule GS-5734 against Ebola virus in rhesus monkeys. Nature 2016, 531, 381-385.
63. Bixler, S. L.; Bocan, T. M.; Wells, J.; Wetzel, K. S.; Van Tongeren, S. A.; Dong, L.; Garza, N. L.; Donnelly, G.; Cazares, L. H.; Nuss, J.; et al. Efficacy of favipiravir (T-705) in nonhuman primates infected with Ebola virus or Marburg virus. Antivir. Res. 2018, 151, 97-104.
64. Guedj, J.; Piorkowski, G.; Jacquot, F.; Madelain, V.; Nguyen, T. H. T.; Rodallec, A.; Gunther, S.; Carbonnelle, C.; Mentre, F.; Raoul, H.; et al. Antiviral efficacy of favipiravir against Ebola virus: A translational study in cynomolgus macaques. PLoS Med. 2018, 15, e1002535.
65. DeWald, L. E.; Johnson, J. C.; Gerhardt, D. M.; Torzewski, L. M.; Postnikova, E.; Honko, A. N.; Janosko, K.; Huzella, L.; Dowling, W. E.; Eakin, A. E.; et al. In Vivo Activity of Amodiaquine against Ebola Virus Infection. Sci. Rep. 2019, 9, 20199.
66. Mulangu, S.; Dodd, L. E.; Davey, R. T.; Tshiani Mbaya, O.; Proschan, M.; Mukadi, D.; Lusakibanza Manzo, M.; Nzolo, D.; Tshomba Oloma, A.; Ibanda, A.; et al. A randomized, controlled trial of ebola virus disease therapeutics. N. Engl. J. Med. 2019, 381, 2293-2303.
67. Vernet, M.-A.; Reynard, S.; Fizet, A.; Schaeffer, J.; Pannetier, D.; Guedj, J.; Rives, M.; Georges, N.; Garcia-Bonnet, N.; Sylla, A. I.; et al. Clinical, virological, and biological parameters associated with outcomes of Ebola virus infection in Macenta, Guinea. JCI Insight 2017, 2, e88864.
68. Lanini, S.; Portella, G.; Vairo, F.; Kobinger, G. P.; Pesenti, A.; Langer, M.; Kabia, S.; Brogiato, G.; Amone, J.; Castilletti, C.; et al. INMI-EMERGENCY EBOV Sierra Leone Study Group Blood kinetics of Ebola virus in survivors and nonsurvivors. J. Clin. Investig. 2015, 125, 4692-4698.
69. Geisbert, T. W.; Strong, J. E.; Feldmann, H. Considerations in the use of nonhuman primate models of ebola virus and marburg virus infection. J. Infect. Dis. 2015, 212 Suppl 2, S91-S97.
70. Lane, T. R.; Ekins, S. Toward the target: Tilorone, quinacrine, and pyronaridine bind to ebola virus glycoprotein. ACS Med. Chem. Lett. 2020, 11, 1653-1658.
71. Zhou, N.; Pan, T.; Zhang, J.; Li, Q.; Zhang, X.; Bai, C.; Huang, F.; Peng, T.; Zhang, J.; Liu, C.; et al. Glycopeptide Antibiotics Potently Inhibit Cathepsin L in the Late Endosome/Lysosome and Block the Entry of Ebola Virus, Middle East Respiratory Syndrome Coronavirus (MERS-CoV), and Severe Acute Respiratory Syndrome Coronavirus (SARS-CoV). J. Biol. Chem. 2016, 291, 9218-9232.
72. Hulseberg, C. E.; Feneant, L.; Wij s, K. M. S.-D.; Kessler, N. P.; Nelson, E. A.; Shoemaker, C. J.; Schmaljohn, C. S.; Polyak, S. J.; White, J. M. Arbidol and Other Low-Molecular-Weight Drugs That Inhibit Lassa and Ebola Viruses. J. Virol. 2019, 93, e02185-02118
73. Cooper, L.; Schafer, A.; Li, Y.; Cheng, H.; Medegan Fagla, B.; Shen, Z.; Nowar, R.; Dye, K.; Anantpadma, M.; Davey, R. A.; et al. Screening and Reverse-Engineering of Estrogen Receptor Ligands as Potent Pan-Filovirus Inhibitors. J. Med. Chem. 2020, 63, 11085-11099.
74. Sakurai, Y.; Sakakibara, N.; Toyama, M.; Baba, M.; Davey, R. A. Novel amodiaquine derivatives potently inhibit Ebola virus infection. Antivir. Res. 2018, 160, 175-182.
75. Singleton, C. D.; Humby, M. S.; Yi, H. A.; Rizzo, R. C.; Jacobs, A. Identification of ebola virus inhibitors targeting GP2 using principles of molecular mimicry. J. Virol. 2019, 93, e00676-19.
76. Si, L.; Meng, K.; Tian, Z.; Sun, J.; Li, H.; Zhang, Z.; Soloveva, V.; Li, H.; Fu, G.; Xia, Q.; et al. Triterpenoids manipulate a broad range of virus-host fusion via wrapping the HR2 domain prevalent in viral envelopes. Sci. Adv. 2018, 4, eaau8408.
77. Sheahan, T. P.; Sims, A. C.; Zhou, S.; Graham, R. L.; Pruijssers, A. J.; Agostini, M. L.; Leist, S. R.; Schafer, A.; Dinnon, K. H.; Stevens, L. J.; et al. An orally bioavailable broad-spectrum antiviral inhibits SARS-CoV-2 in human airway epithelial cell cultures and multiple coronaviruses in mice. Sci. Transl. Med. 2020, 12, doi:10.1126/scitranslmed.abb5883.
78. Weston, S.; Baracco, L.; Keller, C.; Matthews, K.; McGrath, M. E.; Logue, J.; Liang, J.; Dyall, J.; Holbrook, M. R.; Hensley, L. E.; et al. The SKI complex is a broad-spectrum, host-directed antiviral drug target for coronaviruses, influenza, and filoviruses. Proc. Natl. Acad. Sci. USA 2020, 117, 30687-30698.
79. Bennett, R. P.; Finch, C. L.; Postnikova, E. N.; Stewart, R. A.; Cai, Y.; Yu, S.; Liang, J.; Dyall, J.; Salter, J. D.; Smith, H. C.; et al. A Novel Ebola Virus VP40 Matrix Protein-Based Screening for Identification of Novel Candidate Medical Countermeasures. Viruses 2020, 13, 52.
80. Lasala, F.; Garcia-Rubia, A.; Requena, C.; Galindo, I.; Cuesta-Geijo, M. A.; Garcia-Dorival, I.; Bueno, P.; Labiod, N.; Luczkowiak, J.; Martinez, A.; et al. Identification Of Potential Inhibitors Of Protein-Protein Interaction Useful To Fight Against Ebola And Other Highly Pathogenic Viruses. Antivir. Res. 2021, 186, 105011.
81. Mallikaarjun, S.; Salazar, D. E.; Bramer, S. L. Pharmacokinetics, tolerability, and safety of aripiprazole following multiple oral dosing in normal healthy volunteers. J. Clin. Pharmacol. 2004, 44, 179-187.
82. Ghobadi, C.; Mirhosseini, N.; Shiran, M. R.; Moghadamnia, A.; Lennard, M. S.; Ledger, W. L.; Rostami-Hodjegan, A. Single-dose pharmacokinetic study of clomiphene citrate isomers in anovular patients with polycystic ovary disease. J. Clin. Pharmacol. 2009, 49, 147-154.
83. Ronfeld, R. A.; Tremaine, L. M.; Wilner, K. D. Pharmacokinetics of sertraline and its N-demethyl metabolite in elderly and young male and female volunteers. Clin Pharmacokinet 1997, 32 Suppl 1, 22-30.
84. Anttila, M.; Valavaara, R.; Kivinen, S.; Maenpaa, J. Pharmacokinetics of toremifene. J Steroid Biochem 1990, 36, 249-252.
85. Madrid, P. B.; Panchal, R. G.; Warren, T. K.; Shurtleff, A. C.; Endsley, A. N.; Green, C. E.; Kolokoltsov, A.; Davey, R.; Manger, I. D.; Gilfillan, L.; Bavari, S.; Tanga, M. J. Evaluation of ebola virus inhibitors for drug repurposing. ACS Infect. Dis. 2015, 1, 317-326.
86. Bobrowski, T. et al. Synergistic and antagonistic drug combinations against SARS-CoV-2. Molecular Therapy 2021, 29, 873-885.
87. Chaudhuri, S., Symons, J. A. & Deval, J. Innovation and trends in the development and approval of antiviral medicines: 1987-2017 and beyond. Antiviral research 2018, 155, 76-88.
88. Andersen, P. I. et al. Discovery and development of safe-in-man broad-spectrum antiviral agents. International Journal of Infectious Diseases 2020, 93, 268-276.
89. Ianevski, A., Andersen, P. I., Merits, A., Bjørås, M. & Kainov, D. Expanding the activity spectrum of antiviral agents. Drug discovery today 2019, 24, 1224-1228.
90. Pizzorno, A., Padey, B., Terrier, O. & Rosa-Calatrava, M. Drug repurposing approaches for the treatment of influenza viral infection: reviving old drugs to fight against a long-lived enemy. Frontiers in immunology 2019, 10, 531.
91. Foucquier, J. & Guedj, M. Analysis of drug combinations: current methodological landscape. Pharmacology research & perspectives 2015, 3, e00149.
92. Sun, W., Sanderson, P. E. & Zheng, W. Drug combination therapy increases successful drug repositioning. Drug discovery today 2016, 21, 1189-1195.
93. Zheng, W., Sun, W. & Simeonov, A. Drug repurposing screens and synergistic drug-combinations for infectious diseases. British journal of pharmacology 2018, 175, 181-191.
94. Bösl, K. et al. Common nodes of virus-host interaction revealed through an integrated network analysis. Frontiers in immunology 2019, 10, 2186.
95. dos Santos Nascimento, I. J., de Aquino, T. M. & da Silva-Júnior, E. F. Drug repurposing: A strategy for discovering inhibitors against emerging viral infections. Current Medicinal Chemistry 2021, 28, 2887-2942.
96. Pires de Mello, C. P. et al. Zika virus replication is substantially inhibited by novel favipiravir and interferon alpha combination regimens. Antimicrobial agents and chemotherapy 2018, 62, e01983-01917.
97. Snyder, B., Goebel, S., Koide, F., Ptak, R. & Kalkeri, R. Synergistic antiviral activity of Sofosbuvir and type-I interferons (α and β) against Zika virus. Journal of medical virology 2018, 90, 8-12.
98. Herring, S. et al. Inhibition of arenaviruses by combinations of orally available approved drugs. Antimicrobial agents and chemotherapy 2021, 65, e01146-01120.
99. Pizzorno, A. et al. In vitro evaluation of antiviral activity of single and combined repurposable drugs against SARS-CoV-2. Antiviral research 2020, 181, 104878.
100. Ianevski, A. et al. Identification and tracking of antiviral drug combinations. Viruses 2020, 12, 1178.
101. Ianevski, A., Giri, A. K. & Aittokallio, T. SynergyFinder 2.0: visual analytics of multi-drug combination synergies. Nucleic acids research 2020, 48, W488-W493.
102. Ianevski, A., He, L., Aittokallio, T. & Tang, J. SynergyFinder: a web application for analyzing drug combination dose-response matrix data. Bioinformatics 2017, 33, 2413-2415.
103. Prichard, M. N. & Shipman Jr, C. A three-dimensional model to analyze drug-drug interactions. Antiviral research 1990, 14, 181-205.
104. Prichard, M., Aseltine, K. & Shipman Jr, C. MacSynergy™ II. University of Michigan. Ann Arbor (1992).
105. Berenbaum, M. C. What is synergy? Pharmacological reviews 1989, 41, 93-141.
106. Loewe, S. The problem of synergism and antagonism of combined drugs. Arzneimittelforschung 1953, 3, 285-290.
107. Bliss, C. I. The toxicity of poisons applied jointly 1. Annals of applied biology 1939, 26, 585-615.
108. Yadav, B., Wennerberg, K., Aittokallio, T. & Tang, J. Searching for drug synergy in complex dose-response landscapes using an interaction potency model. Computational and structural biotechnology journal 2015, 13, 504-513.
109. Wicha, S. G., Chen, C., Clewe, O. & Simonsson, U.S.A general pharmacodynamic interaction model identifies perpetrators and victims in drug interactions. Nature communications 2017, 8, 1-11.
110. Derendorf, H. & Meibohm, B. Modeling of pharmacokinetic/pharmacodynamic (PK/PD) relationships: concepts and perspectives. Pharmaceutical research 1999, 16,176-185.
111. Rajman, I. PK/PD modelling and simulations: utility in drug development. Drug discovery today 2008, 13, 341-346.
112. Park, K. A review of modeling approaches to predict drug response in clinical oncology. Yonsei medical journal 2017, 58, 1-8.
113. Maharao, N., Antontsev, V., Wright, M. & Varshney, J. Entering the era of computationally driven drug development. Drug metabolism reviews 2020, 52, 283-298.
114. Wicha, S. G., Kees, M. G., Kuss, J. & Kloft, C. Pharmacodynamic and response surface analysis of linezolid or vancomycin combined with meropenem against Staphylococcus aureus. Pharmaceutical research 2015, 32, 2410-2418.
115. Box, G. E. & Draper, N. R. Empirical model-building and response surfaces. (John Wiley & Sons, 1987).
116. Liu, Q., Yin, X., Languino, L. R. & Altieri, D. C. Evaluation of drug combination effect using a bliss independence dose-response surface model. Statistics in biopharmaceutical research 2018, 10, 112-122.
117. Pecheur, E.-I. et al. The synthetic antiviral drug arbidol inhibits globally prevalent pathogenic viruses. Journal of virology 2016, 90, 3086-3092.
118. Lo, M. K. et al. GS-5734 and its parent nucleoside analog inhibit Filo-, Pneumo-, and Paramyxoviruses. Scientific reports 2017, 7, 1-7.
119. Sheahan, T. P. et al. Broad-spectrum antiviral GS-5734 inhibits both epidemic and zoonotic coronaviruses. Science translational medicine 2017, 9, eaa13653.
120. Hakamaki, T., Apoil, E., Arstila, M., Timmer, C. & Lehtonen, A. Bepridil in the elderly. A pharmacokinetic and clinical monitoring study. Current therapeutic research 1988, 44, 752-758.
121. Sun, Y. et at Pharmacokinetics of single and multiple oral doses of arbidol in healthy Chinese volunteers. International journal of clinical pharmacology and therapeutics 2013, 51, 423-432.
122. Haffizulla, J. et al. Effect of nitazoxanide in adults and adolescents with acute uncomplicated influenza: a double-blind, randomised, placebo-controlled, phase 2b/3 trial. The Lancet Infectious diseases 2014, 14, 609-618.
123. Humeniuk, R. et al. Safety, tolerability, and pharmacokinetics of remdesivir, an antiviral for treatment of COVID-19, in healthy subjects. Clinical and translational science 2020, 13, 896-906.
124. Goyal A, Cardozo-Ojeda E F, Schiffer J T. Potency and timing of antiviral therapy as determinants of duration of SARS-CoV-2 shedding and intensity of inflammatory response. Sci. Adv. 2020, 6, eabc7112.
125. Boffito M, Back D J, Flexner C, Sjo P, Blaschke T F, Horby P W, Cattaneo D, Acosta E P, Anderson P, Owen A. Toward Consensus on Correct Interpretation of Protein Binding in Plasma and Other Biological Matrices for COVID-19 Therapeutic Development. Clin. Pharmacol. Ther. 2021, 110, 64-68.
126. Smith D A, Di L, Kerns E H. The effect of plasma protein binding on in vivo efficacy: misconceptions in drug discovery. Nat. Rev. Drug Discov. 2010, 9, 929-939.
127. Wölfel R, Corman V M, Guggemos W, Seilmaier M, Zange S, Müller MA, Niemeyer D, Jones T C, Vollmar P, Rothe C, Hoelscher M, Bleicker T, Briinink S, Schneider J, Ehmann R, Zwirglmaier K, Drosten C, Wendtner C. Virological assessment of hospitalized patients with COVID-2019. Nature 2020, 581, 465-469.
128. Corey L, Beyrer C, Cohen M S, Michael N L, Bedford T, Rolland M. SARS-CoV-2 Variants in Patients with Immunosuppression. N. Engl. J. Med. 2021, 385, 562-566.
129. Avanzato V A, Matson M J, Seifert S N, Pryce R, Williamson B N, Anzick S L, Barbian K, Judson S D, Fischer E R, Martens C, Bowden T A, de Wit E, Riedo F X, Munster V J. Case Study: Prolonged Infectious SARS-CoV-2 Shedding from an Asymptomatic Immunocompromised Individual with Cancer. Cell 2020, 183, 1901-1912.e9.
130. Choi B et al. Persistence and Evolution of SARS-CoV-2 in an Immunocompromised Host. N. Engl. J. Med. 2020, 383, 2291-2293.
131. Truong T T. et al. Persistent SARS-CoV-2 infection and increasing viral variants in children and young adults with impaired humoral immunity. medRxiv 2021, preprint, PMID:33688673.
132. Kemp S A et al. SARS-CoV-2 evolution during treatment of chronic infection. Nature 2021, 592, 277-282.
133. Lee J M, Eguia R, Zost S J, Choudhary S, Wilson P C, Bedford T, Stevens-Ayers T, Boeckh M, Hurt A C, Lakdawala S S, Hensley S E, Bloom J D. Mapping person-to-person variation in viral mutations that escape polyclonal serum targeting influenza hemagglutinin. Elife 2019, 8, e49324.
134. Meganck R M, Baric R S. Developing therapeutic approaches for twenty-first-century emerging infectious viral diseases. Nat. Med. 2021, 27,401-410.
135. White J M, Delos S E, Brecher M, Schomberg K. Structures and mechanisms of viral membrane fusion proteins: multiple variations on a common theme. Crit. Rev. Biochem. Mol. Biol. 2008, 43, 189-219.
136. Toelzer C, Gupta K, Yadav S K N, Borucu U, Davidson A D, Kavanagh Williamson M, Shoemark D K, Garzoni F, Staufer O, Milligan R, Capin J, Mulholland A J, Spatz J, Fitzgerald D, Berger I, Schaffitzel C. Free fatty acid binding pocket in the locked structure of SARS-CoV-2 spike protein. Science 2020, 370, 725-730.
137. Hoffman L R, Kuntz I D, White J M. Structure-based identification of an inducer of the low-pH conformational change in the influenza virus hemagglutinin: irreversible inhibition of infectivity. J. Virol. 1997, 71, 8808-8820.
138. Bodian D L, Yamasaki R B, Buswell R L, Stearns J F, White J M, Kuntz I D. Inhibition of the fusion-inducing conformational change of influenza hemagglutinin by benzoquinones and hydroquinones. Biochemistry 1993, 32, 2967-2978.
139. Narkhede Y B, Gonzalez K J, Strauch E-M. Targeting Viral Surface Proteins through Structure-Based Design. Viruses 2021, 13, 1320.
140. Xia S et al. Structural and functional basis for pan-CoV fusion inhibitors against SARS-CoV-2 and its variants with preclinical evaluation. Signal Transduct. Target Ther. 2021, 6, 288.
141. Case J B et al. Neutralizing Antibody and Soluble ACE2 Inhibition of a Replication-Competent VSV-SARS-CoV-2 and a Clinical Isolate of SARS-CoV-2. Cell Host Microbe 2020, 28, 475-485.e5.
142. Luo D, Vasudevan S G, Lescar J. The flavivirus NS2B-NS3 protease-helicase as a target for antiviral drug development. Antiviral Res. 2015, 118, 148-158.
143. Das P K, Puusepp L, Varghese F S, Utt A, Ahola T, Kananovich D G, Lopp M, Merits A, Karelson M. Design and validation of novel chikungunya virus protease inhibitors. Antimicrob. Agents Chemother. 2016, 60, 7382-7395.
144. Mazzon M, Ortega-Prieto A M, Imrie D, Luft C, Hess L, Czieso S, Grove J, Skelton J K, Farleigh L, Bugert J J, Wright E, Temperton N, Angell R, Oxenford S, Jacobs M, Ketteler R, Domer M, Marsh M. Identification of Broad-Spectrum Antiviral Compounds by Targeting Viral Entry. Viruses 2019, 11, 176.
145. Yamauchi Y, Helenius A. Virus entry at a glance. J Cell Sci. 2013, 126, 1289-1295.
146. Drews K, Calgi M P, Harrison W C, Drews C M, Costa-Pinheiro P, Shaw J J P, Jobe K A, Nelson E A, Han J D, Fox T, White J M, Kester M. Glucosylceramidase maintains influenza virus infection by regulating endocytosis. J. Virol. 2019, 93, e00017-19.
147. Zaitseva E, Yang S-T, Melikov K, Pourmal S, Chernomordik L V. Dengue virus ensures its fusion in late endosomes using compartment-specific lipids. PLoS Pathog. 2010, 6, e1001131.
148. Pager C T, Craft W W, Patch J, Dutch R E. A mature and fusogenic form of the Nipah virus fusion protein requires proteolytic processing by cathepsin L. Virology 2006, 346, 251-257.
149. Hoffmann M et al. SARS-CoV-2 Cell Entry Depends on ACE2 and TMPRSS2 and Is Blocked by a Clinically Proven Protease Inhibitor. Cell 2020, 181, 271-280.e8.
150. Whittaker G R, Daniel S, Millet J K. Coronavirus entry: how we arrived at SARS-CoV-2. Curr. Opin. Virol. 2021, 47, 113-120.
151. Koch J, Uckeley Z M, Doldan P, Stanifer M, Boulant S, Lozach P-Y. TMPRSS2 expression dictates the entry route used by SARS-CoV-2 to infect host cells. EMBO J. 2021, e107821.
152. Kadam R U, Wilson I A. Structural basis of influenza virus fusion inhibition by the antiviral drug Arbidol. Proc. Natl. Acad. Sci. USA 2017, 114, 206-214.
153. Schafer A, Xiong R, Cooper L, Nowar R, Lee H, Li Y, Ramirez B E, Peet N P, Caffrey M, Thatcher G R J, Saphire E O, Cheng H, Rong L. Evidence for distinct mechanisms of small molecule inhibitors of filovirus entry. PLoS Pathog. 2021, 17, e1009312.
154. Peacock T P et al. The furin cleavage site in the SARS-CoV-2 spike protein is required for transmission in ferrets. Nat. Microbiol. 2021, 6, 899-909.
155. Hoffmann M, Kleine-Weber H, Pohlmann S. A Multibasic Cleavage Site in the Spike Protein of SARS-CoV-2 Is Essential for Infection of Human Lung Cells. Mol. Cell 2020, 78, 779-784.e5.
156. Sarute N, Cheng H, Yan Z, Salas-Briceno K, Richner J, Rong L, Ross S R. Signal-regulatory protein alpha is an anti-viral entry factor targeting viruses using endocytic pathways. PLoS Pathog. 2021, 17, e1009662.
157. Bouhaddou M et al. The Global Phosphorylation Landscape of SARS-CoV-2 Infection. Cell, 2020, 182, 685-712.e19.
158. Drayman N et al. Masitinib is a broad coronavirus 3CL inhibitor that blocks replication of SARS-CoV-2. Science 2021, 373, 931-936.
159. Saul S et al. Discovery of pan-ErbB inhibitors protecting from SARS-CoV-2 replication, inflammation, and lung injury by a drug repurposing screen. BioRxiv, 2021, preprint, 444128.
160. Meyer B et al. Characterising proteolysis during SARS-CoV-2 infection identifies viral cleavage sites and cellular targets with therapeutic potential. Nat. Commun. 2021, 12, 5553.
161. Williams C G, Jureka A S, Silvas J A, Nicolini A M, Chvatal S A, Carlson-Stevermer J, Oki J, Holden K, Basler CFInhibitors of VPS34 and fatty-acid metabolism suppress SARS-CoV-2 replication. Cell Rep. 2021, 109479.
162. Kashyap T, Murray J, Walker C J, Chang H, Tamir S, Hou B, Shacham S, Kauffman M G, Tripp R A, Landesman Y. Selinexor, a novel selective inhibitor of nuclear export, reduces SARS-CoV-2 infection and protects the respiratory system in vivo. Antiviral Res. 2021, 192, 105115.
163. Jorquera P A, Mathew C, Pickens J, Williams C, Luczo J M, Tamir S, Ghildyal R, Tripp R A. Verdinexor (KPT-335), a Selective Inhibitor of Nuclear Export, Reduces Respiratory Syncytial Virus Replication In Vitro. J. Virot 2019, 93, e01684-18.
164. Han Z, Lu J, Liu Y, Davis B, Lee M S, Olson M A, Ruthel G, Freedman B D, Schnell M J, Wrobel J E, Reitz A B, Harty R N. Small-molecule probes targeting the viral PPxY-host Nedd4 interface block egress of a broad range of RNA viruses. J. Virol. 2014, 88, 7294-7306.
165. Wan J J, Brown R S, Kielian M. Berberine chloride is an alphavirus inhibitor that targets nucleocapsid assembly. MBio. 2020, 11. DOI: 10.1128/mBio.01382-20.
166. Pujadas E, Chaudhry F, McBride R, Richter F, Zhao S, Wajnberg A, Nadkarni G, Glicksberg B S, Houldsworth J, Cordon-Cardo C. SARS-CoV-2 viral load predicts COVID-19 mortality. Lancet Respir Med. 2020, 8, e70.
167. U.S. Pat. No. 8,475,804.

It will be understood that various details of the presently disclosed subject matter may be changed without departing from the scope of the presently disclosed subject matter. Furthermore, the foregoing description is for the purpose of illustration only, and not for the purpose of limitation.

1. A method of modeling an in vivo efficacy of a drug combination in the treatment of a viral infection of a target virus in a target host subject, the method comprising:
(a) acquiring or preparing a first data set, wherein the first data set comprises pharmacokinetic (PK) data for a first drug and a second drug;
(b) acquiring or preparing a second data set, wherein the second data set comprises pharmacodynamic (PD) data for the first drug, the second drug, and a combination of the first drug and the second drug;
(c) acquiring or preparing a third data set, wherein the third data set comprises viral load dynamics data for the target virus in a relevant host subject;
(d) combining the first, second and third data sets using a plurality of projected in vivo drug potencies against the target virus for each of the first drug, the second drug, and the combination of the first drug and the second drug, thereby predicting a plurality of different viral load trajectories in the target host subject; and
(e) analyzing the plurality of different viral load trajectories predicted in step (d) to predict in vivo efficacy of the combination of the first drug and the second drug at one or more of the plurality of projected in vivo drug potencies.

2. The method of claim 1, wherein the method further comprises administering the combination of the first drug and the second drug to the target subject or a surrogate animal subject thereof, wherein the administering comprises administering the combination of the first drug and the second drug at dose levels and/or dosing intervals predicted to be effective in vivo by the analyzing of step (e).

3. The method of claim 1, wherein step (e) comprises predicting the maximum time interval between initial exposure of a target subject to the target virus and an initial dose administration of the combination of the first drug and the second drug to the subject that provides a predetermined level of control of in vivo viral load; and the method further comprises administering an initial dose of the combination of the first drug and the second drug to the target subject or a surrogate animal subject thereof within the predicted maximum time interval.

4. The method of claim 1, wherein the target virus is a virus selected from the group consisting of arenaviruses, bunyaviruses, calciviruses, filoviruses, flaviviruses, orthomyxoviruses, picornaviruses, rhabdoviruses, togaviruses, influenza viruses and coronaviruses.

5. The method of claim 1, wherein step (b) comprises performing two-way concentration-dependent analysis of drug-drug interactions between the first drug and the second drug.

6. The method of claim 1, wherein the drug combination comprises a third drug, and wherein the first data set comprises PK data for the third drug; wherein the second data set comprises PD data for the third drug, a combination of the first drug and the third drug, a combination of the second drug and the third drug, and a combination of the first drug, the second drug and the third drug; wherein step (d) comprises combining the first, second and third data sets using a plurality of projected in vivo drug potencies against the target virus for each of the first drug, the second drug, third drug, the combination of the first drug and the second drug, the combination of the first drug and the third drug, the combination of the second drug and the third drug, and the combination of the first drug, the second drug and the third drug; and wherein step (e) comprises analyzing the plurality of different viral load trajectories predicted in step (d) to predict in vivo efficacy of the combination of the first drug and the second drug, the first drug and the third drug, the second drug and the third drug, and the first drug, the second drug and the third drug at one or more of the plurality of projected in vivo drug potencies.

7. A method of treating or preventing a filovirus infection in an animal subject in need thereof, the method comprising orally administering to said animal subject one of
(a) a combination of bepridil or a pharmaceutically acceptable salt thereof and sertraline or a pharmaceutically acceptable salt thereof such that the combination provides a dose of 300 milligrams per day (mg/D) of bepridil and a dose of 200 mg/D of sertraline; or
(b) a combination of sertraline or a pharmaceutically acceptable salt thereof and toremifene or a pharmaceutically acceptable salt thereof such that the combination provides a dose of 200 mg/D of sertraline and a dose of 150 mg/D of toremifene.

8. The method of claim 7, wherein the filovirus infection is an infection of an Ebolavirus species.

9. The method of claim 8, wherein the Ebolavirus species is Zaire ebolavirus.

10. The method of claim 7, wherein the filovirus infection is an infection of a Marburgvirus species.

11. The method of claim 10, wherein the Marburgvirus species is Marburg marburgvirus.

12. The method of claim 7, wherein the animal subject is a human.

13. The method of claim 7, wherein the orally administering is first performed within about 1 day of exposure or suspected exposure of the subject to a filovirus that causes the filovirus infection.

14. The method of claim 7, wherein the administering is performed daily for ten days.

15. The method of claim 7, wherein the method further comprises administering one or more additional therapeutic agents to the subject.

16. The method of claim 15, wherein the one or more additional therapeutic agents comprise an antiviral agent.

17. Composition for use in a method of treating or preventing a filovirus infection in an animal subject in need thereof, wherein the composition comprises:
(a) a combination of bepridil or a pharmaceutically acceptable salt thereof and sertraline or a pharmaceutically acceptable salt thereof such that the combination provides a dose of 300 milligrams per day (mg/D) of bepridil and a dose of 200 mg/D of sertraline; or
(b) a combination of sertraline or a pharmaceutically acceptable salt thereof and toremifene or a pharmaceutically acceptable salt thereof such that the combination provides a dose of 200 mg/D of sertraline and a dose of 150 mg/D of toremifene, and wherein the composition is administered orally to said animal subject.

18. Composition for use according to claim 17, wherein the composition is provided in a dosage form wherein a combination preparation comprising a mixture of:
(a) bepridil or a pharmaceutically acceptable salt thereof and sertraline or a pharmaceutically acceptable salt thereof, or
(b) sertraline or a pharmaceutically acceptable salt thereof and toremifene or a pharmaceutically acceptable salt thereof,
is provided in a single dosage form.

19. Composition for use according to claim 17, wherein the composition is provided in a dosage form wherein two separate individual preparations of:
(a) bepridil or a pharmaceutically acceptable salt thereof and sertraline or a pharmaceutically acceptable salt thereof, or
(b) sertraline or a pharmaceutically acceptable salt thereof and toremifene or a pharmaceutically acceptable salt thereof,
are provided in two separate single dosage forms.
